(19) United States
(12) Patent Application Publication
(10) Pub. No.: US 2026/0009049 A1
(43) Pub. Date: Jan. 8, 2026

(54) COMPOSITIONS FOR TREATING XLMTM

(71) Applicant: KATE THERAPEUTICS, INC., San Diego, CA (US)

(72) Inventors: Mark Fielden, San Diego, CA (US); Mohammadsharif Tabebordbar, San Diego, CA (US); Saurav Seshadri, San Diego, CA (US)

(21) Appl. No.: 18/868,544

(22) PCT Filed: May 11, 2023
(86) PCT No.: PCT/US2023/066865

§ 371 Date: Nov. 22, 2024

Related U.S. Application Data
(60) Provisional application No. 63/345,144, filed on May 24, 2022.

(51) Int. Cl. C12N 15/86; A61K 35/76; A61K 38/46; A61K 48/00; A61P 21/00; C07K 14/005; C12N 9/16

(52) U.S. Cl. CPC C12N 15/86; A61K 35/76; A61K 38/465; A61K 48/0041; A61K 48/0058; A61P 21/00; C07K 14/005; C12N 9/16; C12Y 301/03064

(57) ABSTRACT

The present invention provides a novel muscle-tropic viral vector that achieves MTM1 expression in skeletal muscle. Advantageously, by increasing expression of MTM1 in skeletal muscle, the vector of the present allows for administration of a dose of the composition comprising the vector that substantially reduces the exposure of non-target tissues to the composition.

### FIELD OF DISCLOSURE

This disclosure relates methods for treating X-linked myotubular myopathy (XLMTM).

### SEQUENCE LISTING

The instant application contains a Sequence Listing in XML format, which is hereby incorporated by reference in its entirety. Said XML copy, created on Sep. 29, 2025, is named “51037-095001_Sequence Listing_9_29_25” and is 2,027,680 bytes in size.

### BACKGROUND

X-linked myotubular myopathy (XLMTM) is a rare, grievous congenital neuromuscular disease, occurring in approximately 1 in 50,000 live male births. XLMTM results from pathogenic variants in the myotubularin (MTM1) gene, which encodes the protein myotubularin, a ubiquitously expressed lipid phosphatase that regulates intracellular membrane trafficking and vesicular transport, and whose function is required for the normal development, maturation, and maintenance of skeletal muscle. Reductions in functional myotubularin are associated with the profound skeletal muscle weakness responsible for the most prominent clinical manifestations of this disease.

Nearly all boys with XLMTM present at birth with abnormal Apgar scores, severe hypotonia and weakness, and respiratory distress; approximately 90% require mechanical respiratory support. Affected boys have dramatically shortened lifespan, dying at a median age of approximately 18 months, usually due to respiratory failure and its complications.

The quality of life for the vast majority of affected boys is also poor, as highlighted in a recently published chart review. Patients spend approximately one-third to one-half of their first year of life in the hospital, and rehospitalization for pneumonia and other respiratory indications is common. Even with aggressive supportive therapy to prolong life, boys with XLMTM reach motor milestones late if ever, and rarely achieve independent locomotion. Most require lifelong mechanical respiratory support; approximately half require mechanical ventilation 24 hours per day. Surgical procedures are also common; tracheostomy is often required for patients with long-term ventilator dependence, and nearly all patients require gastrostomy due to weakness impairing feeding ability.

Advances in diagnostic techniques, and particularly genetic testing, have accelerated the time to diagnosis of XLMTM, now typically made within the first 3 to 4 months of life. Therapeutic development efforts are ongoing, but there are currently no approved medicines for the treatment of XLMTM. Supportive care, as described above, can prolong survival but does not alter the course of the disease.

Therapeutic strategies under investigation include AT132 (resamirigene bilparvovec), an intravenously delivered gene therapy consisting of an AAV8 capsid that delivers human MTM1 with a desmin promoter. To date, selected data has been publicly presented on 23 participants who have received AT132 in a Phase 1/2 clinical trial (ASPIRO, NCT03199469). This approach has shown substantial improvements in clinical outcomes, with many participants experiencing improvements in motor function as assessed by CHOP-INTEND, and major reductions in ventilator support requirements, with some participants gaining complete freedom from mechanical ventilation.

Unfortunately, AT132 has also been associated with serious safety findings, notably severe intrahepatic cholestasis leading to liver failure and death in four participants. Liver toxicity has been reported with other gene therapies, typically with prominent elevations in transaminases and resolving with supportive care, sometimes combined with steroids. In contrast, hepatotoxicity in the AT132 program has been characterized by cholestasis, which in some cases has been severe, progressive, and reportedly not responsive to immunosuppression nor prevented by addition of prophylactic ursodeoxycholic acid.

As a consequence, there remain no approved medicines for the treatment of XLMTM, leaving the population of those suffering form the condition with a great unmet medical need.

### SUMMARY

The present invention provides a novel muscle-tropic viral vector that achieves MTM1 expression in skeletal muscle. Advantageously, by increasing expression of MTM1 in skeletal muscle, the vector of the present invention allows for administration of a dose of the composition comprising the vector that substantially reduces the exposure of non-target tissues to the composition. The more effective dosing enables methods of treating XLMTM with clinical efficacy (improvements in muscle strength and reduced need for mechanical ventilation) and with improved safety, in particular, reduced risk of hepatoxicity in this population with great unmet medical need.

Methods of the invention provide an adeno-associated virus (AAV) vector comprising a capsid protein with at least one modification that results in preferential targeting of the AAV vector to muscle tissue. The vector further comprises a nucleic acid encoding a full-length MTM1 protein.

The capsid protein may further comprise at least one modification that results in reduced liver-tropism of the AAV vector.

The AAV may be any known AAV, for example AAV9. The capsid protein may comprise at least one modification that is an insertion between any two contiguous amino acids between amino acids 262-269, 327-332, 382-386, 452-460, 488-505, 527-539, 545-558, 581-593, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh. 10 capsid polypeptide. For example, the capsid protein may comprise at least one modification that is a replacement of amino acids 586-588 and an insertion between amino acids 588 and 589 in an AAV9 capsid polypeptide or in an analogous position in an AAV1, AAV2, AAV3, AAV4, AAV5, AAV6, AAV7, AAV8, AAV rh.74, AAV rh. 10 capsid polypeptide. The capsid protein may comprise at least one modification that is a replacement of amino acids 586-588 and an insertion between amino acids 588 and 589 in an AAV9 capsid polypeptide and wherein the insertion is selected from the sequences in Table 1-4, provided in detail below.

The vector may comprise a vp1, vp2, and vp3 capsid protein. The amino acid sequence of the vp1 capsid protein may be selected from the sequences in Table 5, the amino acid sequence vp2 capsid protein may be selected from the sequences in Table 6, and/or the amino acid sequence vp3 capsid protein is selected from the sequences in Table 7, each of which is provided in detail below.

The nucleic acid encoding the full-length MTM1 protein may be operably linked to a muscle specific promoter. The muscle specific promoter may be any known muscle specific promoter. For example, the muscle specific promoter is an MHCK7 promoter.

The nucleic acid encoding the full-length MTM1 protein may comprise an alternatively-spliced exon cassette downstream of the muscle specific promoter. The alternatively-sliced exon cassette may comprise an ATG start codon at the 3′ end of the cassette. The alternatively-spliced exon cassette may comprise a skeletal muscle-specific exon. Advantageously, the alternatively spliced exon cassette may promote skeletal muscle expression of the nucleic acid. As a consequence, both the AAV capsid and the nucleic acid encoding MTM1 delivered by the capsid both result in increased skeletal muscle expression.

Aspects of the present invention provide methods of treating X-linked myotubular myopathy (XLMTM). The method comprises administering to a subject afflicted with XLMTM a composition comprising an adeno-associated virus (AAV) vector comprising a capsid protein comprising at least one modification that results in in preferential targeting of the AAV vector to muscle tissue and a nucleic acid encoding a full-length MTM1 protein.

The capsid protein may further comprise at least one modification that results in reduced liver-tropism of the AAV vector.

The AAV may be any known AAV, for example AAV9. The capsid protein may comprise at least one modification that is an insertion between any two contiguous amino acids between amino acids 262-269, 327-332, 382-386, 452-460, 488-505, 527-539, 545-558, 581-593, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide. For example, the capsid protein may comprise at least one modification that is a replacement of amino acids 586-88 and an insertion between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide. The capsid protein may comprise at least one modification that is a replacement of amino acids 586-88 and an insertion between amino acids 588 and 589 in an AAV9 capsid polypeptide and wherein the insertion is selected from the sequences in Table 1-4, provided in detail below.

The vector may comprise a vp1, vp2, and vp3 capsid protein. The amino acid sequence of the vp1 capsid protein may be selected from the sequences in Table 5, the amino acid sequence vp2 capsid protein may be selected from the sequences in Table 6, and/or the amino acid sequence vp3 capsid protein is selected from the sequences in Table 7, each of which is provided in detail below.

The nucleic acid encoding the full-length MTM1 protein may be operably linked to a muscle specific promoter. The muscle specific promoter may be any known muscle specific promoter. For example, the muscle specific promoter is an MHCK7 promoter.

The nucleic acid encoding the full-length MTM1 protein may comprise an alternatively-spliced exon cassette downstream of the muscle specific promoter. The alternatively-sliced exon cassette may comprise an ATG start codon at the 3′ end of the cassette. The alternatively-spliced exon cassette may comprise a skeletal muscle-specific exon. Advantageously, the alternatively spliced exon cassette may promote skeletal muscle expression of the nucleic acid. As a consequence, both the AAV capsid and the nucleic acid encoding MTM1 delivered by the capsid both result in increased skeletal muscle expression.

TABLE 1
MyoAAV (eMyoAAV) Capsid Variants

| | | SEQ | | SEQ |
|---|---|---|---|---|
| | | ID | | ID |
| RANK | N-MER MOTIF | NO.: | ENCODING SEQUENCE | NO.: |
| 1 | AEGRGDQYTR | 177 | GCTGAGGGTAGAGGAGACCAGTATACTCGT | 181 |
| 2 | GPGRGDQTTL | 178 | GGGCCGGGTAGAGGAGACCAGACTACGTTG | 182 |
| 3 | AEGRGDQYTR | 177 | GCGGAAGGCAGAGGAGACCAATACACAAGG | 183 |
| 4 | ATGRGDLGQA | 179 | GCGACTGGTAGAGGAGACCTGGGTCAGGCT | 184 |
| 5 | AVARGDQGLI | 180 | GCGGTGGCGAGAGGAGACCAGGGTCTTATT | 185 |
| 6 | NISRGDQGYQ | 6 | AACATCTCCAGAGGAGACCAAGGTTACCAA | 186 |
| 7 | APARGDQGSQ | 7 | GCGCCGGCTAGAGGAGACCAGGGGAGTCAG | 187 |
| 8 | AVSRGDRMEF | 8 | GCCGTTAGCAGAGGAGACCGGATGGAATTC | 188 |
| 9 | QMGRGDMGIK | 10 | CAGATGGGTAGAGGAGACATGGGGATTAAG | 189 |
| 10 | EYRRGDKADI | 11 | GAGTATCGGAGAGGAGACAAGGCGGATATT | 190 |
| 11 | ESRRGDKEPL | 12 | GAGAGTCGGAGAGGAGACAAGGAGCCGCTG | 191 |
| 12 | AHMRGDLGGT | 13 | GCACACATGAGAGGAGACCTAGGCGGCACG | 192 |
| 13 | VWQRGDKMDM | 14 | GTGTGGCAAAGAGGAGACAAAATGGACATG | 193 |
| 14 | SIGRGDTGHM | 15 | TCTATTGGGAGAGGAGACACGGGTCATATG | 194 |
| 15 | NIARGDAGQY | 16 | AATATTGCTAGAGGAGACGCTGGTCAGTAT | 195 |
| 16 | AVARGDQGLI | 180 | GCAGTAGCAAGAGGAGACCAAGGCTTAATC | 196 |
| 17 | SVSRGDQGLH | 17 | TCGGTCTCGAGAGGAGACCAAGGATTGCAC | 197 |
| 18 | EYRRGDKADI | 11 | GAATACAGGAGAGGAGACAAAGCAGACATC | 198 |
| 19 | AHMRGDLGGT | 13 | GCGCATATGAGAGGAGACTTGGGGGGGACT | 199 |
| 20 | QIGRGDITHG | 18 | CAGATTGGTAGAGGAGACATTACTCATGGG | 200 |
| 21 | EVRRGDLHGT | 19 | GAAGTCAGAAGAGGAGACTTGCACGGGACA | 201 |
| 22 | SVSRGDVHTM | 20 | AGTGTCTCAAGAGGAGACGTGCACACGATG | 202 |
| 23 | MVTRGDLGTR | 21 | ATGGTGACTAGAGGAGACCTTGGTACGCGG | 203 |
| 24 | NGGRGDTTHF | 22 | AACGGCGGGAGAGGAGACACGACGCACTTC | 204 |
| 25 | TMGRGDMNSL | 23 | ACGATGGGGAGAGGAGACATGAATTCGCTT | 205 |
| 26 | AISRGDQGLS | 24 | GCGATTAGTAGAGGAGACCAGGGTCTTTCT | 206 |
| 27 | NDARGDKSTY | 25 | AACGACGCAAGAGGAGACAAATCCACATAC | 207 |
| 28 | VGLRGDLTGS | 26 | GTGGGGCTGAGAGGAGACTTGACGGGTTCG | 208 |
| 29 | EMRRGDLGTS | 27 | GAGATGAGGAGAGGAGACCTGGGGACGAGT | 209 |
| 30 | AMSRGDMGMA | 28 | GCGATGAGTAGAGGAGACATGGGGATGGCG | 210 |
| 31 | ATGRGDLGQA | 179 | GCAACGGGCAGAGGAGACTTAGGCCAAGCA | 211 |
| 32 | NVARGDQVNY | 29 | AACGTAGCAAGAGGAGACCAAGTTAACTAC | 212 |
| 33 | SIGRGDTGHM | 15 | AGCATCGGAAGAGGAGACACCGGCCACATG | 213 |
| 34 | ASVRGDLSGS | 30 | GCATCCGTAAGAGGAGACCTATCAGGCTCA | 214 |
| 35 | SAARGDTERL | 31 | AGCGCCGCGAGAGGAGACACTGAACGGCTA | 215 |
| 36 | AMSRGDMGMA | 28 | GCCATGTCAAGAGGAGACATGGGTATGGCT | 216 |
| 37 | SPSRGDQGRT | 9 | AGTCCATCGAGAGGAGACCAAGGACGCACT | 217 |
| 38 | VPGRGDLNTM | 32 | GTGCCTGGGAGAGGAGACCTTAATACTATG | 218 |
| 39 | GPGRGDQTTL | 178 | GGTCCTGGGAGAGGAGACCAAACGACCCTT | 219 |
| 40 | TSVRGDHGTL | 33 | ACATCGGTCAGAGGAGACCACGGAACATTG | 220 |
| 41 | TPSRGDLGQT | 34 | ACGCCTTCGAGAGGAGACCTTGGGCAGACT | 221 |
| 42 | AGHRGDTGVI | 35 | GCTGGACACAGAGGAGACACAGGAGTCATC | 222 |
| 43 | APARGDQGSM | 36 | GCGCCGGCGAGAGGAGACCAGGGTAGTATG | 223 |
| 44 | MSLRGDLNGS | 37 | ATGTCGTTGAGAGGAGACTTGAATGGGTCG | 224 |
| 45 | EAKRGDVHSI | 38 | GAGGCTAAGAGAGGAGACGTGCATTCTATT | 225 |
| 46 | SAQRGDVQAV | 39 | AGTGCTCAGAGAGGAGACGTGCAGGCGGTG | 226 |
| 47 | STARGDQGDR | 40 | AGTACGGCGAGAGGAGACCAGGGGGATAGG | 227 |
| 48 | QISRGDLGIN | 41 | CAGATTAGTAGAGGAGACCTGGGGATTAAT | 228 |
| 49 | TFTRGDMTMN | 42 | ACGTTCACAAGAGGAGACATGACGATGAAC | 229 |
| 50 | QEGRGDLNMR | 43 | CAAGAAGGAAGAGGAGACCTGAACATGAGG | 230 |
| 51 | AHARGDTSSL | 44 | GCACACGCAAGAGGAGACACCAGCTCCCTG | 231 |
| 52 | TNGRGDAGTL | 45 | ACGAATGGGAGAGGAGACGCTGGGACTCTG | 232 |
| 53 | AFGRGDQGQL | 46 | GCTTTCGGCAGAGGAGACCAAGGGCAACTA | 233 |
| 54 | ADGRGDRSSL | 47 | GCTGATGGGAGAGGAGACCGTTCGTCTCTG | 234 |
| 55 | DGRRGDGHSL | 48 | GACGGACGCAGAGGAGACGGACACAGCCTT | 235 |
| 56 | EKLRGDLHST | 49 | GAGAAGTTGAGAGGAGACCTTCATTCGACT | 236 |
| 57 | EARRGDASAM | 50 | GAGGCGCGTAGAGGAGACGCTTCGGCGATG | 237 |
| 58 | MGTRGDKMDF | 51 | ATGGGAACGAGAGGAGACAAAATGGACTTC | 238 |
| 59 | STSRGDRESY | 52 | TCGACTTCGAGAGGAGACCGGGAGTCGTAT | 239 |
| 60 | CQPRGDTTRC | 53 | TGTCAGCCGAGAGGAGACACGACTCGGTGT | 240 |
| 61 | MHTRGDKMDF | 54 | ATGCACACGAGAGGAGACAAAATGGACTTC | 241 |
| 62 | YSARGDTSGL | 55 | TACAGTGCCAGAGGAGACACAAGTGGCCTG | 242 |
| 63 | STMRGDHEKL | 56 | TCGACGATGAGAGGAGACCATGAGAAGTTG | 243 |
| 64 | TQMRGDGVTL | 57 | ACCCAAATGAGAGGAGACGGGGTCACACTA | 244 |
| 65 | TTRRGDMGDN | 58 | ACTACTCGTAGAGGAGACATGGGGGATAAT | 245 |
| 66 | ANGRGDRLEL | 59 | GCGAATGGTAGAGGAGACAGGCTGGAGTTG | 246 |
| 67 | AVSRGDRMEF | 8 | GCTGTGTCTAGAGGAGACAGGATGGAGTTT | 247 |
| 68 | ENQRGDLSGR | 60 | GAGAATCAGAGAGGAGACTTGTCGGGTCGG | 248 |
| 69 | STGRGDLGQA | 61 | TCGACTGGTAGAGGAGACCTGGGGCAGGCT | 249 |
| 70 | MHTRGDKMDF | 54 | ATGCATACTAGAGGAGACAAGATGGATTTT | 250 |
| 71 | SSARGDYSEV | 62 | AGTAGTGCGAGAGGAGACTATAGTGAGGTG | 251 |
| 72 | AFGRGDQGQL | 46 | GCGTTTGGGAGAGGAGACCAGGGTCAGCTT | 252 |
| 73 | STARGDAATY | 63 | AGTACGGCGAGAGGAGACGCGGCGACTTAT | 253 |
| 74 | VNTRGDTQKL | 64 | GTCAACACAAGAGGAGACACTCAAAAACTT | 254 |
| 75 | AMSRGDHASL | 65 | GCGATGAGTAGAGGAGACCATGCGTCGCTT | 255 |
| 76 | QAARGDVNKL | 66 | CAGGCTGCTAGAGGAGACGTTAATAAGCTG | 256 |
| 77 | RGTRGDTVEL | 67 | AGGGGTACGAGAGGAGACACGGTTGAGTTG | 257 |
| 78 | TTRRGDMGDN | 58 | ACCACCCGGAGAGGAGACATGGGAGACAAC | 258 |
| 79 | ASTRGDYAGV | 68 | GCTAGTACGAGAGGAGACTATGCGGGTGTT | 259 |
| 80 | DDRRGDKLPL | 69 | GACGACAGGAGAGGAGACAAACTGCCCCTT | 260 |
| 81 | SGSRGDLNAV | 70 | TCGGGGTCGAGAGGAGACCTGAATGCGGTG | 261 |
| 82 | EYRRGDQQIQ | 71 | GAGTATAGGAGAGGAGACCAGCAGATTCAG | 262 |
| 83 | TYVRGDRAEV | 72 | ACGTACGTCAGAGGAGACAGAGCAGAAGTG | 263 |
| 84 | QSLRGDLNGS | 73 | CAATCACTAAGAGGAGACTTGAACGGATCC | 264 |
| 85 | EGRRGDTTSL | 74 | GAGGGTCGGAGAGGAGACACGACGTCGTTG | 265 |
| 86 | AENRGDRSSL | 75 | GCGGAGAATAGAGGAGACCGGTCGAGTTTG | 266 |
| 87 | VAGRGDRSEL | 76 | GTGGCTGGGAGAGGAGACCGTAGTGAGCTG | 267 |
| 88 | DPARGDIGAR | 77 | GATCCGGCGAGAGGAGACATTGGTGCGCGT | 268 |
| 89 | EMRRGDLGTS | 27 | GAAATGCGCAGAGGAGACTTGGGAACTTCA | 269 |
| 90 | VWQRGDKMDM | 14 | GTTTGGCAGAGAGGAGACAAGATGGATATG | 270 |
| 91 | VIGRGDKALQ | 78 | GTGATTGGGAGAGGAGACAAGGCGCTTCAG | 271 |
| 92 | SFERGDKNSL | 79 | AGCTTCGAAAGAGGAGACAAAAACTCTCTT | 272 |
| 93 | QIARGDIASV | 80 | CAGATTGCTAGAGGAGACATTGCTAGTGTG | 273 |
| 94 | TISRGDLGGA | 81 | ACGATTAGTAGAGGAGACCTGGGTGGGGCT | 274 |
| 95 | AMIRGDMTHS | 82 | GCGATGATTAGAGGAGACATGACGCATAGT | 275 |
| 96 | TSVRGDHGTL | 33 | ACGAGTGTGAGAGGAGACCATGGGACGCTG | 276 |
| 97 | TMRRGDLNDS | 83 | ACGATGCGGAGAGGAGACCTGAATGATAGT | 277 |
| 98 | AYSRGDLGNH | 84 | GCCTACTCCAGAGGAGACCTAGGCAACCAC | 278 |
| 99 | EERRGDTHRL | 85 | GAGGAGCGGAGAGGAGACACTCATCGGCTG | 279 |
| 100 | SQLRGDAGTI | 86 | TCACAACTAAGAGGAGACGCGGGGACAATC | 280 |
| 101 | AMVRGDAHQL | 87 | GCGATGGTGAGAGGAGACGCGCATCAGCTG | 281 |
| 102 | TTSRGDLNSV | 88 | ACGACTAGTAGAGGAGACTTGAATTCGGTT | 282 |
| 103 | WEQRGDMIGK | 89 | TGGGAGCAGAGAGGAGACATGATTGGGAAG | 283 |
| 104 | YTTRGDLQSN | 90 | TACACCACCAGAGGAGACCTCCAATCAAAC | 284 |
| 105 | MSQRGDLSHQ | 91 | ATGTCGCAGAGAGGAGACCTTTCGCATCAG | 285 |
| 106 | ASTRGDTTHM | 92 | GCGAGTACTAGAGGAGACACGACGCATATG | 286 |
| 107 | SDTRGDTHRL | 93 | TCAGACACCAGAGGAGACACCCACAGACTG | 287 |
| 108 | TLGRGDQYTS | 94 | ACGCTGGGTAGAGGAGACCAGTATACGAGT | 288 |
| 109 | SYARGDVREV | 95 | TCGTATGCGAGAGGAGACGTTAGGGAGGTT | 289 |
| 110 | ETRRGDLSQN | 96 | GAGACTCGTAGAGGAGACCTGAGTCAGAAT | 290 |
| 111 | TIGRGDLMDK | 97 | ACAATCGGGAGAGGAGACCTGATGGACAAA | 291 |
| 112 | SYTRGDLSQN | 98 | AGTTACACCAGAGGAGACCTTAGCCAAAAC | 292 |
| 113 | SKLRGDASEI | 99 | TCGAAGCTGAGAGGAGACGCTAGTGAGATT | 293 |
| 114 | SMARGDKAEI | 100 | TCGATGGCGAGAGGAGACAAGGCGGAGATT | 294 |
| 115 | GAVRGDHSSL | 101 | GGAGCCGTCAGAGGAGACCACTCATCGTTA | 295 |
| 116 | METRGDMSNR | 102 | ATGGAGACGAGAGGAGACATGAGTAATCGT | 296 |
| 117 | AYTRGDKDAL | 103 | GCGTACACAAGAGGAGACAAAGACGCACTA | 297 |
| 118 | ENRRGDITQS | 104 | GAGAATCGGAGAGGAGACATTACGCAGTCG | 298 |
| 119 | STGRGDLGQA | 61 | TCCACGGGCAGAGGAGACCTTGGACAAGCA | 299 |
| 120 | GAVRGDHSSL | 101 | GGTGCTGTTAGAGGAGACCATAGTAGTCTT | 300 |
| 121 | SQLRGDAGTI | 86 | TCTCAGCTTAGAGGAGACGCTGGTACGATT | 301 |
| 122 | MLLRGDKNDF | 105 | ATGCTCCTAAGAGGAGACAAAAACGACTTC | 302 |
| 123 | KVQRGDASDW | 106 | AAGGTGCAGAGAGGAGACGCGAGTGATTGG | 303 |
| 124 | AFTRGDMQTA | 107 | GCGTTTACGAGAGGAGACATGCAGACTGCT | 304 |
| 125 | QPKRGDVQAQ | 108 | CAGCCTAAGAGAGGAGACGTTCAGGCTCAG | 305 |
| 126 | ASLRGDMGVS | 109 | GCTAGTCTGAGAGGAGACATGGGGGTGAGT | 306 |
| 127 | AYSRGDKESF | 110 | GCCTACTCCAGAGGAGACAAAGAATCGTTC | 307 |
| 128 | RFQRGDLTDA | 111 | CGGTTCCAAAGAGGAGACTTGACCGACGCT | 308 |
| 129 | SDARGDTMKL | 112 | TCTGATGCTAGAGGAGACACGATGAAGCTG | 309 |
| 130 | SGARGDTVSL | 113 | AGCGGGGCCAGAGGAGACACAGTAAGTCTC | 310 |
| 131 | DEKRGDQKHL | 114 | GATGAGAAGAGAGGAGACCAGAAGCATCTT | 311 |
| 132 | NGVRGDVQNF | 115 | AACGGAGTAAGAGGAGACGTCCAAAACTTC | 312 |
| 133 | MTLRGDLGGS | 116 | ATGACGTTGAGAGGAGACCTGGGTGGGTCG | 313 |
| 134 | EARRGDASAM | 50 | GAGGCTAGGAGAGGAGACGCTAGTGCGATG | 314 |
| 135 | GQERGDLGTR | 117 | GGGCAGGAGAGAGGAGACCTGGGGACTCGG | 315 |
| 136 | RGARGDLVDA | 118 | AGAGGCGCCAGAGGAGACCTAGTCGACGCT | 316 |
| 137 | QYSRGDHTDL | 119 | CAATACTCCAGAGGAGACCACACCGACCTT | 317 |
| 138 | MNTRGDVHAM | 120 | ATGAACACCAGAGGAGACGTGCACGCTATG | 318 |
| 139 | TVRRGDLATE | 121 | ACGGTACGGAGAGGAGACCTAGCAACCGAA | 319 |
| 140 | NIARGDAGQY | 16 | AATATTGCGAGAGGAGACGCTGGTCAGTAT | 320 |
| 141 | SVGRGDKADI | 122 | TCTGTTGGGAGAGGAGACAAGGCTGATATT | 321 |
| 142 | SHNRGDTGTM | 123 | TCACACAACAGAGGAGACACCGGAACCATG | 322 |
| 143 | TNTRGDKESV | 124 | ACGAACACCAGAGGAGACAAAGAATCAGTA | 323 |
| 144 | SIGRGDQYTI | 125 | TCGATTGGTAGAGGAGACCAGTATACGATT | 324 |
| 145 | QTSRGDAGSW | 126 | CAGACGAGTAGAGGAGACGCGGGGTCTTGG | 325 |
| 146 | AENRGDRSSL | 75 | GCTGAAAACAGAGGAGACCGAAGCAGCCTA | 326 |
| 147 | AISRGDVQSL | 127 | GCGATTTCGAGAGGAGACGTTCAGTCGTTG | 327 |
| 148 | NAQRGDHGQL | 128 | AACGCACAAAGAGGAGACCACGGGCAACTG | 328 |
| 149 | LNSRGDQASV | 129 | CTCAACTCGAGAGGAGACCAAGCCTCCGTC | 329 |
| 150 | SGNRGDIGTF | 130 | TCGGGAAACAGAGGAGACATAGGTACATTC | 330 |
| 151 | NVGRGDQATM | 131 | AACGTGGGAAGAGGAGACCAAGCCACAATG | 331 |
| 152 | SYSRGDTGRL | 132 | TCGTATTCTAGAGGAGACACTGGTCGGCTT | 332 |
| 153 | SHLRGDQALA | 133 | AGCCACTTAAGAGGAGACCAAGCGTTGGCC | 333 |
| 154 | YFARGDMTMN | 134 | TACTTCGCCAGAGGAGACATGACTATGAAC | 334 |
| 155 | QSQRGDLNPM | 135 | CAGTCGCAGAGAGGAGACCTTAATCCTATG | 335 |
| 156 | EQRRGDKTEL | 136 | GAGCAGAGGAGAGGAGACAAGACGGAGCTG | 336 |
| 157 | VSARGDAGQL | 137 | GTGTCTGCGAGAGGAGACGCGGGTCAGTTG | 337 |
| 158 | SPSRGDQGRT | 9 | TCTCCGTCTAGAGGAGACCAGGGGCGGACG | 338 |
| 159 | EASRGDKGTH | 138 | GAGGCTAGTAGAGGAGACAAGGGTACGCAT | 339 |
| 160 | LSTRGDMGMQ | 139 | CTGTCTACTAGAGGAGACATGGGTATGCAG | 340 |
| 161 | ANMRGDQLHT | 140 | GCGAATATGAGAGGAGACCAGCTGCATACT | 341 |
| 162 | ENTRGDLTHA | 141 | GAGAATACGAGAGGAGACCTTACTCATGCG | 342 |
| 163 | IERRGDSAGL | 142 | ATTGAGCGTAGAGGAGACTCGGCGGGGCTG | 343 |
| 164 | SAMRGDGVSL | 143 | AGTGCGATGAGAGGAGACGGAGTGAGCTTG | 344 |
| 165 | TVSRGDQGLS | 144 | ACGGTTTCGAGAGGAGACCAGGGGTTGTCT | 345 |
| 166 | SIVRGDLNST | 145 | TCGATCGTCAGAGGAGACCTGAACTCCACG | 346 |
| 167 | DVARGDKTNF | 146 | GACGTAGCGAGAGGAGACAAAACAAACTTC | 347 |
| 168 | WSQRGDLSGT | 147 | TGGAGCCAAAGAGGAGACCTGAGTGGAACC | 348 |
| 169 | LNQRGDVSNM | 148 | CTGAACCAAAGAGGAGACGTTTCCAACATG | 349 |
| 170 | AALRGDLSGS | 149 | GCCGCCCTAAGAGGAGACCTATCCGGCTCA | 350 |
| 171 | RVARGDITDI | 150 | AGGGTTGCGAGAGGAGACATTACGGATATT | 351 |
| 172 | STNRGDLNQV | 151 | TCGACGAACAGAGGAGACCTCAACCAAGTT | 352 |
| 173 | DSRRGDSVSL | 152 | GATTCGCGGAGAGGAGACTCTGTGAGTCTT | 353 |
| 174 | SVGRGDQSQM | 153 | AGTGTTGGGAGAGGAGACCAGTCTCAGATG | 354 |
| 175 | SNTRGDTNSL | 154 | TCCAACACGAGAGGAGACACAAACTCCCTA | 355 |
| 176 | DNRRGDGTTM | 155 | GATAATCGGAGAGGAGACGGTACGACTATG | 356 |
| 177 | TPSRGDQGRL | 156 | ACGCCTTCGAGAGGAGACCAAGGAAGACTA | 357 |
| 178 | NYSRGDSMTL | 157 | AACTACAGCAGAGGAGACTCAATGACGCTT | 358 |
| 179 | MQARGDAGTL | 158 | ATGCAGGCTAGAGGAGACGCTGGGACTCTG | 359 |
| 180 | TQSRGDLSGA | 159 | ACGCAGTCGAGAGGAGACTTGTCTGGTGCG | 360 |
| 181 | FAQRGDLTGV | 160 | TTTGCGCAGAGAGGAGACTTGACTGGGGTT | 361 |
| 182 | APVRGDLIGT | 161 | GCGCCGGTGAGAGGAGACCTGATTGGTACG | 362 |
| 183 | SNTRGDTNSL | 154 | TCGAATACTAGAGGAGACACGAATAGTTTG | 363 |
| 184 | EPKRGDLSNT | 162 | GAGCCGAAGAGAGGAGACTTGAGTAATACG | 364 |
| 185 | SVGRGDTYPL | 163 | TCGGTTGGGAGAGGAGACACTTATCCTCTG | 365 |
| 186 | QTARGDMTGH | 164 | CAAACAGCCAGAGGAGACATGACTGGCCAC | 366 |
| 187 | AGARGDLENR | 165 | GCTGGTGCGAGAGGAGACTTGGAGAATCGG | 367 |
| 188 | MSTRGDLNNV | 166 | ATGAGCACTAGAGGAGACCTAAACAACGTC | 368 |
| 189 | EKQRGDLNSM | 167 | GAAAAACAAAGAGGAGACCTCAACAGCATG | 369 |
| 190 | IKTRGDLGHE | 168 | ATTAAGACGAGAGGAGACCTTGGTCATGAG | 370 |
| 191 | IGRRGDMELS | 169 | ATTGGGAGGAGAGGAGACATGGAGTTGTCT | 371 |
| 192 | TTGRGDLKEV | 170 | ACTACGGGTAGAGGAGACTTGAAGGAGGTT | 372 |
| 193 | TDKRGDQNTV | 171 | ACGGATAAGAGAGGAGACCAGAATACTGTG | 373 |
| 194 | SNVRGDREAV | 172 | TCGAATGTTAGAGGAGACCGGGAGGCGGTT | 374 |
| 195 | SVGRGDQSQM | 153 | TCAGTCGGCAGAGGAGACCAAAGTCAAATG | 375 |
| 196 | YANRGDLSHQ | 173 | TACGCCAACAGAGGAGACCTGAGTCACCAA | 376 |
| 197 | QISRGDLGIN | 41 | CAAATATCGAGAGGAGACCTAGGTATCAAC | 377 |
| 198 | SMMRGDAGQL | 174 | TCGATGATGAGAGGAGACGCTGGGCAGTTG | 378 |
| 199 | EARRGDSQGL | 175 | GAGGCGCGGAGAGGAGACAGTCAGGGGCTG | 379 |
| 200 | MPTRGDQTKF | 176 | ATGCCCACAAGAGGAGACCAAACCAAATTC | 380 |

TABLE 2
Enhanced MyoAAV (eMyoAAV) Capsid Variants

| | | SEQ | | SEQ |
|---|---|---|---|---|
| | | ID | | ID |
| RANK | N-MER INSERT | NO.: | ENCODING SEQUENCE | NO.: |
| 1 | SNSRGDYNSL | 381 | TCAAACTCCAGAGGAGACTACAACTCCTTG | 557 |
| 2 | SSARGDRDYL | 397 | TCGTCGGCTAGAGGAGACCGTGATTATCTG | 558 |
| 3 | STVRGDYTSV | 398 | AGCACGGTCAGAGGAGACTACACGTCCGTG | 559 |
| 4 | QERRGDYTSM | 382 | CAGGAGCGGAGAGGAGACTATACGAGTATG | 560 |
| 5 | NGGRGDTTHF | 386 | AACGGCGGGAGAGGAGACACGACGCACTTC | 561 |
| 6 | ASTRGDHGVL | 383 | GCGAGTACTAGAGGAGACCATGGTGTGTTG | 562 |
| 7 | SYQRGDQHNM | 399 | TCGTACCAAAGAGGAGACCAACACAACATG | 563 |
| 8 | NDTRGDRSYM | 400 | AACGACACAAGAGGAGACCGATCTTACATG | 564 |
| 9 | ENRRGDFNNT | 384 | GAAAACAGGAGAGGAGACTTCAACAACACT | 565 |
| 10 | SGSRGDLSGH | 401 | TCCGGCTCGAGAGGAGACTTGTCAGGACAC | 566 |
| 11 | ENRRGDITQS | 393 | GAGAATCGGAGAGGAGACATTACGCAGTCG | 567 |
| 12 | DKPRGDRQLL | 402 | GATAAGCCGAGAGGAGACCGGCAGTTGTTG | 568 |
| 13 | EERRGDTHRL | 392 | GAGGAGCGGAGAGGAGACACTCATCGGCTG | 569 |
| 14 | AMIRGDMTHS | 391 | GCGATGATTAGAGGAGACATGACGCATAGT | 570 |
| 15 | SNNRGDYTTM | 403 | TCAAACAACAGAGGAGACTACACGACAATG | 571 |
| 16 | QGGRGDLSTL | 404 | CAGGGTGGGAGAGGAGACTTGTCGACGTTG | 572 |
| 17 | SKDRGDYQTL | 405 | TCGAAGGATAGAGGAGACTATCAGACTCTT | 573 |
| 18 | ATARGDQMKL | 406 | GCTACGGCGAGAGGAGACCAGATGAAGCTG | 574 |
| 19 | QHMRGDLTGA | 407 | CAGCATATGAGAGGAGACTTGACTGGTGCG | 575 |
| 20 | DGTRGDLGRY | 408 | GACGGCACGAGAGGAGACCTTGGAAGGTAC | 576 |
| 21 | DSRRGDYANL | 409 | GACTCTCGGAGAGGAGACTACGCAAACCTC | 577 |
| 22 | SAMRGDYTST | 410 | TCGGCGATGAGAGGAGACTATACTAGTACT | 578 |
| 23 | ENRRGDAQLL | 411 | GAGAATCGGAGAGGAGACGCGCAGCTGCTG | 579 |
| 24 | TTLRGDHSQL | 412 | ACGACGCTGAGAGGAGACCATAGTCAGCTT | 580 |
| 25 | DAGRGDMHRM | 413 | GACGCAGGGAGAGGAGACATGCACCGTATG | 581 |
| 26 | QHTRGDLSST | 414 | CAGCATACGAGAGGAGACTTGAGTTCTACT | 582 |
| 27 | QVYRGDRESV | 415 | CAAGTTTACAGAGGAGACAGGGAATCCGTG | 583 |
| 28 | SSQRGDLAGT | 416 | TCGAGTCAGAGAGGAGACTTGGCTGGTACT | 584 |
| 29 | TDVRGDRGTF | 417 | ACGGATGTTAGAGGAGACAGGGGGACGTTT | 585 |
| 30 | SSVRGDREVT | 418 | TCGTCTGTGAGAGGAGACCGGGAGGTGACG | 586 |
| 31 | ENRRGDLTNA | 419 | GAGAATAGGAGAGGAGACCTGACTAATGCG | 587 |
| 32 | SYARGDVHSI | 420 | TCGTATGCGAGAGGAGACGTTCATTCTATT | 588 |
| 33 | DSSRGDLNLR | 421 | GACAGCTCAAGAGGAGACCTCAACCTCCGG | 589 |
| 34 | SNARGDYSNM | 422 | TCGAACGCGAGAGGAGACTACTCAAACATG | 590 |
| 35 | SSVRGDHSVL | 423 | TCGTCTGTGAGAGGAGACCATTCGGTGCTT | 591 |
| 36 | SAFRGDLHAT | 424 | TCGGCTTTTAGAGGAGACCTGCATGCTACT | 592 |
| 37 | SAMRGDYTST | 410 | TCCGCCATGAGAGGAGACTACACAAGCACG | 593 |
| 38 | NAQRGDHGQL | 394 | AACGCACAAAGAGGAGACCACGGGCAACTG | 594 |
| 39 | SDRRGDQLLY | 425 | TCGGATCGGAGAGGAGACCAGCTGTTGTAT | 595 |
| 40 | SGVRGDRLAV | 426 | AGCGGCGTAAGAGGAGACCGCCTAGCCGTA | 596 |
| 41 | ASLRGDLSST | 427 | GCCAGCCTAAGAGGAGACCTCAGCTCAACG | 597 |
| 42 | TNGRGDLHGM | 428 | ACGAACGGAAGAGGAGACCTTCACGGAATG | 598 |
| 43 | TNTRGDHGML | 429 | ACGAATACGAGAGGAGACCATGGGATGCTG | 599 |
| 44 | SSARGDRDYL | 397 | AGTTCCGCCAGAGGAGACCGGGACTACCTC | 600 |
| 45 | EDRRGDLLRT | 430 | GAGGATCGGAGAGGAGACCTTTTGAGGACT | 601 |
| 46 | SSLRGDLLHS | 431 | TCGTCGCTGAGAGGAGACCTGTTGCATTCT | 602 |
| 47 | ENRRGDLTNA | 419 | GAAAACCGTAGAGGAGACCTCACGAACGCT | 603 |
| 48 | DGRRGDRESI | 432 | GACGGCAGGAGAGGAGACCGGGAATCGATC | 604 |
| 49 | TGTRGDTMTW | 433 | ACTGGAACCAGAGGAGACACAATGACATGG | 605 |
| 50 | STVRGDYTSV | 398 | TCGACTGTTAGAGGAGACTATACTTCTGTT | 606 |
| 51 | SATRGDHNVL | 434 | TCGGCAACGAGAGGAGACCACAACGTACTG | 607 |
| 52 | TDRRGDSGTL | 435 | ACGGATCGGAGAGGAGACAGTGGTACTCTG | 608 |
| 53 | TSARGDLIHT | 436 | ACGAGTGCTAGAGGAGACCTGATTCATACG | 609 |
| 54 | SGVRGDRLAV | 426 | AGTGGGGTGAGAGGAGACCGGCTGGCGGTG | 610 |
| 55 | AVERGDRLML | 437 | GCGGTTGAGAGAGGAGACCGTCTTATGCTG | 611 |
| 56 | DRSRGDTHVL | 438 | GACAGGTCGAGAGGAGACACGCACGTACTC | 612 |
| 57 | ESTRGDLRVV | 439 | GAGTCGACGAGAGGAGACCTTCGTGTTGTG | 613 |
| 58 | TTIRGDYREL | 440 | ACGACGATTAGAGGAGACTATCGTGAGTTG | 614 |
| 59 | TFIRGDLAGA | 441 | ACGTTCATCAGAGGAGACCTGGCAGGTGCG | 615 |
| 60 | MTSRGDLTTY | 442 | ATGACGAGCAGAGGAGACTTGACAACATAC | 616 |
| 61 | ADRRGDQLYS | 443 | GCGGATCGTAGAGGAGACCAGCTTTATTCG | 617 |
| 62 | AHMRGDLGGT | 385 | GCACACATGAGAGGAGACCTAGGCGGCACG | 618 |
| 63 | GYVRGDLGQH | 444 | GGGTACGTCAGAGGAGACCTGGGGCAACAC | 619 |
| 64 | DASRGDRTSL | 445 | GATGCGAGTAGAGGAGACAGGACTAGTTTG | 620 |
| 65 | TEVRGDRSQV | 446 | ACGGAGGTGAGAGGAGACAGGTCGCAGGTT | 621 |
| 66 | SHLRGDQALA | 395 | TCGCATCTGAGAGGAGACCAGGCTCTTGCG | 622 |
| 67 | SEVRGDRLSI | 447 | TCGGAGGTGAGAGGAGACCGGTTGAGTATT | 623 |
| 68 | SAARGDTERL | 387 | AGCGCCGCGAGAGGAGACACTGAACGGCTA | 624 |
| 69 | SDRRGDLSGA | 448 | TCAGACCGTAGAGGAGACCTGTCGGGCGCT | 625 |
| 70 | SVTRGDRVVI | 449 | AGCGTCACAAGAGGAGACAGAGTAGTCATC | 626 |
| 71 | SEGRGDRMAL | 450 | TCGGAGGGGAGAGGAGACAGGATGGCTCTT | 627 |
| 72 | SSTRGDHLSL | 451 | TCGTCTACTAGAGGAGACCATCTTTCGCTG | 628 |
| 73 | AMIRGDMTHS | 391 | GCTATGATCAGAGGAGACATGACCCACTCC | 629 |
| 74 | SSTRGDQIYV | 452 | TCGTCGACGAGAGGAGACCAGATTTATGTG | 630 |
| 75 | EERRGDTHRL | 392 | GAGGAGCGGAGAGGAGACACTCATCGGTTG | 631 |
| 76 | SNSRGDYNSL | 381 | TCGAATTCGAGAGGAGACTATAATAGTCTT | 632 |
| 77 | SAQRDRMGSP | 453 | TCCGCACAAAGGGATCGTATGGGTTCTCCG | 633 |
| 78 | TGTRGDIATF | 454 | ACGGGTACGAGAGGAGACATTGCGACTTTT | 634 |
| 79 | SMQRGDLLST | 455 | TCGATGCAGAGAGGAGACCTGCTTTCTACT | 635 |
| 80 | EDVRGDRSKL | 456 | GAGGATGTGAGAGGAGACAGGAGTAAGCTT | 636 |
| 81 | AHMRGDLGGT | 385 | GCGCATATGAGAGGAGACTTGGGGGGGACT | 637 |
| 82 | TSVRGDQSQY | 457 | ACGAGTGTGAGAGGAGACCAGAGTCAGTAT | 638 |
| 83 | ADRRGDTGLF | 458 | GCGGATCGGAGAGGAGACACGGGGTTGTTT | 639 |
| 84 | NTYRGDRDSL | 459 | AACACTTACAGAGGAGACCGAGACAGCCTG | 640 |
| 85 | TDVRGDRGTF | 417 | ACGGATGTTAGAGGAGACCGGGGGACGTTT | 641 |

| | | | | |
|---|---|---|---|---|
| 86 | SRGRGDLNDL | 460 | TCTCGTGGGAGAGGAGACCTGAATGATTTG | 642 |
| 87 | SNVRGDREAV | 396 | AGCAACGTAAGAGGAGACAGGGAAGCCGTC | 643 |
| 88 | MDRRGDASIL | 461 | ATGGACAGGAGAGGAGACGCCTCCATACTG | 644 |
| 89 | SIWRGDRTEV | 462 | TCGATATGGAGAGGAGACCGTACAGAAGTC | 645 |
| 90 | NSQRGDYSGM | 463 | AACTCACAAAGAGGAGACTACAGTGGAATG | 646 |
| 91 | SSVRGDIGGI | 464 | TCTAGCGTAAGAGGAGACATCGGGGGGATC | 647 |
| 92 | ENQRGDLSGR | 390 | GAGAATCAGAGAGGAGACTTGTCGGGTCGG | 648 |
| 93 | EIRRGDLLAQ | 465 | GAGATTCGGAGAGGAGACCTTCTGGCTCAG | 649 |
| 94 | SNIRGDHAVM | 466 | TCGAACATCAGAGGAGACCACGCAGTCATG | 650 |
| 95 | LDTRGDRSQV | 467 | CTGGATACGAGAGGAGACCGGAGTCAGGTT | 651 |
| 96 | ANTRGDHQNF | 468 | GCGAATACGAGAGGAGACCATCAGAATTTT | 652 |
| 97 | QVYRGDRESV | 415 | CAGGTGTATAGAGGAGACCGTGAGTCTGTT | 653 |
| 98 | TSVRGDLSMN | 469 | ACGTCGGTGAGAGGAGACCTTTCGATGAAT | 654 |
| 99 | ASTRGDHGVL | 383 | GCCTCAACGAGAGGAGACCACGGAGTTCTC | 655 |
| 100 | TTTRGDYLNT | 470 | ACGACGACGAGAGGAGACTATCTTAATACG | 656 |
| 101 | AERRGDIKEY | 471 | GCGGAGAGGAGAGGAGACATTAAGGAGTAT | 657 |
| 102 | SHARGDLGST | 472 | TCGCATGCTAGAGGAGACTTGGGTTCGACT | 658 |
| 103 | TSQRGDYVSL | 473 | ACGTCTCAGAGAGGAGACTATGTTTCTCTT | 659 |
| 104 | GPGRGDQTTL | 556 | GGGCCGGGTAGAGGAGACCAGACTACGTTG | 660 |
| 105 | VNVRGDRGEV | 474 | GTGAATGTGAGAGGAGACCGTGGTGAGGTG | 661 |
| 106 | SYRRGDHDQL | 475 | TCGTATCGTAGAGGAGACCATGATCAGCTT | 662 |
| 107 | SGTRGDRVDL | 476 | AGCGGCACAAGAGGAGACAGGGTAGACCTC | 663 |
| 108 | SHFRGDLHTS | 477 | TCCCACTTCAGAGGAGACTTGCACACGTCC | 664 |
| 109 | SDRRGDLSVP | 478 | TCGGATCGTAGAGGAGACCTTTCGGTGCCT | 665 |
| 110 | SGTRGDTLIL | 479 | AGCGGCACCAGAGGAGACACCCTTATACTC | 666 |
| 111 | ENTRGDLHGK | 480 | GAAAACACCAGAGGAGACCTCCACGGCAAA | 667 |
| 112 | DRARGDTHVL | 481 | GATCGGGCGAGAGGAGACACGCATGTGCTG | 668 |
| 113 | ASGRGDIAGL | 482 | GCGTCTGGTAGAGGAGACATTGCGGGGCTT | 669 |
| 114 | EGRRGDLFQA | 483 | GAGGGTCGTAGAGGAGACCTGTTTCAGGCG | 670 |
| 115 | RSERGDRLEI | 484 | AGATCCGAAAGAGGAGACAGGCTAGAAATC | 671 |
| 116 | AAYRGDAHVL | 485 | GCGGCCTACAGAGGAGACGCACACGTGCTC | 672 |
| 117 | NDSRGDQHRL | 486 | AACGACTCGAGAGGAGACCAACACCGATTG | 673 |
| 118 | SSARGDHSQL | 487 | TCATCGGCAAGAGGAGACCACTCTCAACTG | 674 |
| 119 | TNERGDRLSI | 488 | ACGAATGAGAGAGGAGACCGTCTTTCGATT | 675 |
| 120 | SNSRGDYTSV | 489 | AGCAACTCCAGAGGAGACTACACCAGTGTC | 676 |
| 121 | NVTRGDHTVM | 490 | AACGTCACAAGAGGAGACCACACCGTTATG | 677 |
| 122 | TMVRGDVKGL | 491 | ACGATGGTGAGAGGAGACGTTAAGGGGCTT | 678 |
| 123 | TGTRGDIATF | 454 | ACCGGGACAAGAGGAGACATAGCCACGTTC | 679 |
| 124 | VQLRGDLAST | 492 | GTGCAGCTGAGAGGAGACTTGGCTTCTACT | 680 |
| 125 | MNRRGDYSEQ | 493 | ATGAACCGGAGAGGAGACTACTCGGAACAA | 681 |
| 126 | ANTRGDLSPV | 494 | GCGAATACTAGAGGAGACCTGAGTCCGGTT | 682 |
| 127 | TGMRGDAGSL | 495 | ACGGGTATGAGAGGAGACGCTGGGAGTCTT | 683 |
| 128 | LDRRGDESSA | 496 | CTAGACCGCAGAGGAGACTTCAGCTCCGCG | 684 |
| 129 | MYHRGDMTSV | 497 | ATGTACCACAGAGGAGACATGACTTCGGTG | 685 |
| 130 | GSMRGDLHST | 498 | GGGTCGATGAGAGGAGACTTGCATTCGACG | 686 |
| 131 | SSSRGDFSSV | 499 | TCGTCGTCCAGAGGAGACTTCAGCTCTGTT | 687 |
| 132 | TGTRGDLHTY | 500 | ACGGGTACGAGAGGAGACTTGCATACTTAT | 688 |
| 133 | YTQRGDLATV | 501 | TACACCCAAAGAGGAGACCTTGCCACCGTC | 689 |
| 134 | TDVRGDRMYV | 502 | ACGGATGTTAGAGGAGACCGGATGTATGTG | 690 |
| 135 | TSVRGDHGTL | 388 | ACATCGGTCAGAGGAGACCACGGAACATTG | 691 |
| 136 | SNSRGDLSVN | 503 | TCGAACTCCAGAGGAGACCTATCGGTAAAC | 692 |
| 137 | QSGRGDQMTL | 504 | CAGTCTGGGAGAGGAGACCAGATGACGTTG | 693 |
| 138 | MNTRGDLQQS | 505 | ATGAACACCAGAGGAGACTTGCAACAATCG | 694 |
| 139 | AGSRGDLQTV | 506 | GCGGGATCAAGAGGAGACCTACAAACGGTG | 695 |
| 140 | SNMRGDQTYV | 507 | TCGAACATGAGAGGAGACCAAACTTACGTC | 696 |
| 141 | TPSRGDMSNV | 508 | ACGCCAAGTAGAGGAGACATGTCCAACGTC | 697 |
| 142 | NSTRGDLHMA | 509 | AACAGCACAAGAGGAGACCTACACATGGCA | 698 |
| 143 | ANTRGDLSVS | 510 | GCTAACACCAGAGGAGACCTCTCAGTCAGT | 699 |
| 144 | TDRRGDTGLL | 511 | ACCGACCGAAGAGGAGACACCGGCTTACTG | 700 |
| 145 | DGVRGDRATW | 512 | GACGGCGTAAGAGGAGACCGTGCAACATGG | 701 |
| 146 | ENRRGDFNNT | 384 | GAGAATCGTAGAGGAGACTTTAATAATACG | 702 |
| 147 | EPSRGDRMVL | 513 | GAGCCTAGTAGAGGAGACAGGATGGTGTTG | 703 |
| 148 | SGVRGDRIII | 514 | AGCGGCGTTAGAGGAGACCGGATCATAATC | 704 |
| 149 | TSMRGDYTHV | 515 | ACGTCGATGAGAGGAGACTATACGCATGTT | 705 |
| 150 | ASSRGDLTVI | 516 | GCGTCGAGTAGAGGAGACTTGACTGTTATT | 706 |
| 151 | ETRRGDLSLQ | 517 | GAGACGCGTAGAGGAGACCTGTCGCTTCAG | 707 |
| 152 | NNARGDQSMH | 518 | AACAACGCAAGAGGAGACCAATCAATGCAC | 708 |
| 153 | TDRRGDSSAL | 519 | ACGGATAGGAGAGGAGACTCTAGTGCGCTT | 709 |
| 154 | SSQRGDLTGV | 520 | TCAAGCCAAAGAGGAGACTTGACAGGTGTG | 710 |
| 155 | QTARGDVMVH | 521 | CAGACGGCTAGAGGAGACGTTATGGTTCAT | 711 |
| 156 | VSQRGDLNAV | 522 | GTGTCGCAGAGAGGAGACCTGAATGCTGTT | 712 |
| 157 | TMTRGDLAAN | 523 | ACGATGACCAGAGGAGACCTCGCTGCTAAC | 713 |
| 158 | NASRGDHSSL | 524 | AATGCTTCGAGAGGAGACCATTCGTCGTTG | 714 |
| 159 | SNTRGDMGLT | 525 | TCAAACACCAGAGGAGACATGGGCCTCACG | 715 |
| 160 | MYSRGDTHSL | 526 | ATGTATTCGAGAGGAGACACTCATAGTCTG | 716 |
| 161 | ADQRGDRAPL | 527 | GCGGATCAGAGAGGAGACAGGGCTCCGCTT | 717 |
| 162 | QERRGDYTSM | 382 | CAAGAAAGGAGAGGAGACTACACCTCTATG | 718 |
| 163 | SQNRGDLANT | 528 | TCCCAAAACAGAGGAGACCTAGCCAACACG | 719 |
| 164 | EKLRGDLHST | 389 | GAGAAGTTGAGAGGAGACCTTCATTCGACT | 720 |
| 165 | QGGRGDLSTL | 404 | CAAGGCGGAAGAGGAGACCTGAGTACACTG | 721 |
| 166 | SNRRGDTEMQ | 529 | TCGAATAGGAGAGGAGACACTGAGATGCAG | 722 |
| 167 | SGSRGDVSAL | 530 | TCGGGTAGTAGAGGAGACGTGAGTGCTTTG | 723 |
| 168 | ESTRGDRGTL | 531 | GAGTCGACGAGAGGAGACCGTGGTACGCTG | 724 |
| 169 | SAVRGDAALH | 532 | TCGGCGGTGAGAGGAGACGCGGCGCTTCAT | 725 |
| 170 | LSSRGDVNRL | 533 | CTGAGCAGCAGAGGAGACGTTAACCGCCTT | 726 |
| 171 | EQRRGDIQTI | 534 | GAGCAGAGGAGAGGAGACATTCAGACTATT | 727 |
| 172 | AAYRGDAHVL | 485 | GCTGCGTATAGAGGAGACGCGCATGTTCTT | 728 |
| 173 | SPVRGDHGAL | 535 | TCGCCGGTGAGAGGAGACCATGGGGCTTTG | 729 |
| 174 | DSRRGDYANL | 409 | GATTCGAGGAGAGGAGACTATGCGAATCTG | 730 |
| 175 | LSRRGDYQEL | 536 | TTGAGTCGGAGAGGAGACTATCAGGAGTTG | 731 |
| 176 | SYARGDVHSI | 420 | TCCTACGCAAGAGGAGACGTCCACTCCATC | 732 |
| 177 | DSRRGDASYH | 537 | GACTCACGCAGAGGAGACGCGTCGTACCAC | 733 |
| 178 | NHQRGDLSSS | 538 | AACCACCAAAGAGGAGACCTGAGCTCGAGT | 734 |
| 179 | SSTRGDHLSL | 451 | TCCTCAACGAGAGGAGACCACCTGTCTTTG | 735 |
| 180 | SWNRGDISGL | 539 | TCGTGGAACAGAGGAGACATATCTGGCCTT | 736 |
| 181 | TGTRGDLGTM | 540 | ACGGGTACTAGAGGAGACCTGGGGACGATG | 737 |
| 182 | QQLRGDTHTL | 541 | CAGCAGCTTAGAGGAGACACGCATACTCTT | 738 |
| 183 | AGLRGDRDSL | 542 | GCGGGGTTGAGAGGAGACCGTGATTCGCTT | 739 |
| 184 | QVYRGDRDQL | 543 | CAGGTTTATAGAGGAGACCGGGATCAGTTG | 740 |
| 185 | AGVRGDRVTI | 544 | GCAGGAGTAAGAGGAGACAGAGTGACCATC | 741 |
| 186 | NSARGDLLHS | 545 | AACTCAGCCAGAGGAGACCTTCTGCACTCC | 742 |
| 187 | NHSRGDLTGV | 546 | AACCACAGTAGAGGAGACCTGACAGGCGTT | 743 |
| 188 | NAYRGDTSAF | 547 | AACGCTTACAGAGGAGACACATCCGCGTTC | 744 |
| 189 | SANRGDIMHT | 548 | TCGGCTAACAGAGGAGACATAATGCACACG | 745 |
| 190 | QSARGDLVSY | 549 | CAATCCGCCAGAGGAGACCTCGTCAGTTAC | 746 |
| 191 | DSRRGDASYH | 537 | GATAGTAGGAGAGGAGACGCTAGTTATCAT | 747 |
| 192 | SFVRGDVRTL | 550 | TCGTTTGTGAGAGGAGACGTTCGTACGCTG | 748 |
| 193 | SDMRGDRSVY | 551 | AGCGACATGAGAGGAGACCGATCTGTGTAC | 749 |
| 194 | SGIRGDRYPI | 552 | TCCGGAATCAGAGGAGACCGCTACCCAATA | 750 |
| 195 | SNVRGDMAHS | 553 | TCCAACGTCAGAGGAGACATGGCTCACAGC | 751 |
| 196 | TEVRGDRSQV | 446 | ACTGAGGTTAGAGGAGACCGTTCGCAGGTT | 752 |
| 197 | ASSRGDLQQT | 554 | GCGAGCAGCAGAGGAGACCTGCAACAAACG | 753 |
| 198 | QSARGDLVSY | 549 | CAGTCTGCTAGAGGAGACCTTGTGTCTTAT | 754 |
| 199 | DASRGDRTSL | 445 | GACGCATCTAGAGGAGACCGAACATCCCTC | 755 |
| 200 | NSVRGDLNQT | 555 | AACAGTGTCAGAGGAGACCTCAACCAAACT | 756 |

TABLE 3
Top Ranking Skeletal Muscle Specific
n-mer inserts and/or RGD Motifs

| Variant | SEQ ID NO.: |
|---|---|
| TTGRGDQARL | 757 |
| TTLRGDHSQL | 758 |
| TNLRGDYQLT | 759 |
| STSRGDYVTH | 760 |
| MPARGDLVQT | 761 |
| SGVRGDRLAV | 762 |
| SNERGDQHRL | 763 |
| STLRGDYAVS | 764 |
| SNARGDIMTT | 765 |
| EASRGDLQMR | 766 |
| TYARGDLSGH | 767 |
| DSSRGDRSSV | 768 |
| SQSRGDLVPL | 769 |
| SATRGDYSIL | 770 |
| QATRGDLQHT | 771 |
| HDDRGDRRML | 772 |
| SSHRGDLLAT | 773 |
| SYQRGDQHNM | 774 |
| SHQRGDYNTL | 775 |
| MDQRGDMSVV | 776 |
| QIARGDLHVP | 777 |
| TYTRGDQHMM | 778 |
| DASRGDRTSL | 779 |
| SDRRGDLHPQ | 780 |
| SATRGDHSTL | 781 |
| AYQRGDFTSV | 782 |
| TGNRGDLQSV | 783 |
| AWRRGDLENA | 784 |
| NAARGDHGLL | 785 |
| SYSRGDHQAL | 786 |
| DGVRGDRTVV | 787 |
| SSERGDKMST | 788 |
| TSMRGDYTHV | 789 |
| SGVRGDRIII | 790 |
| ENRRGDAQLL | 791 |
| TVYRGDREQH | 792 |
| LDRRGDLLNS | 793 |
| ESVRGDLRST | 794 |
| DKYRGDQRGL | 795 |
| TDRRGDAHIL | 796 |
| SQTRGDTSLM | 797 |
| DATRGDTRML | 798 |
| SMQRGDLTHA | 799 |
| SGTRGDTLIL | 800 |
| ANTRGDHQNF | 801 |
| TSQRGDHTQL | 802 |
| SHWRGDTSSL | 803 |
| ISGRGDRQEL | 804 |
| SVLRGDIGTI | 805 |
| QDSRGDLSGK | 806 |
| KPIRGDGAEL | 807 |
| TYMRGDLQHT | 808 |
| TELRGDLSST | 809 |
| SAMRGDHAVL | 810 |
| SWVRGDASTL | 811 |
| QYARGDPHAL | 812 |
| STNRGDIVVH | 813 |
| VNIRGDLHQT | 814 |
| REYRGDSSIL | 815 |
| STLRGDGSMM | 816 |
| SSQRGDITAH | 817 |
| TEFRGDRSTV | 818 |
| TSTRGDYVTV | 819 |
| NHLRGDVSAI | 820 |
| LERRGDLVST | 821 |
| NEYRGDHSHP | 822 |
| NWQRGDTSTL | 823 |
| TDSRGDRTVL | 824 |
| YAVRGDQHTI | 825 |
| FGVRGDTRDL | 826 |
| SSARGDTGML | 827 |
| SAMRGDQMGQ | 828 |
| SHTRGDLVTT | 829 |
| VTTRGDTAIL | 830 |
| EPTRGDRHSI | 831 |
| GSQRGDRLVL | 832 |
| QHYRGDLSTT | 833 |
| METRGDRQQL | 834 |
| EALRGDKVVL | 835 |
| NAVRGDQSYV | 836 |
| SPLRGDNRAL | 837 |
| ENRRGDTHAL | 838 |
| NSQRGDLTGT | 839 |
| QYAPSQGTV | 840 |
| DNSRGDHSRL | 841 |
| NTHRGDYASI | 842 |
| HTTRGDQQPL | 843 |
| EWRRGDQTAL | 844 |
| TAVRGDQTTV | 845 |
| EYLRGDRISQ | 846 |
| SRYRGDQETI | 847 |
| ASLRGDTVTL | 848 |
| VGVRGDSHVL | 849 |
| TNSRGDHTSL | 850 |
| VDQRGDQRAL | 851 |
| ASVRGDRLDV | 852 |
| TSYRGDLSSN | 853 |
| TSDRGDSSNR | 854 |
| SYMRGDLDMR | 855 |
| TDVRGDRSMI | 856 |
| SYVRGDSSTV | 857 |
| QVYRGDRESV | 858 |
| TDRRGDAVSF | 859 |
| TNSRGDLHTT | 860 |
| EAIRGDFKTL | 861 |
| TGIRGDTAGL | 862 |
| TSSRGDVQSL | 863 |
| SSRRGDTNEL | 864 |
| SNARGDHTPL | 865 |
| SELRGDRSPY | 866 |
| VHARGDTSIM | 867 |
| SALRGDHGTL | 868 |
| QTGRGDIAGM | 869 |
| TVARGDQIRL | 870 |
| TNSRGDSVAL | 871 |
| QNARGDSAVI | 872 |
| HYLRGDTTTL | 873 |
| ANARGDLQNT | 874 |
| QNIRGDIRTV | 875 |
| AALRGDHGQM | 876 |
| TTDRGDLSAR | 877 |
| LNVRGDLVHT | 878 |
| YSTRGDHTVL | 879 |
| TNARGDLTVS | 880 |
| TNARGDYTAL | 881 |
| AEGRGDTRGS | 882 |
| TNIRGDQSSV | 883 |
| VDRRGDTMTM | 884 |
| QIGRGDVTPL | 885 |
| MQARGDLLGV | 886 |
| NTQRGDYASV | 887 |
| SDTRGDRITQ | 888 |
| SAIRGDYRDV | 889 |
| VGVRGDVHPL | 890 |
| TVLRGDAERL | 891 |
| ASTRGDLSGQ | 892 |
| DSRRGDGQNL | 893 |
| IDKRGDIQGL | 894 |
| TVTRGDLLVN | 895 |
| DSTRGDRLGL | 896 |
| AANRGDYASL | 897 |
| RDYRGDTSSL | 898 |
| SNIRGDASLH | 899 |
| NLQRGDRTDF | 900 |
| NASRGDHSSM | 901 |
| TSMRGDYTHV | 902 |
| TSRRGDLNDV | 903 |
| QSHRGDVQSL | 904 |
| STTRGDYQSV | 905 |
| YSTRGDHAML | 906 |
| TSARGDLIHT | 907 |
| ATARGDRESF | 908 |
| ASTRGDYSTM | 909 |
| SNNRGDVQRL | 910 |
| ERLRGDQHII | 911 |
| SSIRGDHATL | 912 |
| NPTRGDIATV | 913 |
| MSTRGDLSVN | 914 |
| MDRRGDITYA | 915 |
| NATRGDLMSA | 916 |
| SDHRGDRTYQ | 917 |
| NWVRGDLSTA | 918 |
| SSHRGDLSAT | 919 |
| NATRGDYREI | 920 |
| DTLRGDLQTA | 921 |
| IAVRGDLTPV | 922 |
| TLLRGDLAHS | 923 |
| MNTRGDHTVL | 924 |
| TGHRGDIAVY | 925 |
| TSNRGDQMSN | 926 |
| SATRGDHTAI | 927 |
| SSVRGDREHA | 928 |
| TNSRGDLVVH | 929 |
| ETRRGDLSSQ | 930 |
| TYARGDITLT | 931 |
| SASRGDTLHL | 932 |
| DNQRGDLSSR | 933 |
| TQSRGDLRDA | 934 |
| ENRRGDLTNA | 935 |
| TSIRGDISSV | 936 |
| ISARGDTMTL | 937 |
| SRVRGDQDHW | 938 |
| SGNRGDIASY | 939 |
| NEQRGDRYNH | 940 |
| EALRGDVRAL | 941 |
| VDRRGDYTQM | 942 |
| NNVRGDYTSV | 943 |
| QTYRGDASVL | 944 |
| SNYRGDHGSL | 945 |
| TSSRGDLREV | 946 |
| QYTRGDVSNL | 947 |
| SDLRGDVRER | 948 |
| QNARGDTTAL | 949 |
| NSLRGDRDTM | 950 |
| SNSRGDQAQV | 951 |
| EATRGDRSTL | 952 |
| VPQRGDITNL | 953 |
| DKIRGDLRSI | 954 |
| SAARGDYSIV | 955 |
| VGLRGDLIGQ | 956 |
| QAGRGDLTVI | 957 |
| SKTRGDFQEA | 958 |
| GTARGDYSML | 959 |
| ISTRGDLVTI | 960 |
| TGTRGDREYM | 961 |
| MTTRGDLSIT | 962 |
| STSRGDMTSV | 963 |
| GPARGDFTSS | 964 |
| VDRRGDLGHV | 965 |
| NSGRGDMSQL | 966 |
| SQGRGDLERY | 967 |
| VFGRGDQYTL | 968 |
| IVGRGDLHLQ | 969 |
| SYSRGDTSRL | 970 |
| MSSRGDRATI | 971 |
| MSSRGDLTRV | 972 |
| QHTRGDFGNL | 973 |
| ETRRGDLSSQ | 974 |
| TGTRGDLRAI | 975 |
| QMARGDLSTA | 976 |
| LVERGDRIMN | 977 |
| GLVRGDLNAR | 978 |
| NNPRGDLSSV | 979 |
| HEKRGDLSNS | 980 |
| SQARGDLHQT | 981 |
| FTGRGDLRTA | 982 |
| QSIRGDTGHL | 983 |
| SAGRGDMSVL | 984 |
| SERRGDEHKW | 985 |
| TTGRGDLVSL | 986 |
| LGLRGDQIGQ | 987 |
| VALRGDLTHQ | 988 |
| MIGRGDQAPL | 989 |
| FQARGDHSTL | 990 |
| NSVRGDHSSL | 991 |
| WPNRGDLQST | 992 |
| SASRGDYQTL | 993 |
| TMQRGDLHTI | 994 |
| TALRGDLAGT | 995 |
| VNARGDFSQT | 996 |
| GWIRGDRTSM | 997 |
| TFSRGDHTVL | 998 |
| SGARGDLVGV | 999 |
| NNVRGDYTSV | 1000 |
| QTARGDHVVL | 1001 |
| TLIRGDLTRS | 1002 |
| VTTRGDRLEL | 1003 |

TABLE 4
Top Ranking Skeletal Muscle
Specific n-mer inserts and/or RGD Motifs

| Variant | SEQ ID NO.: |
|---|---|
| EASRGDLQMR | 1004 |
| TYARGDLSGH | 1005 |
| DSSRGDRSSV | 1006 |
| SQSRGDLVPL | 1007 |
| MPARGDLVQT | 1008 |
| SGVRGDRLAV | 1009 |
| SNERGDQHRL | 1010 |
| STLRGDYAVS | 1011 |
| SNARGDIMTT | 1012 |
| TTGRGDQARL | 1013 |
| TTLRGDHSQL | 1014 |
| TNLRGDYQLT | 1015 |
| STSRGDYVTH | 1016 |
| SATRGDYSIL | 1017 |
| QATRGDLQHT | 1018 |
| HDDRGDRRML | 1019 |
| SSHRGDLLAT | 1020 |
| SYQRGDQHNM | 1021 |
| SHQRGDYNTL | 1022 |
| MDQRGDMSVV | 1023 |
| QIARGDLHVP | 1024 |
| TYTRGDQHMM | 1025 |
| DASRGDRTSL | 1026 |
| SDRRGDLHPQ | 1027 |
| SATRGDHSTL | 1028 |
| AYQRGDFTSV | 1029 |
| TGNRGDLQSV | 1030 |
| AWRRGDLENA | 1031 |
| NAARGDHGLL | 1032 |
| SYSRGDHQAL | 1033 |
| DGVRGDRTVV | 1034 |
| SSERGDKMST | 1035 |
| TSMRGDYTHV | 1036 |
| SGVRGDRIII | 1037 |
| ENRRGDAQLL | 1038 |
| TVYRGDREQH | 1039 |
| LDRRGDLLNS | 1040 |
| ESVRGDLRST | 1041 |
| DKYRGDQRGL | 1042 |
| TDRRGDAHIL | 1043 |
| SQTRGDTSLM | 1044 |
| DATRGDTRML | 1045 |
| SMQRGDLTHA | 1046 |
| SGTRGDTLIL | 1047 |
| ANTRGDHQNF | 1048 |
| TSQRGDHTQL | 1049 |
| SHWRGDTSSL | 1050 |
| ISGRGDRQEL | 1051 |
| SVLRGDIGTI | 1052 |
| QDSRGDLSGK | 1053 |
| KPIRGDGAEL | 1054 |
| TYMRGDLQHT | 1055 |
| TELRGDLSST | 1056 |
| SAMRGDHAVL | 1057 |
| SWVRGDASTL | 1058 |
| QYARGDPHAL | 1059 |
| STNRGDIVVH | 1060 |
| VNIRGDLHQT | 1061 |
| REYRGDSSIL | 1062 |
| STLRGDGSMM | 1063 |
| SSQRGDITAH | 1064 |
| TEFRGDRSTV | 1065 |
| TSTRGDYVTV | 1066 |
| NHLRGDVSAI | 1067 |
| LERRGDLVST | 1068 |
| NEYRGDHSHP | 1069 |
| NWQRGDTSTL | 1070 |
| TDSRGDRTVL | 1071 |
| YAVRGDQHTI | 1072 |
| FGVRGDTRDL | 1073 |
| SSARGDTGML | 1074 |
| SAMRGDQMGQ | 1075 |
| SHTRGDLVTT | 1076 |
| VTTRGDTAIL | 1077 |
| EPTRGDRHSI | 1078 |
| GSQRGDRLVL | 1079 |
| QHYRGDLSTT | 1080 |
| METRGDRQQL | 1081 |
| EALRGDKVVL | 1082 |
| NAVRGDQSYV | 1083 |
| SPLRGDNRAL | 1084 |
| ENRRGDTHAL | 1085 |
| NSQRGDLTGT | 1086 |
| DNSRGDHSRL | 1087 |
| NTHRGDYASI | 1088 |
| HTTRGDQQPL | 1089 |
| EWRRGDQTAL | 1090 |
| TAVRGDQTTV | 1091 |
| EYLRGDRISQ | 1092 |
| SRYRGDQETI | 1093 |
| ASLRGDTVTL | 1094 |
| VGVRGDSHVL | 1095 |
| TNSRGDHTSL | 1096 |
| VDQRGDQRAL | 1097 |
| ASVRGDRLDV | 1098 |
| TSYRGDLSSN | 1099 |
| TSDRGDSSNR | 1100 |
| SYMRGDLDMR | 1101 |
| TDVRGDRSMI | 1102 |
| SYVRGDSSTV | 1103 |
| QVYRGDRESV | 1104 |
| TDRRGDAVSF | 1105 |
| TNSRGDLHTT | 1106 |
| EAIRGDFKTL | 1107 |
| TGIRGDTAGL | 1108 |
| TSSRGDVQSL | 1109 |
| SSRRGDTNEL | 1110 |
| SNARGDHTPL | 1111 |
| SELRGDRSPY | 1112 |
| VHARGDTSIM | 1113 |
| SALRGDHGTL | 1114 |
| QTGRGDIAGM | 1115 |
| TVARGDQIRL | 1116 |
| TNSRGDSVAL | 1117 |
| QNARGDSAVI | 1118 |
| HYLRGDTTTL | 1119 |
| ANARGDLQNT | 1120 |
| QNIRGDIRTV | 1121 |
| AALRGDHGQM | 1122 |
| TTDRGDLSAR | 1123 |
| LNVRGDLVHT | 1124 |
| YSTRGDHTVL | 1125 |
| TNARGDLTVS | 1126 |
| TNARGDYTAL | 1127 |
| AEGRGDTRGS | 1128 |
| TNIRGDQSSV | 1129 |
| VDRRGDTMTM | 1130 |
| QIGRGDVTPL | 1131 |
| MQARGDLLGV | 1132 |
| NTQRGDYASV | 1133 |
| SDTRGDRITQ | 1134 |
| SAIRGDYRDV | 1135 |
| VGVRGDVHPL | 1136 |
| TVLRGDAERL | 1137 |
| ASTRGDLSGQ | 1138 |
| DSRRGDGQNL | 1139 |
| IDKRGDIQGL | 1140 |
| TVTRGDLLVN | 1141 |
| DSTRGDRLGL | 1142 |
| AANRGDYASL | 1143 |
| RDYRGDTSSL | 1144 |
| SNIRGDASLH | 1145 |
| NLQRGDRTDF | 1146 |
| NASRGDHSSM | 1147 |
| TSMRGDYTHV | 1148 |
| TSRRGDLNDV | 1149 |
| QSHRGDVQSL | 1150 |
| STTRGDYQSV | 1151 |
| YSTRGDHAML | 1152 |
| TSARGDLIHT | 1153 |
| ATARGDRESF | 1154 |
| ASTRGDYSTM | 1155 |
| SNNRGDVQRL | 1156 |
| ERLRGDQHII | 1157 |
| SSIRGDHATL | 1158 |
| NPTRGDIATV | 1159 |
| MSTRGDLSVN | 1160 |
| MDRRGDITYA | 1161 |
| NATRGDLMSA | 1162 |
| SDHRGDRTYQ | 1163 |
| NWVRGDLSTA | 1164 |
| SSHRGDLSAT | 1165 |
| NATRGDYREI | 1166 |
| DTLRGDLQTA | 1167 |
| IAVRGDLTPV | 1168 |
| TLLRGDLAHS | 1169 |
| MNTRGDHTVL | 1170 |
| TGHRGDIAVY | 1171 |
| TSNRGDQMSN | 1172 |
| SATRGDHTAI | 1173 |
| SSVRGDREHA | 1174 |
| TNSRGDLVVH | 1175 |
| ETRRGDLSSQ | 1176 |
| TYARGDITLT | 1177 |
| SASRGDTLHL | 1178 |
| DNQRGDLSSR | 1179 |
| TQSRGDLRDA | 1180 |
| ENRRGDLTNA | 1181 |
| TSIRGDISSV | 1182 |
| ISARGDTMTL | 1183 |
| SRVRGDQDHW | 1184 |
| SGNRGDIASY | 1185 |
| NEQRGDRYNH | 1186 |
| EALRGDVRAL | 1187 |
| VDRRGDYTQM | 1188 |
| NNVRGDYTSV | 1189 |
| QTYRGDASVL | 1190 |
| SNYRGDHGSL | 1191 |
| TSSRGDLREV | 1192 |
| QYTRGDVSNL | 1193 |
| SDLRGDVRER | 1194 |
| QNARGDTTAL | 1195 |
| NSLRGDRDTM | 1196 |
| SNSRGDQAQV | 1197 |
| EATRGDRSTL | 1198 |
| VPQRGDITNL | 1199 |
| DKIRGDLRSI | 1200 |
| SAARGDYSIV | 1201 |
| VGLRGDLIGQ | 1202 |
| QAGRGDLTVI | 1203 |
| SKTRGDFQEA | 1204 |
| GTARGDYSML | 1205 |
| ISTRGDLVTI | 1206 |
| TGTRGDREYM | 1207 |
| MTTRGDLSIT | 1208 |
| STSRGDMTSV | 1209 |
| GPARGDFTSS | 1210 |
| VDRRGDLGHV | 1211 |
| NSGRGDMSQL | 1212 |
| SQGRGDLERY | 1213 |
| VFGRGDQYTL | 1214 |
| IVGRGDLHLQ | 1215 |
| SYSRGDTSRL | 1216 |
| MSSRGDRATI | 1217 |
| MSSRGDLTRV | 1218 |
| QHTRGDFGNL | 1219 |
| ETRRGDLSSQ | 1220 |
| TGTRGDLRAI | 1221 |
| QMARGDLSTA | 1222 |
| LVERGDRIMN | 1223 |
| GLVRGDLNAR | 1224 |
| NNPRGDLSSV | 1225 |
| HEKRGDLSNS | 1226 |
| SQARGDLHQT | 1227 |
| FTGRGDLRTA | 1228 |
| QSIRGDTGHL | 1229 |
| SAGRGDMSVL | 1230 |
| SERRGDEHKW | 1231 |
| TTGRGDLVSL | 1232 |
| LGLRGDQIGQ | 1233 |
| VALRGDLTHQ | 1234 |
| MIGRGDQAPL | 1235 |
| FQARGDHSTL | 1236 |
| NSVRGDHSSL | 1237 |
| WPNRGDLQST | 1238 |
| SASRGDYQTL | 1239 |
| TMQRGDLHTI | 1240 |
| TALRGDLAGT | 1241 |
| VNARGDFSQT | 1242 |
| GWIRGDRTSM | 1243 |
| TFSRGDHTVL | 1244 |
| SGARGDLVGV | 1245 |
| NNVRGDYTSV | 1246 |
| QTARGDHVVL | 1247 |
| TLIRGDLTRS | 1248 |
| VTTRGDRLEL | 1249 |

TABLE 5
VP1 capsid proteins

| SEQ | |
|---|---|
| ID | |
| NO.: | VP1 Capsid Protein Sequence |
| 1250 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKTGQQPAKKRLNFGQTGDS |
| | ESVPDPQPIGEPPAGPSGLGSGTMAAGGGAPMADNNEGADGVGSSSGNWHCDSAWLGDRV |
| | ITTSTRPWALPTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSEYQLPYVLGSA |
| | HQGCPPPFPADVFMIPQYGYLTLNNGSQAVGRSSFYCLEYFPSQMRRTGNNFEFSYQFED |
| | VPFHSSYAHSQSLDRLMNPLIDQYLYYLSRTQSTGGTAGTQQLLFSQAGPNNMSAQAKNW |
| | LPGPCYRQQRVSTTLSQNNNSNFAWTGATKYHLNGRDSLVNPGVAMATHKDDEERFFPSS |
| | GVLMFGKQGAGKDNVDYSSVMLTSEEEIKTTNPVATEQYGVVADNLQQQNAAPIVGAVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1251 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKTGQQPAKKRLNFGQTGDS |
| | ESVPDPQPIGEPPAGPSGLGSGTMAAGGGAPMADNNEGADGVGSSSGNWHCDSAWLGDRV |
| | ITTSTRPWALPTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSEYQLPYVLGSA |
| | HQGCPPPFPADVFMIPQYGYLTLNNGSQAVGRSSFYCLEYFPSQMRRTGNNFEFSYQFED |
| | VPFHSSYAHSQSLDRLMNPLIDQYLYYLSRTQSTGGTAGTQQLLFSQAGPNNMSAQAKNW |
| | LPGPCYRQQRVSTTLSQNNNSNFAWTGATKYHLNGRDSLVNPGVAMATHKDDEERFFPSS |
| | GVLMFGKQGAGKDNVDYSSVMLTSEEEIKTTNPVATEQYGVVADNLQQQNAAPIVGAVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1252 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQSNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGLGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1253 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQRPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKPFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLHYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1254 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPAKKRLSFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNSEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1255 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1256 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1257 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1258 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGPVEEAAKTAPGKKRPVEPPPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTVANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1259 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQDEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSARAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQANTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNSE |
| | GTYSEPRPIGTRYLTRNL |
| 1260 | MAADGYLPDWLEDNLSEGIREWWALKPGAPQPKANQQHQDNARGLVLPGYKYLGPGNGLD |
| | KGEPVNAADAAALEHDKAYDQQLKAGDNPYLKYNHADAEFQERLKEDTSFGGNLGRAVFQ |
| | AKKRLLEPLGLVEEAAKTAPGKKRPVEQSPQEPDSSAGIGKSGAQPAKKRLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWPGASSWALNGRNSLMNPGPAMASHKEGEDRFFPLSGS |
| | LIFGKQGTGRDNVDADKVMITNEEEIKTTNPVATESYGQVATNHQSNSRGDYNSLAQAQT |
| | GWVQNQGILPGMVWQDRDVYLQGPIWAKIPHTDGNFHPSPLMGGFGMKHPPPQILIKNTP |
| | VPADPPTAFNKDKLNSFITQYSTGQVSVEIEWELQKENSKRWNPEIQYTSNYYKSNNVEF |
| | AVNTEGVYSEPRPIGTRYLTRNL |
| 1261 | MAADGYLPDWLEDNLSEGIREWWALKPGAPQPKANQQHQDNARGLVLPGYKYLGPGNGLD |
| | KGEPVNAADAAALEHDKAYDQQLKAGDNPYLKYNHADAEFQERLKEDTSFGGNLGRAVFQ |
| | AKKRLLEPLGLVEEAAKTAPGKKRPVEQSPQEPDSSAGIGKSGAQPAKKRLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWAAASSWALNGRNSLMNPGPAMASHKEGEDRFFPLSGS |
| | LIFGKQGTGRDNVDADKVMITNEEEIKTTNPVATESYGQVATNHQGQSGRGDLGLSAQAA |
| | QTGWVQNQGILPGMVWQDRDVYLQGPIWAKIPHTDGNFHPSPLMGGFGMKHPPPQILIKN |
| | TPVPADPPTAFNKDKLNSFITQYSTGQVSVEIEWELQKENSKRWNPEIQYTSNYYKSNNV |
| | EFAVNTEGVYSEPRPIGTRYLTRNL |
| 1262 | MAADGYLPDWLEDNLSEGIREWWALKPGAPQPKANQQHQDNARGLVLPGYKYLGPGNGLD |
| | KGEPVNAADAAALEHDKAYDQQLKAGDNPYLKYNHADAEFQERLKEDTSFGGNLGRAVFQ |
| | AKKRLLEPLGLVEEAAKTAPGKKRPVEQSPQEPDSSAGIGKSGAQPAKKRLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWPGASSWALNGRNSLMNPGPAMASHKEGEDRFFPLSGS |
| | LIFGKQGTGRDNVDADKVMITNEEEIKTTNPVATESYGQVATNHQSGQSGRGDLGLSAQA |
| | ATGWVQNQGILPGMVWQDRDVYLQGPIWAKIPHTDGNFHPSPLMGGFGMKHPPPQILIKN |
| | TPVPADPPTAFNKDKLNSFITQYSTGQVSVEIEWELQKENSKRWNPEIQYTSNYYKSNNV |
| | EFAVNTEGVYSEPRPIGTRYLTRNL |
| 1263 | MAADGYLPDWLEDNLSEGIREWWALKPGAPQPKANQQHQDNARGLVLPGYKYLGPGNGLD |
| | KGEPVNAADAAALEHDKAYDQQLKAGDNPYLKYNHADAEFQERLKEDTSFGGNLGRAVFQ |
| | AKKRLLEPLGLVEEAAKTAPGKKRPVEQSPQEPDSSAGIGKSGAQPAKKRLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWPGASSWALNGRNSLMNPGPAMASHKEGEDRFFPLSGS |
| | LIFGKQGTGRDNVDADKVMITNEEEIKTTNPVATESYGQVATNHQGQSGRGDAVGVAQAA |
| | QTGWVQNQGILPGMVWQDRDVYLQGPIWAKIPHTDGNFHPSPLMGGFGMKHPPPQILIKN |
| | TPVPADPPTAFNKDKLNSFITQYSTGQVSVEIEWELQKENSKRWNPEIQYTSNYYKSNNV |
| | EFAVNTEGVYSEPRPIGTRYLTRNL |
| 1264 | MAADGYLPDWLEDNLSEGIREWWALKPGAPQPKANQQHQDNARGLVLPGYKYLGPGNGLD |
| | KGEPVNAADAAALEHDKAYDQQLKAGDNPYLKYNHADAEFQERLKEDTSFGGNLGRAVFQ |
| | AKKRLLEPLGLVEEAAKTAPGKKRPVEQSPQEPDSSAGIGKSGAQPAKKRLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWPGASSWALNGRNSLMNPGPAMASHKEGEDRFFPLSGS |
| | LIFGKQGTGRDNVDADKVMITNEEEIKTTNPVATESYGQVATNHQSGQSGRGDAVGVAQA |
| | ATGWVQNQGILPGMVWQDRDVYLQGPIWAKIPHTDGNFHPSPLMGGFGMKHPPPQILIKN |
| | TPVPADPPTAFNKDKLNSFITQYSTGQVSVEIEWELQKENSKRWNPEIQYTSNYYKSNNV |
| | EFAVNTEGVYSEPRPIGTRYLTRNL |
| 1265 | MAADGYLPDWLEDNLSEGIREWWALKPGAPQPKANQQHQDNARGLVLPGYKYLGPGNGLD |
| | KGEPVNAADAAALEHDKAYDQQLKAGDNPYLKYNHADAEFQERLKEDTSFGGNLGRAVFQ |
| | AKKRLLEPLGLVEEAAKTAPGKKRPVEQSPQEPDSSAGIGKSGAQPAKKRLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWAAASSWALNGRNSLMNPGPAMASHKEGEDRFFPLSGS |
| | LIFGKQGTGRDNVDADKVMITNEEEIKTTNPVATESYGQVATNHQSGQSGRGDLGLSAQA |
| | ATGWVQNQGILPGMVWQDRDVYLQGPIWAKIPHTDGNFHPSPLMGGFGMKHPPPQILIKN |
| | TPVPADPPTAFNKDKLNSFITQYSTGQVSVEIEWELQKENSKRWNPEIQYTSNYYKSNNV |
| | EFAVNTEGVYSEPRPIGTRYLTRNL |
| 1266 | MAADGYLPDWLEDNLSEGIREWWALKPGAPQPKANQQHQDNARGLVLPGYKYLGPGNGLD |
| | KGEPVNAADAAALEHDKAYDQQLKAGDNPYLKYNHADAEFQERLKEDTSFGGNLGRAVFQ |
| | AKKRLLEPLGLVEEAAKTAPGKKRPVEQSPQEPDSSAGIGKSGAQPAKKRLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWAAASSWALNGRNSLMNPGPAMASHKEGEDRFFPLSGS |
| | LIFGKQGTGRDNVDADKVMITNEEEIKTTNPVATESYGQVATNHQGQSGRGDAVGVAQAA |
| | QTGWVQNQGILPGMVWQDRDVYLQGPIWAKIPHTDGNFHPSPLMGGFGMKHPPPQILIKN |
| | TPVPADPPTAFNKDKLNSFITQYSTGQVSVEIEWELQKENSKRWNPEIQYTSNYYKSNNV |
| | EFAVNTEGVYSEPRPIGTRYLTRNL |
| 1267 | MAADGYLPDWLEDNLSEGIREWWALKPGAPQPKANQQHQDNARGLVLPGYKYLGPGNGLD |
| | KGEPVNAADAAALEHDKAYDQQLKAGDNPYLKYNHADAEFQERLKEDTSFGGNLGRAVFQ |
| | AKKRLLEPLGLVEEAAKTAPGKKRPVEQSPQEPDSSAGIGKSGAQPAKKRLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWAAASSWALNGRNSLMNPGPAMASHKEGEDRFFPLSGS |
| | LIFGKQGTGRDNVDADKVMITNEEEIKTTNPVATESYGQVATNHQSGQSGRGDAVGVAQA |
| | ATGWVQNQGILPGMVWQDRDVYLQGPIWAKIPHTDGNFHPSPLMGGFGMKHPPPQILIKN |
| | TPVPADPPTAFNKDKLNSFITQYSTGQVSVEIEWELQKENSKRWNPEIQYTSNYYKSNNV |
| | EFAVNTEGVYSEPRPIGTRYLTRNL |
| 1268 | MAADGYLPDWLEDNLSEGIREWWALKPGAPKPKANQQKQDDGRGLVLPGYKYLGPFNGLD |
| | KGEPVNAADAAALEHDKAYDQQLQAGDNPYLRYNHADAEFQERLQEDTSFGGNLGRAVFQ |
| | AKKRLLEPLGLVEEAAKTAPGKKRPVEQSPQEPDSSAGIGKSGAQPAKKRLNFGQTGDTE |
| | SVPDPQPIGEPPAAPSGVGSLTMASGGGAPVADNNEGADGVGNASGNWHCDSQWLGDRVI |
| | TTSTRTWALPTYNNHLYKQISSASTGATNDNTYFGYSTPWGYFDFNRFHCHFSPRDWQRL |
| | INNNWGFRPKRLNFKLFNIQVKEVTDNNGVKTIANNLTSTVQVFTDSDYQLPYVLGSAHE |
| | GCLPPFPADVFMIPQYGYLTLNDGSQAVGRSSFYCLEYFPSQMLRTGNNFQFSYEFENVP |
| | FHSSYAHSQSLDRLMNPLIDQYLYYLSKTINGSGQNQQTLKFSVAGPSNMAVQGRNYIPG |
| | PSYRQQRVSTTVTQNNNSEFAWPGASSWALNGRNSLMNPGPAMASHKEGEDRFFPLSGSL |
| | IFGKQGTGRDNVDADKVMITNEEEIKTTNPVATESYGQVATNHQGQSGRGDLGLSAQAAQ |
| | TGWVQNQGILPGMVWQDRDVYLQGPIWAKIPHTDGNFHPSPLMGGFGMKHPPPQILIKNT |
| | PVPADPPTAFNKDKLNSFITQYSTGQVSVEIEWELQKENSKRWNPEIQYTSNYYKSNNVE |
| | FAVNTEGVYSEPRPIGTRYLTRNL |
| 1269 | MAADGYLPDWLEDNLSEGIREWWALKPGAPQPKANQQHQDNARGLVLPGYKYLGPGNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKTGQQPAKKRLNFGQTGDT |
| | ESVPDPQPIGEPPAAPAAVGPTTMASGGGAPMADNNEGADGVGNASGNWHCDSTWLGDRV |
| | ITTSTRTWALPTYNNHLYKQISNGTSGGATNDNTYFGYSTPWGYFDFNRFHCHFSPRDWQ |
| | RLINNNWGFRPKRLNFKLFNIQVKEVTDNNGVKTIANNLTSTVQVFTDSDYQLPYVLGSA |
| | HEGCLPPFPADVFMIPQYGYLTLNDGSQAVGRSSFYCLEYFPSQMLRTGNNFQFSYEFEN |
| | VPFHSSYAHSQSLDRLMNPLIDQYLYYLSKTINGSGQNQQTLKFSVAGPSNMAVQGRNYI |
| | PGPSYRQQRVSTTVTQNNNSEFAWPGASSWALNGRNSLMNPGPAMASHKEGEDRFFPLSG |
| | SLIFGKQGTGRDNVDADKVMITNEEEIKTTNPVATESYGQVATNHQGQSGRGDLGLSAQA |
| | AQTGWVQNQGILPGMVWQDRDVYLQGPIWAKIPHTDGNFHPSPLMGGFGMKHPPPQILIK |
| | NTPVPADPPTAFNKDKLNSFITQYSTGQVSVEIEWELQKENSKRWNPEIQYTSNYYKSNN |
| | VEFAVNTEGVYSEPRPIGTRYLTRNL |
| 1270 | MAADGYLPDWLEDNLSEGIREWWALKPGAPQPKANQQHQDNARGLVLPGYKYLGPGNGLD |
| | KGEPVNAADAAALEHDKAYDQQLKAGDNPYLKYNHADAEFQERLKEDTSFGGNLGRAVFQ |
| | TKKRVLEPFGLVEEGAKTAPGKKRPVEQSPQEPDSSSGIVKTGQQPAKKRLNFGQTGDTE |
| | SVPDPQPLGEPPATPSGVGSLTMASGGGAPMADNNEGADGVGNASGNWHCDSTWLGDRVI |
| | TTSTRTWALPTYNNHLYKQISNGTSGGATNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWPGASSWALNGRNSLMNPGPAMASHKEGEDRFFPLSGS |
| | LIFGKQGTGRDNVDADKVMITNEEEIKTTNPVATESYGQVATNHQGQSGRGDLGLSAQAA |
| | QTGWVQNQGILPGMVWQDRDVYLQGPIWAKIPHTDGNFHPSPLMGGFGMKHPPPQILIKN |
| | TPVPADPPTAFNKDKLNSFITQYSTGQVSVEIEWELQKENSKRWNPEIQYTSNYYKSNNV |
| | EFAVNTEGVYSEPRPIGTRYLTRNL |
| 1271 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPFGLVEEGAKTAPGKKRPVEQSPQEPDSSSGIGKTGQQPAKKRLNFGQTGDSE |
| | SVPDPQPLGEPPATPAAVGPTTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWPGASSWALNGRNSLMNPGPAMASHKEGEDRFFPLSGS |
| | LIFGKQGTGRDNVDADKVMITNEEEIKTTNPVATESYGQVATNHQGQSGRGDLGLSAQAA |
| | QTGWVQNQGILPGMVWQDRDVYLQGPIWAKIPHTDGNFHPSPLMGGFGLKHPPPQILIKN |
| | TPVPADPPAEFSATKFASFITQYSTGQVSVEIEWELQKENSKRWNPEVQYTSNYAKSANV |
| | DFTVDNNGLYTEPRPIGTRYLTRPL |
| 1272 | MAADGYLPDWLEDNLSEGIREWWALKPGAPQPKANQQKQDDGRGLVLPGYKYLGPFNGLD |
| | KGEPVNEADAAALEHDKAYDQQLKAGDNPYLRYNHADAEFQERLQEDTSFGGNLGRAVFQ |
| | AKKRVLEPLGLVEEGAKTAPGKKRPVEPDSSSGIGKSGQQPAKKRLNFGQTGDSESVPDP |
| | QPLSEPPAGPSGLGSGTMAAGGGAPMADNNEGADGVGNVSGNWHCDSTWLGDRVITTSTR |
| | TWALPTYNNHLYKQISNGTSGGSSNDNTYFGYSTPWGYFDFNRFHCHFSPRDWQRLINNN |
| | WGFRPKKLNFKLFNIQVKEVTQNEGTKTIANNLTSTVQVFTDSEYQLPYVLGSAHQGCLP |
| | PFPADVFMIPQYGYLTLNNGSQAVGRSSFYCLEYFPSQMLRTGNNFTFSYTFEDVPFHSS |
| | YAHSQSLDRLMNPLIDQYLYYLSRTQTNGTNATQTLLFAQAGPQNMSAQAKNWLPGPCYR |
| | QQRVSTTVSQNNNSNFTWTGATKYHLNGRDSLVSPGVAMATHKDDEERFFPSSGVLMFGK |
| | QGAGKDNVEYTNVMLTSEEEIKTTNPVATEQYGVVADNLQQTNSAPIVGAVNSQGALPGM |
| | VWQNRDVYLQGPIWAKIPHTDGNFHPSPLMGGFGLKHPPPQILIKNTPVPADPPVNFTDA |
| | KLASFITQYSTGQVSVEIEWELQKENSKRWNPEIQYTSNYYKSANVDFAVNADGVYSEPR |
| | PIGTRYLTRNL |
| 1273 | MAADGYLPDWLEDNLSEGIREWWALKPGAPQPKANQQKQDDGRGLVLPGYKYLGPFNGLD |
| | KGEPVNEADAAALEHDKAYDQQLKAGDNPYLRYNHADAEFQERLQEDTSFGGNLGRAVFQ |
| | AKKRVLEPLGLVEEGAKTAPGKKRPVEPDSSSGIGKSGQQPAKKRLNFGPTGDSESVPDP |
| | QPLSEPPAGPSGLGSGTMAAGGGAPMADNNEGADGVGNVSGNWHCDSTWLGDRVITTSTR |
| | TWALPTYNNHLYKQISNGTSGGSSNDNTYFGYSTPWGYFDFNRFHCHFSPRDWQRLINNN |
| | WGFRPKKLNFKLFNIQVKEVTQNEGTKTTANNLTSTVQVFTDSEYQLPYVLGSAHQGCLP |
| | PFPADVFMIPQYGYLTLNNGSQAVGRSSFYCLEYFPSQMLRTGNNFTFSYTFEDVPFHSS |
| | YAHSQSLDRLMNPLIDQYLYYLSRTQTNGTNATQTLLFAQAGPQNMSAQAKNWLPGPCYR |
| | QQRVSTAVSQNNNSNFTWTGATKYHLNGRDSLVNPGVAMATHKDDEERFFPSSGVLMFGK |
| | QGAGKDNVEYTNVMLTSEEEIKTTNPVATEQYGVVADNLQQTNSAPIVGAVNSQGALPGM |
| | VWQNRDVYLQGPIWAKIPHTDGNFHPSPLMGGFGLKHPPPQILIKNTPVPADPPVNFTDA |
| | KLASFITQYSTGQVSVEIEWELQKENSKRWNPEIQYTSNYYKSANVDFAVNADGVYSEPR |
| | PIGTRYLTRNL |
| 1274 | MAADGYLPDWLEDNLSEGIREWWALKPGAPQPKANQQKQDDGRGLVLPGYKYLGPFNGLD |
| | KGEPVNEADAAALEHDKAYDQQLKAGDNPYLRYNHADAEFQERLQEDTSFGGNLGRAVFQ |
| | AKKRVLEPLGLVEEGAKTAPGKKRPVEPDSSSGIGKSGRQPAKKRLNFGQTGDSESVPDP |
| | QPLSEPPAGPSGLGSGTMAAGGGAPMADNNEGADGVGNASGNWHCDSTWLGDRVITTSTR |
| | TWALPTYNNHLYKQISNGTSGGSSNDNTYFGYSTPWGYFDFNRFHCHFSPRDWQRLINNN |
| | WGFRPKRLNFKLFNIQVKEVTQNEGTKTIANNLTSTVQVFTDSKYQLPYVLGSAHQGCLP |
| | PFPADVFMIPQYGYLTLNNGSQAVGRSSFYCLEYFPSQMLRTGNNFTFSYTFEDVPFHSS |
| | YAHSQSLDRLMNPLIDQYLYYLSRTQTNGTNATQTLLFAQAGPQNMSAQAKNWLPGPCYR |
| | QQRVSTTVSQNNNSNFTWTGATKYHLNGRDSLVNPGVAMATHKDDEERFFPSSGVLMFGK |
| | QGAGKDNVEYTNVMLTSEEEIKTTNPVATEQYGVVADNLQQTNSAPIVGAVNSQGALPGM |
| | VWQNRDVYLQGPIWAKIPHTDGNFHPSPLMGGFGLKHPPPQILIKNTPVPADPPVNFTDA |
| | KLASFITQYSTGQVSVEIEWELQKENSKRWNPEIQYTSNYYKSANVDFAVNADGVYSEPR |
| | PIGTRYLTRNL |
| 1275 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANSLTSTIQVFTDSEYQLPYVLGSA |
| | HQGCLPPFPADVFMIPQYGYLTPNNGSQAVGRSSFYCLEYFPSQMLRTGNNFSFSYTFED |
| | VPFHSSYAHSQSLDRLMNPLIDQYLYYLSRTQSTGGTAGTQQLLSSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWELQKENSKRWNPEIQYTSNYYKSTNVDFAVNTE |
| | GVYSEPRPIGTRYLTRNL |
| 1276 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYVHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNSGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWELQKENSKRWNPEIQYTSNYYKSTNVDFAVNTE |
| | GVYSEPRPIGTRYPTRNL |
| 1277 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPAYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLSGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSSITQYSTGQVSVEIEWELQKENSKRWNPEIQYTSNYYKSTNVDFAVNTE |
| | GVYSEPRPIGTRYLTRNL |
| 1278 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIVWELQKENSKRRNPEIQYTSNYYKSTNVDFAVNTE |
| | GVYSEPRPIGTRYLTRNL |
| 1279 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPPS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVVIEWELQKENSKRWNPEIQYTSNYYKSTNVDFAVNTE |
| | GVYSELRPIGTRYLTRNL |
| 1280 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKATNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWELQKENSKRWNPEIQYTSNYYKSTNVDFAVNTE |
| | GVYSEPRPIGTRYLTRNL |
| 1281 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGRTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYLDFNRFHCHFSPRDWQ |
| | RLINNNWGFRPKRLSFKLFNIQVKEVTQNEGTKTIANNLASTIQVFTDSEYQPPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSRAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPTVGAVNS |
| | QGALPGMVWQNRDVYLQGPIWAKIPHTDGNFHPSPLMGGFGLKHPPPQILIKNTPVPADP |
| | PTAFNQAKLNSFIAQYSTGQVSVEIEWELQKENSKRWNPEIQYTSNYYKSTNADFAVNTE |
| | GVYSEPRPIGTRYLTRNL |
| 1282 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPHLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQLIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGNLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNMGYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYGTGQVSVEIEWELQKENSKRWNPEIQYTSNYYKSTNVDFAVNTE |
| | GVYSEPRPIGTRYLTRNL |
| 1283 | MVADGYLPDWLEDNLSEGIREWWDLKPGAPKPKANQQKQGDGRGLVLPGYKYLGPFNGLD |
| | KGEPVNAADAAALEHDKAYDQQLKAGDNPYLRYNHADAELQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCQPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWEPQKENSKRWNPEIQYTSNYYKSTNVDFAVNTE |
| | GVYSEPRPIGTRYLTRNL |
| 1284 | MAADGYLPDWLEDNLSEGIREWWALKPGAPKPKANQQKQDDGRGLVLPGYKYLGPFNGLD |
| | KGEPVNAADAAALEHDKAYDQQLQAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQTSNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSAEIEWELQKENSKRWNPEIQYTSNYYKSTNVDFAVNTE |
| | GVYSEPRPIGTRYLTRNL |
| 1285 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKSTPVPADP |
| | PTAFNQAKLNSFITQYSTGQVSVEIEWELQKENSKCWNPEIQYTSNYYKSTNVDFAVNTE |
| | GVYSEPRPIGTRYLTRNL |
| 1286 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RPINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYPFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQDTAPIVGAVNS |
| | QGALPGMVWQNRDVYLQGPIWAKIPHTDGNFHPSPLMGGFGLKHPPPQVLIKNTPVPADP |
| | PTAFNQAKLNSFITQYSTGQVSVEIEWELQKENSKRWNPEIQYTSNYYKSTNVDFAVNTE |
| | GVYSEPRPIGTRYLTRNL |
| 1287 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPAGKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLVDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWELQKENSKRWSPEIQYTSNYYKSTNVDFAVNTE |
| | GVYSEPRPIGTRYLTRNL |
| 1288 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQAVGRSSFYCLEYFPSQMLRTGNNFSFSYTFEDV |
| | PFHSSYAHSQSLDRLMNPLIDQYLYYLSRTQSTGGTAGTQQLLFSQAGPSNMSTQARNWL |
| | PGPCYRQQRVSTTLSQNNNSNFAWTGATKYHLNGRDSLVNPGVAMATNKDDEDRFFPSSG |
| | ILMFGKQGAGKDNVDYSNVMLTSEEEIKTTNPVATEQYGVVADNLQQQNTAPIVGAVNSQ |
| | GALPGMVWQNRDVYLQGPIWAKIPHTDGNFHPSPLMGGFGLKHPPPQILIKNTPVPADPP |
| | TAFNQAKLNSFITQYSTGQVSVEIEWELQKENSKRWNPEIQYTSNYYKSTNVDFAVNTEG |
| | AYSEPRPIGTRYLTRN |
| 1289 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNPTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWELQKENSKRWNPEIQYTSNYYKSTNVDFAVNTE |
| | GVYSEPRPIGTRYLTRNL |
| 1290 | MAADGYLPDWLEDNLSEGIREWWDLKPGAPKPKANQQKQDDGRGLVLPGYKYLGPFNGLD |
| | KGEPVSAADAAALEHDKACDQRLKAGDNPYLRYNHADAEFQERLQEDTSFGGNLGRAVFQ |
| | AKKRVLEPLGLVEEAAKTAPGKKGPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDEQTGVDFAVDSQG |
| | VYSEPRPIGTRYLTRNL |
| 1291 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPRPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1292 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVLQ |
| | AKKRVLEPLGLVEEAAKTAPGKKRPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1293 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLAEEAAKTAPGKKRPVEPSPQRSPDSSTGIGKKGQQPARKRLNFGQTGDS |
| | ESVPDPQPIGEPPAGPSGLGSGTMAAGGGAPMADNNKGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTGDGVTTIANNLTSTVQVFSDSEYQLPYVLGSAH |
| | QGCLPPFPADVFMIPQYGYLTLNNDSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1294 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPARKRLNFGQTGDS |
| | ESVPDPQPIGEPPAGPSGLGSGTMAAGGGAPMADNNKGADGVGNASGNWHCDSTWLGDRV |
| | ITTSTRTWALPTYNNHLYKQISSQSAGSTNDNVYFGYSTPWGYFDFNRFHCHFSPRDWQR |
| | LINSNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1295 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPARKRLNFGQTGDS |
| | ESVPDPQPLGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQVLRTGNNFTFSYTFEDV |
| | PFHSSYAHSQSLDRLMNPLIDQYLYYLARTQSNPGGTSGNRELQFYQGGPSTMAEQAKNW |
| | LPGPCFRQQRVSKTLDQNNNSNFAWTGATKYHLNGRNSLVNPGVAMATHKDDEDRFFPSS |
| | GVLIFGKTGATNKTTLENVLMTNEEEIRPTNPVATEEYGIVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1296 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTRLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCFRQRRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1297 | MAADGYLPDWLEDNLSEGIREWWDLKPGAPKPKANQQKQDDGRGLVLPGYKHLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTVQVFSDSEYQLPYVLGSA |
| | HQGCLPPFPADVFMIPQYGYLTLNNGSQSVGRSSFYCLEYFPSQMLRTGNNFTFSYTFED |
| | VPFHSSYAHSQSLDRLMNPLIDQYLYYLARTQSNAGGTAGNRELQFYQGGPTTMAEQAKN |
| | WLPGPCFRQQRVSKTLDQNNNSNFAWTGATKYHLNGRNSLVNPGVAMATHKDDEERFFPS |
| | SGVLIFGKTGAANKTTLENVLMTNEEEIRPTNPVATEEYGTVSSNLQAANTAAQTQVVNN |
| | QGALPGMVWQNRDVYLQGPIWAKIPHTDGNFHPSPLMGGFGLKHPPPQILIKNTPVPANP |
| | PEVFTPAKFASFITQYSTGQVSVEIEWELQKENSKRWNPEIQYTSNFDKQTGVDFAVDSQ |
| | GVYSEPRPIGTRYLTRNL |
| 1298 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTISYTFEDV |
| | PFHSSYAHSQSLDRLMNPLIDQYLYYLARTQSNAGGTAGNQELQFYQGGPTTMAEQAKNW |
| | LPGPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPADPP |
| | TAFNQAKLNSFITQYSTGQVSVEIEWELQKENSKRWNPEIQYTSNYYKSTNVDFAVNTEG |
| | VYSEPRPIGTRYLTRNL |
| 1299 | MAADGYLPDWLEDNLSEGIHEWWDPKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTHNNHLYKQISNGTSGGSTNDNVYFGYSTPWGYFDFNRFHCHFSPRDRQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTEGTAGTQQLLFSQAGPSNMSAQARNW |
| | LPGPCYRQQRVSTTLSQNNNSNFAWTGATKYHLNGRDSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPQILIKNTPVPANPPEV |
| | FTPAKFASFITQYSTGQVSVEIEWELQKENSKRWNPEIQYTSNFDKQTGVDFAVDSQGVY |
| | SEPRPIGTRYLTRNL |
| 1300 | MAADGYLPDWLEDTLSEGIRQWWKLKPGPPPPKPAERHKDDSRGLVLPGYKYLGPGNGLD |
| | KGEPVNAADAAALEHDKAYDQQLKAGDNPYLKYNHADAEFQERLKEDTSFGGNLGRAVFQ |
| | AKKRLLEPLGLVEEAAKTAPGKKRPVEQSPQEPDSSAGIGKSGSQPAKKKLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGGQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWPGASSWALNGRNSLMNPGPAMASHKEGEDRFFPLSGS |
| | LIFGKQGTGRDNVDADKVMITNEEEIKTTNPVATESYGQVATNHQSAQAQAQTGWVQNQG |
| | ILPGMVWQDRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVEFAVSTEGV |
| | YSEPRPIGTRYLTRNL |
| 1301 | MAADGYLPDWLEDTLSEGIRQWWKLKPGPPPPKPAERHKDDSRGLVLPGYKYLGPGNGLD |
| | KGEPVNAADAAALEHDKAYDQQLKAGDNPYLKYNHADAEFQERLKEDTSFGGNLGRAVFQ |
| | AKKRLLEPLGLVEEAAKTAPGKKRPVEQSPQEPDSSAGIGKSGSQPAKKKLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWPGASSWALNGRNSLMNPGPAMASHKEGEDRFFPLSGS |
| | LIFGKQGTGRDNVDADKVMITNEEEIKTTNPVATESYGQVATNHQSAQAQAQTGWVQNQG |
| | ILPGMVWQDRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVEFAVNTEGV |
| | YSEPRPIGTRYLTRNL |
| 1302 | MAADGYLPDWLEDTLSEGIRQRWKLKPGPPPPEPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTGTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLGRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1303 | MAADGYLPDWLEDTLSEGIRQRWKLKPGPPPPEPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNESQAVGRSSFYCLEYFPSQMLRTGNNFTFSYTFEDVPF |
| | HSSYAHSQSLGRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1304 | MAADGYLPDWLEDTLSEGIRQRWKLKPGPPPPEPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAAGRSSFYCLEYFPSQMLRTGNNFTFSYTFEDVPF |
| | HSSYAHSQSLGRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1305 | MAADGYLPDWLEDTLSEGIRQWWKLKPGPPPPKPAERHRDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSGYQLPYVLGSAHQG |
| | CLPPFPADVFMVPQYGYPTLNNGSQAVGRSSFYCLEYFPSQMLRTGNNFTFSYTFEDVPF |
| | HSSYAHSQSLDRLMNPLIDQYLYYLSTTNTPSGTTTQSRLQFSQAGASDIRDQSRNWLPG |
| | PCYRQQRVSKTSADNNNSEYSWTGATKYHLNGRDSLVNPGPAVASHKDDEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1306 | MAADGYLPDWLEDTLSEGIRQWWKLKPGPPPPKPAERHRDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVGEPVKTAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDSHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSTTNTPSGTTTQSRLQFSQAGASDIRDQSRNWLPG |
| | PCYRQQRVSKTSADNNNSEYSWTGATKYHLNGRDSLVNPGPAMASHKDNEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1307 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQGGNTQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1308 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLSLVEEPVKTAPGKKRPVEHSPAEPDSSSGTGKSGNQPARKRLNFGQTGDSD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIQDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQSGNTQAATADVNTQGV |
| | LPGMVGQDRDVYLQGPTWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1309 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPAEPDSSSGTGKSGNQPARKRLNFGQTGDSD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNSFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQSGNTQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1310 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPAEPDSSSGTGKSGNQPARKRLNFGQTGDSD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLGYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGPEKTNVDIEKVMITDEEEIRTTNPVATEQYGSVSTNLQSGNTQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1311 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRILEPLGLVEEPVKTAPGEKRPVEHSPAEPDSSSGTGKAGQQPARKRLNFGQTGDAD |
| | SVPDPQPLGQPPAAPSGLGTNTMASGSGAPMADNNEGADGVGNSSGNWYCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLVGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1312 | MAADGYLPDWLEDTLSEGIRQWWKLKPGPPPPKPAERHKDDSRGLVLPGYRYLGPFNGLD |
| | KGEPVNEADAAALEHDKAYDRQLDSGDNPYLKYNHVDAEFQERLKEDTSFGGNLGRAVFQ |
| | AKKRILEPLGLVEEPVKAAPGEKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGHFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPPMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGARYLTRNL |
| 1313 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRILEPLGLVEEPVKTAPGKKRPVEHSPAEPDSSSGTGKAGQQPARKRLNFGQTGDAD |
| | SVPDPRPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1314 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRILEPLGLVEEPVKTAPGKKRPVEHSPAEPDSSSGTGKAGQQPARKRLNFGQTGDAD |
| | SVPDPRPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1315 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRILEPLGLVEEPVKTAPGKKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGDRVI |
| | TTSTRTWALPTCNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTYLQSGNTQAATSDVNTQGV |
| | LPGMVWQDRDVYLRGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1316 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKGDTSFGGNLGRAVFQ |
| | AKKRILEPLGLVEEPVKTAPGKKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGGRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQTLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1317 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRILEPLGLVEEPVKTAPGKKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1318 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRILEPLGLVEEPVKTAPGKKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSGYQLPYVLGSAHQG |
| | CLPPFPADVFMVPQYGYLTLNNGSQAVGRSSFYCLEYFPSQMLRTGNNFTFSYTFEDVPF |
| | HSSYAHGQSLDRLMNPLIDQYLYYLSRTNTPSGTTTMSRLQFSQAGASDVRDQSRNWLPG |
| | PCYRQQRVSKTAADNNNSDYSWTGATKYHLNGRDSLVNPGPAMASHKDDEEKYFPQSGVL |
| | VFGKQDSGKTNVDIEKVMITDEEEIRTTNPAATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFVSFITQYSTGQVSVEIEWELQKENSKRWNPEIQYTSNYNKSVNVDFTVDTNGVY |
| | SEPRPIGTRYLTRNL |
| 1319 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLV |
| | NNNRGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQTNSGTLQQSRLLFSQAGPTNMSLQAKNWLPG |
| | PCYRQQRLSKQANDNNNSNFPWTAATKYHLNGRDSLVNPGPAMASHKDDEEKFFPMHGTL |
| | IFGKQGTNANDADLENVMITDEEEIRATNPVATEQYGTVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1320 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQTNSGTLQQSRLLFSQAGPTNMSLQAKNWLPG |
| | PCYRQQRLSKQANDNNNSNFPWTAATKYHLNGRDSLVNPGPAMASRKDDEEKFFPMHGTL |
| | IFGKQGTNANDADLENVMITDEEEIRATNPVATEQYGTVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1321 | MAADGYPPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQRPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQTNSGTLQQSRLLFSQAGPTNMSLQAKNWLPG |
| | PCYRQQRLSKQANDNNNSNFPWTAATKYHLNGRDSLVNPGPAMASHKDDEEKFFPMHGTL |
| | IFGKQGTNANDADLENVMITDEEEIRATNPVATEQYGTVSNNLQNSNTGPTTGTVNRQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1322 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNGGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQTNSGTLQQSRLLFSQAGPTNMSLQAKNWLPG |
| | PCYRQQRLSKQANGNNNSNFPWTAATKYHLNGRDSLVNPGPAMASHKDDEEKFFPMHGTL |
| | IFGKQGTNANDADLENVMITDEEEIRATNPVATEQYGTVSNNLQNSNTGPTTGTVNHQGA |
| | LPGMVWQDRDVYLQGPIWAKIPHTDGHFHPSPLTGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1323 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLRPGLRKPVKTAPGKKRPVEHSPVEPDSSSGTGKAGQQPARKRLNFGQTGDA |
| | DSVPDPQPLGQPPAAPSGLGSTTMATGSGAPMADNNEGADGVGNSSGNWHCDSQWLDDRV |
| | IATSTRTWALPTYNNHLYKQISSQSGACNDNHYFGYSTPWGYFDFNRFHCHFSPRDWQRL |
| | INSNWGFRPKRLNFKLFNIQVKEVTQNDGTTTIANNLTSTVQVFTDSEYQLPYVPGSAHQ |
| | GCLPPFPADVFMVPQYGYLTLNNGSQAVGRSSFYCLEYFPSQMLRTGNNFQFSYTFEDVP |
| | FHSSYAHCQSLDRLMNPLIDQYLYYLNKTQTNSGTLQQSRLLFSQAGPTNMSLQAKNWLP |
| | GPCYRQQRLSKQANDNNNCNFPWTAATKYHLNGRDSLVNPGPAMASHKDDEEKFFPMHGT |
| | LIFGKQGTNANDADLENVMITDEEEIRPTNPVATEQYGTVSNNLQNSNTGPTTGTVNHQG |
| | ALPGMVWQDRDVYLQGPIWAKIPHTDGHFHPSPLMGGFGLKHPPPQIMIKSTPVPANPPT |
| | NFSSAKFASSITQYSTGQVSVEIEWELQKENSKRWNPEIQYTSNYNKSVNVDFTVDTNGV |
| | YSEPRPIGTRYLTRNL |
| 1324 | MAADGYLPDWLEDTLSEGIRQWWKLKPGPPPPKPAERHKDGSRGLVLPGYKYLGPFNGLD |
| | KGEPVNEADAAALEHDKAYDRQLNSGDNPYLKYNHADAEFQERLKEDTSFGGNLGRAVFQ |
| | AKKRVLEPLGLVEEPVKTAPGKKRPVEHSPAEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSPFYCLEYFPSQMLRTGNNFQFSYTFEDVPF |
| | HSSYAHSQSLDRLMNPLIDQYLYYLNKTQTNSGTLQQSRLLFSQAGPTNMSLQAKNWLPG |
| | PCYRQQRLSKQANDNNNSNFPWTAATKYHLNGRDSLVNPGPAMASHKDDEEKFFPMHGTL |
| | IFGKQGTNANDADLENVMITDEEEIRTTNPVATEQYGTVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDNNGVY |
| | SEPRPIGTRYLTRNL |
| 1325 | MAADGYLPDWLEDTLSEGIRQWWKLKPGPPPPKPAERHKDDSRGLVLPGYKYLGPFNGLY |
| | KGEPVDEADAAALEHDKAYDRQLDSGDNPYLKYNHADAEFQERLKEDTSFGGNLGRAVFQ |
| | AKKRVLEPLGLVGEPVKTAPGKKRPVEHSPVEPDSSSGTGKAGNQPARKRLNFGQTGDAD |
| | SVPDPQPLGQPPAAPSGLGSTTMATGSGAPVADNNEGADGVGNSSGNWHCDSQWLGDRVI |
| | TTSTRTWALPTYNNHLYKQISSQSGASNDNHYFGYSTPWGYFDFNRFHCHFSPRDRQRLI |
| | NNNWGFRPKRLNFKLFNIQVKEVTQNDGTTTIANNLTSTVQVFTDSGYQLPYVLGLAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKEDSKRWNPEIQYTSNYNKPVNVDFTVDINGVY |
| | SEPRPIGTRYLTRNL |
| 1326 | MAADGYLPDWLEDTLSEGIRQWWKLKPGPPPPKPAERHKDDSRGLVLPGYKYLGPFNGLY |
| | KGEPVNEADAAALEHDKAYDRQLDSGDNPYLKYNHAGAEFQERLKEDTSFGGNLGRAVFQ |
| | AKKRVLEPLGLVEEPVKTAPGKKRPVEHSPVEPDSSSGTGKAGNQPARKRLNFGQTGDAD |
| | SVPDPQPLGQPPAAPSGLGSTTMATGSGAPVADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQDSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1327 | MAADGYLPDWLEDTLSEGIRQWWKLKPGPPPPKPAERHKDDSRGLVLPGYKYLGPFNGLD |
| | KGEPVNEADAAALEHDKAYDRQLESGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGSTTMASGSGAPVADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNSWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLLNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSSKFASFITQYSTGQVSVEIEWELQKENSKRWNPEIQYTSNYNKSVNVDFTVDTNGVY |
| | SEPRPIGTRYPTRNL |
| 1328 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEGPVKTAPGKKRPVEHSPAEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1329 | MAADGYLPDWLEDTLSEGTRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPAEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1330 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPAEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWAQPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQSNSGALQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHRGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1331 | MAADGYLPDWLEDTLSEGIRQWWKLKPGPPPPKPAERHQDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGHQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLNRTQSNSGTLQQSRLLFSQAGPTSMSLQAKNWLPG |
| | PCYRQQRLSKQANDNNNSNFPWTAATKYRLNGRDSLVNPGPAMASHKDDEEKFFPMHGTL |
| | IFGKQGTNANDADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1332 | MAADGYLPDWLEDTLSEGIRQWWKLKPGPPPPKPAERHQDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGHQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRPSFYCLEYFPSQMLRTGNNFTFSYTFEDVPF |
| | HSSYAHSQSLDRLMNPLIDQYLYYLNRTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1333 | MAADGYLPDWLEDTLSEGIRQWWKLKPGPPPPKPAERHQDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGHQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRPSFYCLEYFPSQMLRTGNNFTFSYTFEDVPF |
| | HSSYAHSQSLDRLMNPLIDQYLYYLNRTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1334 | MAADGYLPDWLEDTLSEGIRQWWKLKPGPPPPKLAERHQDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGHQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFTVPQYGYLTLNNGSQAVGRSSFYCLEYFPSQMLRTGNNLTFSYTFEDVPF |
| | HSSYAHSQSLDRLMNPLIDQYLYYLNRTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKNTPVPANPPTN |
| | YSSAKFASFITQYSTGQVSVEIEWELRKENSKRWNPEIQYTSNYNKSVNVDFTVDTNGVY |
| | SEPRPIGTRYLTRNL |
| 1335 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEQSPQEPDSSSGIGKTGQQPAKKRLNFGQTGDSE |
| | SVPDPQPLGEPPATPAAVGPTTMASGGGAPMADNNEGADGVGNASGNWHCDSTWLGDRVI |
| | TTSTRTWALPTYNNHLYKQISSTSTGASNDNHYFGYGTPWGYFDFNRFHCHFSPRDWQRL |
| | INNNWGFRPKRLNFKLFNIQVEEVTTNDGVTTIANNLTSTVQVFSDSEYQLPYVLGSAHQ |
| | GCLPPFPADVFMIPQYGYLTLNNGSQAVGRSSFYCLEYFPSQMLRTGNNFTFSYTFEEVP |
| | FHSSYAHSQSLDRLMNPLIDQYLYYLNRTQNQSGSAQNKDLLFSRGSPAGMSVQPKNWLP |
| | GPCYRQQRVSKTKTDNNNSNFTWTGASKYNLNGRESIINPGTAVASHKDDEDKFFPMSGV |
| | MIFGKESAGASSTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIKNTPVPANPPA |
| | EFSATKFASFITQYSTGQVSVEIEWELQKENSKRWNPEVQYTSNYAKSANVDFTVDNNGL |
| | YTEPRPIGTRYLTRPL |
| 1336 | MAADGYLPDWLEDTLSEGIRQWWKLRPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEGAETAPGKKRPVEQSPQGPDSSSGIGKTGQQPAKKRLNFGQTGDSE |
| | SVPDPQPLGEPPATPAAVGPTTMASGGGAPMADNNEGADGVGNASGNWHCDSTWLGDRVI |
| | TTSTRTWALPTYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQLPYVLGSAHQ |
| | GCLPPFPADVFMIPQYGYLTLNNGSQAVGRSSFYCLEYFPSQMLRTGNNFTFSYTFEEVP |
| | FHSSYAHSQSLDRLMNPLIDQYLYYPNRTQNQSGSAQNKDLLFSRGSPAGMSVQPKNWLP |
| | GPCYRQQRVSKTKTDNNNSNFTWTGASKYNLNGRESIINPGTAMASHKDDEDKFFPMSGV |
| | MIFGKESAGASNTALDNVMITDEEEIKATNPVATERFGTVAVNFQSSSTDPATGDVHAMG |
| | ALPGMVWQGRDVYLQGPIWAKIPHTDGHFHPSPLMGGFGLKNPPPQILIKNTPVPANPPA |
| | EFSATKFASFITQYSTGQVSVEIEWELQKENSKRWNPEVQYTSNYAKSANVDFTVDNNGL |
| | YTEPRPIGTRYLTRPL |
| 1337 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEGAKTAPGKKRPVEQSPQEPDSPSGIGKTGQQPAKKRLNFGQTGDSE |
| | SVPDPQPLGEPPATPAAVGPTTMASGGGAPMADNNEGADGVGNASGNWHCDSTWLGDRVI |
| | TTSTRTWALPTYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQLPYVLGSAHQ |
| | GRLPPFPADVFMIPQYGYLTLNNGSQAVGRSSSYCLEYFPSQMLRTGNNFTFSYTFEEVP |
| | LHSSCAHSQSLDRLMNPLIDQYLYYLNRTQNQSGSAQNRDLLFSRGSPAGMSVQPKNWLP |
| | GPCYRQQRVSKTKTDNNNSNFTWTGASKYNLNGRESIINPGTAMASHKDDEDKFFPMSGV |
| | MIFGKESAGASNTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIKNTPVPANPPA |
| | EFSATKFASFITQYSAGQVSVEIEWELQKENSKRWNPEVQYTSNYAKSANVDFTVDNNGL |
| | YTEPRPIGTRYLTRPL |
| 1338 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYPRYNHADAEFQERLQEDTPFGGNLGRAVFQ |
| | AKKRVLEPLGLVEEAAKTAPGKKRPVEPSPQRSPDSSTGIGKKGQQPAKKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSASTGASNDNHYFGYSTPWGYFDFNRFHCHFSPRDWQR |
| | LINNNWGFRPKRLNFKLFNIQVKEVTTNDGVTTIANNLTSTVQVFSDSEYQLPYVLGSAH |
| | QGCLPPFPADVFMIPQYGYLTLNNGSQAVGRSSFYCLEYFPSQMLRTGNNFTFSYTFEEV |
| | PLHSSYAHSQSLDRLMNPLIVQYLYYLNRTQNQSGSAQNKDLLFSRGSPAGMSVQPKNWL |
| | PGPCYRQQRVSKTKTDNNNSNFTWTGASKYNLNGRESIINPGTAMASHKDDEDKFFPMSG |
| | VMIFGKESAGASNTALDNVMITDEEEIKATNPVATERFGTVAVNFQSSSTDPATGDVHAM |
| | GALPGMVWQDRDVYLQGPIWAKIPHTDGHFHPSPLMGGFGLKNPPPQILIKNTPVPANPP |
| | AEFSATKFASFITQYSTGQVSVEIEWELQKENSKRWNPEVQYTSNYAKSASVDFTVDNNG |
| | LYTEPRPIGTRYLTRPL |
| 1339 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNCHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1340 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEGAKTAPGKKRPVEQSPQEPDSSSGIGKTGQQPAKKRLNFGQTGDSE |
| | SVPDPQPLGEPPATPAAVGPTTMASGGGAPMADNNEGADGVGNASGNWHCDSTWLGDRVI |
| | TTSTRTWALPTYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQLPYVLGSAHQ |
| | GCLPPFPADVFMIPQYGYLTLNNGSQAVGRSSFYCLEYFPSQMLRTGNNFTFSYTFEEVP |
| | FHSSYAHSQSLDRLMNPLIDQYLYYLNRTQNQSGSAQNKDLLFSRGSPAGMSVQPKNWLP |
| | GPCYRQQRVSKTKTDNNNSNFTWTGASKYNLNGRESIINPGTAMASHKDDEDKFFPMSGV |
| | MIFGKESAGASNTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIKNTPVPANPPA |
| | EFSATKFASFITQYSTGQVSVEIEWELQKENSKRWNPEVQYTSNYAKSANVDFTVDNNGL |
| | YTEPRPIGTRYLTRPL |
| 1341 | MAADGYLPDWLEDNLSEGIREWWDLKPGAPKPKANQQKQDDGRGLVLPGYKYLGPFNGLD |
| | KGEPVNAADAAALEHDKAYDQQLEAGDNPYLRYNHADAEFQERLQEDTSFGGNLGRAVFQ |
| | AKKRVLEPLGLVEEGAKTAPGKKRPVEQSPQEPDSSSGIGKKGQQPARKRLNFGQTGDSE |
| | SVPDPQPLGEPPAAPSGVGPNTMAAGGGAPMADNNEGADGVGSSSGNWHCDSTWLGDRVI |
| | TTSTRTWALPTYNNHLYKQISNGTSGGATNDNTYFGYSTPWGYFDFNRFHCHFSPRDWQR |
| | LINNNWGFRPKRLSFKLFNIQVKEVTQNEGTKTIANNLTSTIQVFTDSEYQLPYVLGSAH |
| | QGCLPPFPADVFMIPQYGYLTLNNGSQAVGRSSFYCLEYFPSQMLRTGNNFQFTYTFEDV |
| | PFHSSYAHSQSLDRLMNPLIDQYLYYLSRTQTTGGTANTQTLGFSQGGPNTMANQAKNWL |
| | PGPCYRQQRVSTTTGQNNNSNFAWTAGTKYHLNGRNSLANPGIAMATHKDDEERFFPVTG |
| | SCFWQQNAARDNADYSDVMLTSEEEIKTTNPVATEEYGIVADNLQQQNTAPQIGTVNSQG |
| | ALPGMVWQNRDVYLQGPIWAKIPHTDGNFHPSPLMGGFGLKHPPPQILIKNTPVPADPPT |
| | TFNQSKLNSFITQYSTGQVSVEIEWELQKENSKRWNPEIQYTSNYYKSTSVDFAVNTEGV |
| | YSEPRPIGTRYLTRNL |
| 1342 | MAADGYLPDWLEDTLSEGIRQRWKLKPGPPPPEPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLGRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1343 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLVDQYLYYLNKTQTNSGTLQQSRLLFSQAGPTNMSLQAKNWLPG |
| | PCYRQQRLSKQANDNNNSNFPWTAATKYHLNGRDSLVNPGPAMASHKDDEEKFFPMHGTL |
| | IFGKQGTNANDADLENVMITDEEEIRTTNPVATEQYGTVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1344 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHPPVEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQTNSGTLQQSRLLFSQAGPTNMSLQAKNRLPG |
| | PCYRQQRLSKQANDNNNSNFPWTAATKYHLNGRDSLVNPGPAMASHKDDEEKFFPMHGTL |
| | IFGKQGTNANDADLENVMITDEEEIRTTNPVATEQYGTVSNNLQNSNTGPTTGTVNHQGA |
| | LPGMVWQDRDVYLQGPIWAKIPHTDGHFHPSPLVGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1345 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPAEPDSSSGTGKAGQQPARKRLNFGQTGDAD |
| | SVPDPQPLRQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNRTQTASGTQQSRLLFSQAGPTSMSLQAKNWLPGP |
| | CYRQQRLSKQANDNNNSNFPWTGATKYYLNGRDSLVNPGPAMASHKDDEEKFFPMHGTLI |
| | FGKEGTNATNAELENVMITDEEEIRTTNPVATEQYGYVSNNLQNSNTAASTETVNHQGAL |
| | PGMVWQDRDVYLQGPIWAKIPHTDGHFHPSPLMGGFGLKHPPPQIMIKNTPVPANPPTNF |
| | SSAKFASFITQYSTGQVSVEIEWELQKENSKRWNPEIQYTSNYNKSVNVDFTVDINGVYS |
| | EPRPIGTRYLTRNL |
| 1346 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPAEPDSSSGTGKAGQQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLECFPSQMLRTGNNFQFSYTFEDVPF |
| | HSSYAHSQSLDRLMNPLIDQYLYYLNRTQTASGTQQSRLLFSQAGPTSMSLQAKNWLPGP |
| | CYRQQRLSKQANDNNNSNFPWTGATKYHLNGRDSLVNPGPAMASHKDDEEKFFPMHGTLI |
| | FGKEGTNATNAELENVMITDEEEIRTTNPVATEQYGYVSNNLQNSNTAASTETVNHQGAL |
| | PGMVWQDRDVYLQGPIWAKIPHTDGHFHPSPLMGGFGLKHPPPQIMIKNTPVPANPPTNF |
| | SSAKFASFITQYSTGQVSVEIEWELQKENSKRWNPEIQYTSNYNKSVNVDFTVDTNGVYS |
| | EPRPIGTRYLTRNL |
| 1347 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPAEPDSSSGTGKAGQQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCRFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQGLDRLMNPLIDQYLYYLNRTQTASGTQQSRLLFSQAGPTSMSLQAKNWLPGP |
| | CYRQQRLSKQANDNNNSNFPWTGATKYHLNGGDSLVNPGPAMASHKDDEEKFFPMHGTLI |
| | FGKEGTNATNAELENVMITDEEEIRTTNPVATEQYGYVSNNLQNSNTAASTETVNHQGAL |
| | PGMVWQDRDVYLRGPIWAKIPHADGHFHPSPLMGGFGLKHPPPQIMIKNTPVPANPPTNF |
| | SSAKFASFITQYSTGQVSVEIEWELQKENSKRWNPEIQYTSNYNKSVNVDFTVDINGVYS |
| | EPRPIGTRYLTRNL |
| 1348 | MAADGYLPDWLKDTLSEGIRQWWKLKPGPPPPKPAERHKDDSGGLVLPGYKYLGPFNGLD |
| | KGEPVNEADAAALEYDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGQQPARKRLNFGQTGDAD |
| | SVPDPQPLGQPPAAPSGLGTNTMATGSGAPMADNNEGADGVGNSSGSWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVPGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTAADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQSGNTQAATADVNTQGV |
| | LPGMVWQDRDVHLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1349 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVGEPVKTAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSGYAHSQSLDRLMNPLIDQYLYYLSTTNTPSGTTTQSRLQFSQAGASDIRDQSRNWLPG |
| | PCYRQQRVSKTSADNNNSEYSWTGATKYHLNGRDSLVNPGPAMASHKDNEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1350 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVGEPVKTAPGKKRPVEHSPVEPDSSSGTGKAGQQPARKRLNFGQTGDAD |
| | SVPDPQPLGQPPAAPSGLGTNTMATGSGAPMADNNEGADGVGNSSGNRHCDSTWMGDRVI |
| | TTSTRTWALPTYNNHLYRQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLSNGSQAVGRSSFYCPEYFPSQMLRTGNNFTFSYTFEDVPF |
| | HSSYAHSQSLDRLMNPLIDQYLYYLSTTNTPSGTTTQSRLQFSQAGASDIRDQSRNWLPG |
| | PCYRQQRVSKTSADNNNSEYSWTGATKYHLNGRDSLVNPGPAMASHKDNEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGPKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1351 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYNHADAEFQERLKEDTSFGGNLGRAVFQ |
| | AKKRVLEPLGLVEEPVKTAPGKKRPVEHSPVEPDSSSGTGKAGNQPARKRLNFGQTGDAD |
| | SVPDPQPLGQPPASPSGLGTNTMATGSGAPMADNNEGADGVGNSSGNWHCDSTWMGDRVV |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDLEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTAADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1352 | MAADGYLPDWLEDTLSEGIRQWWKLKPGPPPKPAERHKDDSRGLVLPGYKYLGPFNGLDK |
| | GEPVNEADAAALEHDKAYDRQLDSGDNPYLKYNHADAEFQERLKEDTSFGGNLGRAVFQA |
| | KKRVLEPLGLVEEPVKTAPGKKRPVEHSPVEPDSSSGTGKAGNQPARKRLNFGQTGDADS |
| | VPDPQPLGQPPAAPSGLGTNTMATGSGAPMADNNEGADGVGNSSGDWHCDSTWMGDRVIT |
| | TSTRTWALPTYNNHLYKQISSQSGASNDNHYFGYSTPWGYFDFNRFHCHFSPRDWQRLIN |
| | NNWGFRPKRLNLKLFNIQVKEVTQNDGTTTIANNLTSTVQVFTDLEYQLPYVLGSAHQGC |
| | LPPFPADVFMVPQYGYLTLNNGSQAVGRSSFYCLEYFPSQMLRTGNNFTFSYTFEDVPFH |
| | SSYAHSQSLDRLMNPLIDQYLYYLSRTNTPSGTTTQSRLQFSQAGASDIRDQSRNWLPGP |
| | CYRQQRVSKTAADNNNGEYSWTGATKYHLNGRDSLVNPGPAMASHKDDEEKFFPQSGVLI |
| | FGKQGSEKTNVDIEKVMITDEEEIRTTNPVATEQYGSVSTNLQSGNTRAATSDVNTQGVL |
| | PGMVWQDRDVYLQGPIWAKIPHTDGHFHPSPLMGGFGLKHPPPQILIKNTPVPANPSTTF |
| | SAAKFASFITQYSTGQVSVEIEWELQKENSKRWNPEIQYTSNYNKSVNVDFTVDINGVYS |
| | EPRPIGTRYLTRNL |
| 1353 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYLKYDHADAEFQERLKEDTSFGGNLGRAVFQ |
| | AKKRVLEPLGLVEEPVKTAPGKKRPVEHSPVEPDSSSGTGKAGNQPARKRLNFGQTGDAD |
| | SVPDPQPLGQPPAAPSGLGTNTMATGSGAPMADNNDGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVREVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWIGATKYHLNGRDSLVNPGPAMASHKDDEEKFFPQSGVL |
| | IFGKQGSEKTNVDIEKVMITDEEEIRTTNPVATEQYGSVSTNLQSGNTQASTADVNTQGV |
| | LPGMVWQDRDVYLQGPIWAKIPHTDGHFHPSPLMGGFGLKHPPPQILIKNTPVPANPSTT |
| | FSAAKFASFITQYSTGRVSVEIEWELQKENSKRWNPEIQYTSNYNKSVNVDFTVDINGVY |
| | SEPRPIGTRYLTRNL |
| 1354 | MAADGYLPDWLEDTLSEGIRQWWKLKPGPPPPKPAERHKDDSRGLVLPGYKYLGPFNGLD |
| | KGEPVNEADAAALEHDKAYDRQLDSGDNPYPKYNHADAEFQERLKEDTSFGGNLGRAVFQ |
| | AKKRVLEPLGLVEEPVKTAPGKKRPVEHSPAEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1355 | MAADGYLPDWLEDTLSEGIRQWWKLKPGPPPPKPAERHKDDSRGLVLPGYKYLGPFNGLD |
| | KGEPVNEADAAALEHDKAYDRQLDGGDNPYLKYNHADAEFQERLKEDTSFGGNLGRAVFQ |
| | AKKRVLEPLGLVEEPVKTAPGKKRPVEHSLAEPDSSSGTGKAGQQPARRRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYRQISSQSGASNDNHYFGYSTPWGYFDFNRFHCHFSPRDWQRLI |
| | NNNWGSRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLVDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1356 | MAADGYLPDWLEDNLSEGIREWWDLKPGAPKPKANQQKQDDGRGLVLPGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSAGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTETIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSCAHSQSSDRLMNPLIDQYLYYLSRTRSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1357 | MAADGYLPDWLEDNLSEGIREWWDLKPGAPKPKANQQKQDDGRGLVLLGYKYLGPFNGLD |
| | KGEPVNAADAAALEHDKAYDQQLKAGDNPYLRYNHADAEFQERLQEDTSFGGNLGRAVFQ |
| | AKKRVLEPLGLVEEAAKTAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSGYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |

TABLE 6
VP2 capsid proteins

| SEQ | |
|---|---|
| ID | |
| NO.: | VP2 Capsid Protein Sequence |
| 1358 | MAPGKKRPVEQSPQEPDSSAGIGKSGAQPAKKRLNFGQTGDTESVPDPQPIGEPPAAPSG |
| | VGSLTMASGGGAPVADNNEGADGVGSSSGNWHCDSQWLGDRVITTSTRTWALPTYNNHLY |
| | KQISNSTSGGSSNDNAYFGYSTPWGYFDFNRFHCHFSPRDWQRLINNNWGFRPKRLNFKL |
| | FNIQVKEVTDNNGVKTIANNLTSTVQVFTDSDYQLPYVLGSAHEGCLPPFPADVFMIPQY |
| | GYLTLNDGSQAVGRSSFYCLEYFPSQMLRTGNNFQFSYEFENVPFHSSYAHSQSLDRLMN |
| | PLIDQYLYYLSKTINGSGQNQQTLKFSVAGPSNMAVQGRNYIPGPSYRQQRVSTTVTQNN |
| | NSEFAWAAASSWALNGRNSLMNPGPAMASHKEGEDRFFPLSGSLIFGKQGTGRDNVDADK |
| | VMITNEEEIKTTNPVATESYGQVATNHQGQSGRGDLGLSAQAAQTGWVQNQGILPGMVWQ |
| | DRDVYLQGPIWAKIPHTDGNFHPSPLMGGFGMKHPPPQILIKNTPVPADPPTAFNKDKLN |
| | SFITQYSTGQVSVEIEWELQKENSKRWNPEIQYTSNYYKSNNVEFAVNTEGVYSEPRPIG |
| | TRYLTRNL |
| 1359 | MAPGKKRPVEQSPQEPDSSAGIGKSGAQPAKKRLNFGQTGDTESVPDPQPIGEPPAAPSGV |
| | GSLTMASGGGAPVADNNEGADGVGSSSGNWHCDSQWLGDRVITTSTRTWALPTYNNHLYK |
| | QISNSTSGGSSNDNAYFGYSTPWGYFDFNRFHCHFSPRDWQRLINNNWGFRPKRLNFKLF |
| | NIQVKEVTDNNGVKTIANNLTSTVQVFTDSDYQLPYVLGSAHEGCLPPFPADVFMIPQYG |
| | YLTLNDGSQAVGRSSFYCLEYFPSQMLRTGNNFQFSYEFENVPFHSSYAHSQSLDRLMNP |
| | LIDQYLYYLSKTINGSGQNQQTLKFSVAGPSNMAVQGRNYIPGPSYRQQRVSTTVTQNNN |
| | SEFAWPGASSWALNGRNSLMNPGPAMASHKEGEDRFFPLSGSLIFGKQGTGRDNVDADKV |
| | MITNEEEIKTTNPVATESYGQVATNHQSGQSGRGDLGLSAQAATGWVQNQGILPGMVWQD |
| | RDVYLQGPIWAKIPHTDGNFHPSPLMGGFGMKHPPPQILIKNTPVPADPPTAFNKDKLNS |
| | FITQYSTGQVSVEIEWELQKENSKRWNPEIQYTSNYYKSNNVEFAVNTEGVYSEPRPIGT |
| | RYLTRNL |
| 1360 | MAPGKKRPVEQSPQEPDSSAGIGKSGAQPAKKRLNFGQTGDTESVPDPQPIGEPPAAPSG |
| | VGSLTMASGGGAPVADNNEGADGVGSSSGNWHCDSQWLGDRVITTSTRTWALPTYNNHLY |
| | KQISNSTSGGSSNDNAYFGYSTPWGYFDFNRFHCHFSPRDWQRLINNNWGFRPKRLNFKL |
| | FNIQVKEVTDNNGVKTIANNLTSTVQVFTDSDYQLPYVLGSAHEGCLPPFPADVFMIPQY |
| | GYLTLNDGSQAVGRSSFYCLEYFPSQMLRTGNNFQFSYEFENVPFHSSYAHSQSLDRLMN |
| | PLIDQYLYYLSKTINGSGQNQQTLKFSVAGPSNMAVQGRNYIPGPSYRQQRVSTTVTQNN |
| | NSEFAWPGASSWALNGRNSLMNPGPAMASHKEGEDRFFPLSGSLIFGKQGTGRDNVDADK |
| | VMITNEEEIKTTNPVATESYGQVATNHQGQSGRGDAVGVAQAAQTGWVQNQGILPGMVWQ |
| | DRDVYLQGPIWAKIPHTDGNFHPSPLMGGFGMKHPPPQILIKNTPVPADPPTAFNKDKLN |
| | SFITQYSTGQVSVEIEWELQKENSKRWNPEIQYTSNYYKSNNVEFAVNTEGVYSEPRPIG |
| | TRYLTRNL |
| 1361 | MAPGKKRPVEQSPQEPDSSAGIGKSGAQPAKKRLNFGQTGDTESVPDPQPIGEPPAAPSGV |
| | GSLTMASGGGAPVADNNEGADGVGSSSGNWHCDSQWLGDRVITTSTRTWALPTYNNHLYK |
| | QISNSTSGGSSNDNAYFGYSTPWGYFDFNRFHCHFSPRDWQRLINNNWGFRPKRLNFKLF |
| | NIQVKEVTDNNGVKTIANNLTSTVQVFTDSDYQLPYVLGSAHEGCLPPFPADVFMIPQYG |
| | YLTLNDGSQAVGRSSFYCLEYFPSQMLRTGNNFQFSYEFENVPFHSSYAHSQSLDRLMNP |
| | LIDQYLYYLSKTINGSGQNQQTLKFSVAGPSNMAVQGRNYIPGPSYRQQRVSTTVTQNNN |
| | SEFAWAAASSWALNGRNSLMNPGPAMASHKEGEDRFFPLSGSLIFGKQGTGRDNVDADKV |
| | MITNEEEIKTTNPVATESYGQVATNHQGQSGRGDAVGVAQAAQTGWVQNQGILPGMVWQD |
| | RDVYLQGPIWAKIPHTDGNFHPSPLMGGFGMKHPPPQILIKNTPVPADPPTAFNKDKLNS |
| | FITQYSTGQVSVEIEWELQKENSKRWNPEIQYTSNYYKSNNVEFAVNTEGVYSEPRPIGT |
| | RYLTRNL |
| 1362 | MAPGKKRPVEQSPQEPDSSAGIGKSGAQPAKKRLNFGQTGDTESVPDPQPIGEPPAAPSG |
| | VGSLTMASGGGAPVADNNEGADGVGSSSGNWHCDSQWLGDRVITTSTRTWALPTYNNHLY |
| | KQISNSTSGGSSNDNAYFGYSTPWGYFDFNRFHCHFSPRDWQRLINNNWGFRPKRLNFKL |
| | FNIQVKEVTDNNGVKTIANNLTSTVQVFTDSDYQLPYVLGSAHEGCLPPFPADVFMIPQY |
| | GYLTLNDGSQAVGRSSFYCLEYFPSQMLRTGNNFQFSYEFENVPFHSSYAHSQSLDRLMN |
| | PLIDQYLYYLSKTINGSGQNQQTLKFSVAGPSNMAVQGRNYIPGPSYRQQRVSTTVTQNN |
| | NSEFAWAAASSWALNGRNSLMNPGPAMASHKEGEDRFFPLSGSLIFGKQGTGRDNVDADK |
| | VMITNEEEIKTTNPVATESYGQVATNHQSGQSGRGDAVGVAQAATGWVQNQGILPGMVWQ |
| | DRDVYLQGPIWAKIPHTDGNFHPSPLMGGFGMKHPPPQILIKNTPVPADPPTAFNKDKLN |
| | SFITQYSTGQVSVEIEWELQKENSKRWNPEIQYTSNYYKSNNVEFAVNTEGVYSEPRPIG |
| | TRYLTRNL |
| 1363 | MAPGKKRPVEQSPQEPDSSAGIGKSGAQPAKKRLNFGQTGDTESVPDPQPIGEPPAAPSG |
| | VGSLTMASGGGAPVADNNEGADGVGNASGNWHCDSQWLGDRVITTSTRTWALPTYNNHLY |
| | KQISSASTGATNDNTYFGYSTPWGYFDFNRFHCHFSPRDWQRLINNNWGFRPKRLNFKLF |
| | NIQVKEVTDNNGVKTIANNLTSTVQVFTDSDYQLPYVLGSAHEGCLPPFPADVFMIPQYG |
| | YLTLNDGSQAVGRSSFYCLEYFPSQMLRTGNNFQFSYEFENVPFHSSYAHSQSLDRLMNP |
| | LIDQYLYYLSKTINGSGQNQQTLKFSVAGPSNMAVQGRNYIPGPSYRQQRVSTTVTQNNN |
| | SEFAWPGASSWALNGRNSLMNPGPAMASHKEGEDRFFPLSGSLIFGKQGTGRDNVDADKV |
| | MITNEEEIKTTNPVATESYGQVATNHQGQSGRGDLGLSAQAAQTGWVQNQGILPGMVWQD |
| | RDVYLQGPIWAKIPHTDGNFHPSPLMGGFGMKHPPPQILIKNTPVPADPPTAFNKDKLNS |
| | FITQYSTGQVSVEIEWELQKENSKRWNPEIQYTSNYYKSNNVEFAVNTEGVYSEPRPIGT |
| | RYLTRNL |
| 1364 | MAPGKKRPVEPSPQRSPDSSTGIGKTGQQPAKKRLNFGQTGDTESVPDPQPIGEPPAAPA |
| | AVGPTTMASGGGAPMADNNEGADGVGNASGNWHCDSTWLGDRVITTSTRTWALPTYNNHL |
| | YKQISNGTSGGATNDNTYFGYSTPWGYFDFNRFHCHFSPRDWQRLINNNWGFRPKRLNFK |
| | LFNIQVKEVTDNNGVKTIANNLTSTVQVFTDSDYQLPYVLGSAHEGCLPPFPADVFMIPQ |
| | YGYLTLNDGSQAVGRSSFYCLEYFPSQMLRTGNNFQFSYEFENVPFHSSYAHSQSLDRLM |
| | NPLIDQYLYYLSKTINGSGQNQQTLKFSVAGPSNMAVQGRNYIPGPSYRQQRVSTTVTQN |
| | NNSEFAWPGASSWALNGRNSLMNPGPAMASHKEGEDRFFPLSGSLIFGKQGTGRDNVDAD |
| | KVMITNEEEIKTTNPVATESYGQVATNHQGQSGRGDLGLSAQAAQTGWVQNQGILPGMVW |
| | QDRDVYLQGPIWAKIPHTDGNFHPSPLMGGFGMKHPPPQILIKNTPVPADPPTAFNKDKL |
| | NSFITQYSTGQVSVEIEWELQKENSKRWNPEIQYTSNYYKSNNVEFAVNTEGVYSEPRPI |
| | GTRYLTRNL |
| 1365 | MAPGKKRPVEQSPQEPDSSSGIVKTGQQPAKKRLNFGQTGDTESVPDPQPLGEPPATPSG |
| | VGSLTMASGGGAPMADNNEGADGVGNASGNWHCDSTWLGDRVITTSTRTWALPTYNNHLY |
| | KQISNGTSGGATNDNAYFGYSTPWGYFDFNRFHCHFSPRDWQRLINNNWGFRPKRLNFKL |
| | FNIQVKEVTDNNGVKTIANNLTSTVQVFTDSDYQLPYVLGSAHEGCLPPFPADVFMIPQY |
| | GYLTLNDGSQAVGRSSFYCLEYFPSQMLRTGNNFQFSYEFENVPFHSSYAHSQSLDRLMN |
| | PLIDQYLYYLSKTINGSGQNQQTLKFSVAGPSNMAVQGRNYIPGPSYRQQRVSTTVTQNN |
| | NSEFAWPGASSWALNGRNSLMNPGPAMASHKEGEDRFFPLSGSLIFGKQGTGRDNVDADK |
| | VMITNEEEIKTTNPVATESYGQVATNHQGQSGRGDLGLSAQAAQTGWVQNQGILPGMVWQ |
| | DRDVYLQGPIWAKIPHTDGNFHPSPLMGGFGMKHPPPQILIKNTPVPADPPTAFNKDKLN |
| | SFITQYSTGQVSVEIEWELQKENSKRWNPEIQYTSNYYKSNNVEFAVNTEGVYSEPRPIG |
| | TRYLTRNL |
| 1366 | MAPGKKRPVEQSPQEPDSSSGIGKTGQQPAKKRLNFGQTGDSESVPDPQPLGEPPATPAA |
| | VGPTTMASGGGAPVADNNEGADGVGSSSGNWHCDSQWLGDRVITTSTRTWALPTYNNHLY |
| | KQISNSTSGGSSNDNAYFGYSTPWGYFDFNRFHCHFSPRDWQRLINNNWGFRPKRLNFKL |
| | FNIQVKEVTDNNGVKTIANNLTSTVQVFTDSDYQLPYVLGSAHEGCLPPFPADVFMIPQY |
| | GYLTLNDGSQAVGRSSFYCLEYFPSQMLRTGNNFQFSYEFENVPFHSSYAHSQSLDRLMN |
| | PLIDQYLYYLSKTINGSGQNQQTLKFSVAGPSNMAVQGRNYIPGPSYRQQRVSTTVTQNN |
| | NSEFAWPGASSWALNGRNSLMNPGPAMASHKEGEDRFFPLSGSLIFGKQGTGRDNVDADK |
| | VMITNEEEIKTTNPVATESYGQVATNHQGQSGRGDLGLSAQAAQTGWVQNQGILPGMVWQ |
| | DRDVYLQGPIWAKIPHTDGNFHPSPLMGGFGLKHPPPQILIKNTPVPADPPAEFSATKFA |
| | SFITQYSTGQVSVEIEWELQKENSKRWNPEVQYTSNYAKSANVDFTVDNNGLYTEPRPIG |
| | TRYLTRPL |
| 1367 | MAPGKKRPVEPSPQRSPDSSTGIGKTGQQPAKKRLNFGQTGDS |
| | ESVPDPQPIGEPPAGPSGLGSGTMAAGGGAPMADNNEGADGVGSSSGNWHCDSAWLGDRV |
| | ITTSTRPWALPTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSEYQLPYVLGSA |
| | HQGCPPPFPADVFMIPQYGYLTLNNGSQAVGRSSFYCLEYFPSQMRRTGNNFEFSYQFED |
| | VPFHSSYAHSQSLDRLMNPLIDQYLYYLSRTQSTGGTAGTQQLLFSQAGPNNMSAQAKNW |
| | LPGPCYRQQRVSTTLSQNNNSNFAWTGATKYHLNGRDSLVNPGVAMATHKDDEERFFPSS |
| | GVLMFGKQGAGKDNVDYSSVMLTSEEEIKTTNPVATEQYGVVADNLQQQNAAPIVGAVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1368 | MAPGKKRPVEPSPQRSPDSSTGIGKTGQQPAKKRLNFGQTGDS |
| | ESVPDPQPIGEPPAGPSGLGSGTMAAGGGAPMADNNEGADGVGSSSGNWHCDSAWLGDRV |
| | ITTSTRPWALPTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSEYQLPYVLGSA |
| | HQGCPPPFPADVFMIPQYGYLTLNNGSQAVGRSSFYCLEYFPSQMRRTGNNFEFSYQFED |
| | VPFHSSYAHSQSLDRLMNPLIDQYLYYLSRTQSTGGTAGTQQLLFSQAGPNNMSAQAKNW |
| | LPGPCYRQQRVSTTLSQNNNSNFAWTGATKYHLNGRDSLVNPGVAMATHKDDEERFFPSS |
| | GVLMFGKQGAGKDNVDYSSVMLTSEEEIKTTNPVATEQYGVVADNLQQQNAAPIVGAVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1369 | MAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQSNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGLGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1370 | MAPGKKRPVEPSPQRSPDSSTGIGKKGQRPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKPFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLHYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1371 | MAPGKKRPVEPSPQRSPDSSTGIGKKGQQPAKKRLSFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNSEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1372 | MAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1373 | MAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1374 | MAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1375 | MAPGKKRPVEPPPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTVANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1376 | MAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQDEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSARAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQANTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNSE |
| | GTYSEPRPIGTRYLTRNL |
| 1377 | MAPGKKRPVEPDSSSGIGKSGQQPAKKRLNFGQTGDSESVPDP |
| | QPLSEPPAGPSGLGSGTMAAGGGAPMADNNEGADGVGNVSGNWHCDSTWLGDRVITTSTR |
| | TWALPTYNNHLYKQISNGTSGGSSNDNTYFGYSTPWGYFDFNRFHCHFSPRDWQRLINNN |
| | WGFRPKKLNFKLFNIQVKEVTQNEGTKTIANNLTSTVQVFTDSEYQLPYVLGSAHQGCLP |
| | PFPADVFMIPQYGYLTLNNGSQAVGRSSFYCLEYFPSQMLRTGNNFTFSYTFEDVPFHSS |
| | YAHSQSLDRLMNPLIDQYLYYLSRTQTNGTNATQTLLFAQAGPQNMSAQAKNWLPGPCYR |
| | QQRVSTTVSQNNNSNFTWTGATKYHLNGRDSLVSPGVAMATHKDDEERFFPSSGVLMFGK |
| | QGAGKDNVEYTNVMLTSEEEIKTTNPVATEQYGVVADNLQQTNSAPIVGAVNSQGALPGM |
| | VWQNRDVYLQGPIWAKIPHTDGNFHPSPLMGGFGLKHPPPQILIKNTPVPADPPVNFTDA |
| | KLASFITQYSTGQVSVEIEWELQKENSKRWNPEIQYTSNYYKSANVDFAVNADGVYSEPR |
| | PIGTRYLTRNL |
| 1378 | MAPGKKRPVEPDSSSGIGKSGQQPAKKRLNFGPTGDSESVPDP |
| | QPLSEPPAGPSGLGSGTMAAGGGAPMADNNEGADGVGNVSGNWHCDSTWLGDRVITTSTR |
| | TWALPTYNNHLYKQISNGTSGGSSNDNTYFGYSTPWGYFDFNRFHCHFSPRDWQRLINNN |
| | WGFRPKKLNFKLFNIQVKEVTQNEGTKTTANNLTSTVQVFTDSEYQLPYVLGSAHQGCLP |
| | PFPADVFMIPQYGYLTLNNGSQAVGRSSFYCLEYFPSQMLRTGNNFTFSYTFEDVPFHSS |
| | YAHSQSLDRLMNPLIDQYLYYLSRTQTNGTNATQTLLFAQAGPQNMSAQAKNWLPGPCYR |
| | QQRVSTAVSQNNNSNFTWTGATKYHLNGRDSLVNPGVAMATHKDDEERFFPSSGVLMFGK |
| | QGAGKDNVEYTNVMLTSEEEIKTTNPVATEQYGVVADNLQQTNSAPIVGAVNSQGALPGM |
| | VWQNRDVYLQGPIWAKIPHTDGNFHPSPLMGGFGLKHPPPQILIKNTPVPADPPVNFTDA |
| | KLASFITQYSTGQVSVEIEWELQKENSKRWNPEIQYTSNYYKSANVDFAVNADGVYSEPR |
| | PIGTRYLTRNL |
| 1379 | MAPGKKRPVEPDSSSGIGKSGRQPAKKRLNFGQTGDSESVPDP |
| | QPLSEPPAGPSGLGSGTMAAGGGAPMADNNEGADGVGNASGNWHCDSTWLGDRVITTSTR |
| | TWALPTYNNHLYKQISNGTSGGSSNDNTYFGYSTPWGYFDFNRFHCHFSPRDWQRLINNN |
| | WGFRPKRLNFKLFNIQVKEVTQNEGTKTIANNLTSTVQVFTDSKYQLPYVLGSAHQGCLP |
| | PFPADVFMIPQYGYLTLNNGSQAVGRSSFYCLEYFPSQMLRTGNNFTFSYTFEDVPFHSS |
| | YAHSQSLDRLMNPLIDQYLYYLSRTQTNGTNATQTLLFAQAGPQNMSAQAKNWLPGPCYR |
| | QQRVSTTVSQNNNSNFTWTGATKYHLNGRDSLVNPGVAMATHKDDEERFFPSSGVLMFGK |
| | QGAGKDNVEYTNVMLTSEEEIKTTNPVATEQYGVVADNLQQTNSAPIVGAVNSQGALPGM |
| | VWQNRDVYLQGPIWAKIPHTDGNFHPSPLMGGFGLKHPPPQILIKNTPVPADPPVNFTDA |
| | KLASFITQYSTGQVSVEIEWELQKENSKRWNPEIQYTSNYYKSANVDFAVNADGVYSEPR |
| | PIGTRYLTRNL |
| 1380 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANSLTSTIQVFTDSEYQLPYVLGSA |
| | HQGCLPPFPADVFMIPQYGYLTPNNGSQAVGRSSFYCLEYFPSQMLRTGNNFSFSYTFED |
| | VPFHSSYAHSQSLDRLMNPLIDQYLYYLSRTQSTGGTAGTQQLLSSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWELQKENSKRWNPEIQYTSNYYKSTNVDFAVNTE |
| | GVYSEPRPIGTRYLTRNL |
| 1381 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYVHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNSGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWELQKENSKRWNPEIQYTSNYYKSTNVDFAVNTE |
| | GVYSEPRPIGTRYPTRNL |
| 1382 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPAYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLSGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSSITQYSTGQVSVEIEWELQKENSKRWNPEIQYTSNYYKSTNVDFAVNTE |
| | GVYSEPRPIGTRYLTRNL |
| 1383 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIVWELQKENSKRRNPEIQYTSNYYKSTNVDFAVNTE |
| | GVYSEPRPIGTRYLTRNL |
| 1384 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPPS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVVIEWELQKENSKRWNPEIQYTSNYYKSTNVDFAVNTE |
| | GVYSELRPIGTRYLTRNL |
| 1385 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKATNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWELQKENSKRWNPEIQYTSNYYKSTNVDFAVNTE |
| | GVYSEPRPIGTRYLTRNL |
| 1386 | MAPGKKRPVEPSPQRSPDSSTGIGKKGQQPARKRLNFGRTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYLDFNRFHCHFSPRDWQ |
| | RLINNNWGFRPKRLSFKLFNIQVKEVTQNEGTKTIANNLASTIQVFTDSEYQPPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSRAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPTVGAVNS |
| | QGALPGMVWQNRDVYLQGPIWAKIPHTDGNFHPSPLMGGFGLKHPPPQILIKNTPVPADP |
| | PTAFNQAKLNSFIAQYSTGQVSVEIEWELQKENSKRWNPEIQYTSNYYKSTNADFAVNTE |
| | GVYSEPRPIGTRYLTRNL |
| 1387 | MAPGKKRPVEPSPQRSPDSSTGIGKKGQQPARKRLNFGQTGDS |
| | ESVPDPQLIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGNLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNMGYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYGTGQVSVEIEWELQKENSKRWNPEIQYTSNYYKSTNVDFAVNTE |
| | GVYSEPRPIGTRYLTRNL |
| 1388 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCQPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWEPQKENSKRWNPEIQYTSNYYKSTNVDFAVNTE |
| | GVYSEPRPIGTRYLTRNL |
| 1389 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQTSNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSAEIEWELQKENSKRWNPEIQYTSNYYKSTNVDFAVNTE |
| | GVYSEPRPIGTRYLTRNL |
| 1390 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKSTPVPADP |
| | PTAFNQAKLNSFITQYSTGQVSVEIEWELQKENSKCWNPEIQYTSNYYKSTNVDFAVNTE |
| | GVYSEPRPIGTRYLTRNL |
| 1391 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RPINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYPFED |
| | VPFHSSYAHSQSLDRLMNPLIDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQDTAPIVGAVNS |
| | QGALPGMVWQNRDVYLQGPIWAKIPHTDGNFHPSPLMGGFGLKHPPPQVLIKNTPVPADP |
| | PTAFNQAKLNSFITQYSTGQVSVEIEWELQKENSKRWNPEIQYTSNYYKSTNVDFAVNTE |
| | GVYSEPRPIGTRYLTRNL |
| 1392 | MAPGKKRPVEPSPQRSPDSSTGIGKKGQQPAGKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLVDQYLYYLSRTQSTGGTAGTQQLLFSQAGPSNMSAQARNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWELQKENSKRWSPEIQYTSNYYKSTNVDFAVNTE |
| | GVYSEPRPIGTRYLTRNL |
| 1393 | MAPGKKRPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQAVGRSSFYCLEYFPSQMLRTGNNFSFSYTFEDV |
| | PFHSSYAHSQSLDRLMNPLIDQYLYYLSRTQSTGGTAGTQQLLFSQAGPSNMSTQARNWL |
| | PGPCYRQQRVSTTLSQNNNSNFAWTGATKYHLNGRDSLVNPGVAMATNKDDEDRFFPSSG |
| | ILMFGKQGAGKDNVDYSNVMLTSEEEIKTTNPVATEQYGVVADNLQQQNTAPIVGAVNSQ |
| | GALPGMVWQNRDVYLQGPIWAKIPHTDGNFHPSPLMGGFGLKHPPPQILIKNTPVPADPP |
| | TAFNQAKLNSFITQYSTGQVSVEIEWELQKENSKRWNPEIQYTSNYYKSTNVDFAVNTEG |
| | AYSEPRPIGTRYLTRN |
| 1394 | MAPGKKRPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNPTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCYRQQRVSTTLSQNNNSNFAWTGATKYHLNGRDSLVNPGVAMATNKDDEDRFFPSS |
| | GILMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTAFNQAKLNSFITQYSTGQVSVEIEWELQKENSKRWNPEIQYTSNYYKSTNVDFAVNTE |
| | GVYSEPRPIGTRYLTRNL |
| 1395 | MAPGKKGPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDEQTGVDFAVDSQG |
| | VYSEPRPIGTRYLTRNL |
| 1396 | MAPGKKRPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPRPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1397 | MAPGKKRPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1398 | MAPGKKRPVEPSPQRSPDSSTGIGKKGQQPARKRLNFGQTGDS |
| | ESVPDPQPIGEPPAGPSGLGSGTMAAGGGAPMADNNKGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTGDGVTTIANNLTSTVQVFSDSEYQLPYVLGSAH |
| | QGCLPPFPADVFMIPQYGYLTLNNDSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1399 | MAPGKKRPVEPSPQRSPDSSTGIGKKGQQPARKRLNFGQTGDS |
| | ESVPDPQPIGEPPAGPSGLGSGTMAAGGGAPMADNNKGADGVGNASGNWHCDSTWLGDRV |
| | ITTSTRTWALPTYNNHLYKQISSQSAGSTNDNVYFGYSTPWGYFDFNRFHCHFSPRDWQR |
| | LINSNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1400 | MAPGKKRPVEPSPQRSPDSSTGIGKKGQQPARKRLNFGQTGDS |
| | ESVPDPQPLGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQVLRTGNNFTFSYTFEDV |
| | PFHSSYAHSQSLDRLMNPLIDQYLYYLARTQSNPGGTSGNRELQFYQGGPSTMAEQAKNW |
| | LPGPCFRQQRVSKTLDQNNNSNFAWTGATKYHLNGRNSLVNPGVAMATHKDDEDRFFPSS |
| | GVLIFGKTGATNKTTLENVLMTNEEEIRPTNPVATEEYGIVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1401 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTRLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTFSYTFEDV |
| | PFHSSYAHSQSLDRLMNPLIDQYLYYLARTQSNAGGTAGNRELQFYQGGPTTMAEQAKNW |
| | LPGPCFRQRRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNFDKQTGVDFAVDSQG |
| | VYSEPRPIGTRYLTRNL |
| 1402 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTVQVFSDSEYQLPYVLGSA |
| | HQGCLPPFPADVFMIPQYGYLTLNNGSQSVGRSSFYCLEYFPSQMLRTGNNFTFSYTFED |
| | VPFHSSYAHSQSLDRLMNPLIDQYLYYLARTQSNAGGTAGNRELQFYQGGPTTMAEQAKN |
| | WLPGPCFRQQRVSKTLDQNNNSNFAWTGATKYHLNGRNSLVNPGVAMATHKDDEERFFPS |
| | SGVLIFGKTGAANKTTLENVLMTNEEEIRPTNPVATEEYGTVSSNLQAANTAAQTQVVNN |
| | QGALPGMVWQNRDVYLQGPIWAKIPHTDGNFHPSPLMGGFGLKHPPPQILIKNTPVPANP |
| | PEVFTPAKFASFITQYSTGQVSVEIEWELQKENSKRWNPEIQYTSNFDKQTGVDFAVDSQ |
| | GVYSEPRPIGTRYLTRNL |
| 1403 | MAPGKKRPVEPSPQRSPDSSTGIGKKGQQPARKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFTISYTFEDV |
| | PFHSSYAHSQSLDRLMNPLIDQYLYYLARTQSNAGGTAGNQELQFYQGGPTTMAEQAKNW |
| | LPGPCFRQQRVSKTLDQNNNSNFAWTGATKYHLNGRNSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILIKNTPVPADPP |
| | TAFNQAKLNSFITQYSTGQVSVEIEWELQKENSKRWNPEIQYTSNYYKSTNVDFAVNTEG |
| | VYSEPRPIGTRYLTRNL |
| 1404 | MAPGKKRPVEPSPQRSPDSSTGIGKKGQQPARKRLNFGQTGDS |
| | ESVPDPQPIGEPPAAPSSVGSGTMAAGGGAPMADNNEGADGVGSSSGNWHCDSTWLGDRV |
| | ITTSTRTWALPTHNNHLYKQISNGTSGGSTNDNVYFGYSTPWGYFDFNRFHCHFSPRDRQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFSFSYTFED |
| | VPFHSSYAHSQSLDRLMNPLIDQYLYYLSRTQSTEGTAGTQQLLFSQAGPSNMSAQARNW |
| | LPGPCYRQQRVSTTLSQNNNSNFAWTGATKYHLNGRDSLVNPGVAMATHKDDEERFFPSS |
| | GVLIFGKTGAANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPQILIKNTPVPANPPEV |
| | FTPAKFASFITQYSTGQVSVEIEWELQKENSKRWNPEIQYTSNFDKQTGVDFAVDSQGVY |
| | SEPRPIGTRYLTRNL |
| 1405 | MAPGKKRPVEQSPQEPDSSAGIGKSGSQPAKKKLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGGQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWPGASSWALNGRNSLMNPGPAMASHKEGEDRFFPLSGS |
| | LIFGKQGTGRDNVDADKVMITNEEEIKTTNPVATESYGQVATNHQSAQAQAQTGWVQNQG |
| | ILPGMVWQDRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVEFAVSTEGV |
| | YSEPRPIGTRYLTRNL |
| 1406 | MAPGKKRPVEQSPQEPDSSAGIGKSGSQPAKKKLNFGQTGDTE |
| | SVPDPQPIGEPPAAPSGVGSLTMASGGGAPVADNNEGADGVGSSSGNWHCDSQWLGDRVI |
| | TTSTRTWALPTYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDYQLPYVLGSAH |
| | EGCLPPFPADVFMIPQYGYLTLNDGSQAVGRSSFYCLEYFPSQMLRTGNNFQFSYEFENV |
| | PFHSSYAHSQSLDRLMNPLIDQYLYYLSKTINGSGQNQQTLKFSVAGPSNMAVQGRNYIP |
| | GPSYRQQRVSTTVTQNNNSEFAWPGASSWALNGRNSLMNPGPAMASHKEGEDRFFPLSGS |
| | LIFGKQGTGRDNVDADKVMITNEEEIKTTNPVATESYGQVATNHQSAQAQAQTGWVQNQG |
| | ILPGMVWQDRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVEFAVNTEGV |
| | YSEPRPIGTRYLTRNL |
| 1407 | MAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTGTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLGRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1408 | MAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNESQAVGRSSFYCLEYFPSQMLRTGNNFTFSYTFEDVPF |
| | HSSYAHSQSLGRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1409 | MAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAAGRSSFYCLEYFPSQMLRTGNNFTFSYTFEDVPF |
| | HSSYAHSQSLGRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1410 | MAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSGYQLPYVLGSAHQG |
| | CLPPFPADVFMVPQYGYPTLNNGSQAVGRSSFYCLEYFPSQMLRTGNNFTFSYTFEDVPF |
| | HSSYAHSQSLDRLMNPLIDQYLYYLSTTNTPSGTTTQSRLQFSQAGASDIRDQSRNWLPG |
| | PCYRQQRVSKTSADNNNSEYSWTGATKYHLNGRDSLVNPGPAVASHKDDEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1411 | MAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDSHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSTTNTPSGTTTQSRLQFSQAGASDIRDQSRNWLPG |
| | PCYRQQRVSKTSADNNNSEYSWTGATKYHLNGRDSLVNPGPAMASHKDNEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1412 | MAPGKKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQGGNTQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1413 | MAPGKKRPVEHSPAEPDSSSGTGKSGNQPARKRLNFGQTGDSD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIQDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQSGNTQAATADVNTQGV |
| | LPGMVGQDRDVYLQGPTWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1414 | MAPGKKRPVEHSPAEPDSSSGTGKSGNQPARKRLNFGQTGDSD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNSFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQSGNTQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1415 | MAPGKKRPVEHSPAEPDSSSGTGKSGNQPARKRLNFGQTGDSD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLGYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGPEKTNVDIEKVMITDEEEIRTTNPVATEQYGSVSTNLQSGNTQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1416 | MAPGEKRPVEHSPAEPDSSSGTGKAGQQPARKRLNFGQTGDAD |
| | SVPDPQPLGQPPAAPSGLGTNTMASGSGAPMADNNEGADGVGNSSGNWYCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLVGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1417 | MAPGEKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGHFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPPMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGARYLTRNL |
| 1418 | MAPGKKRPVEHSPAEPDSSSGTGKAGQQPARKRLNFGQTGDAD |
| | SVPDPRPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1419 | MAPGKKRPVEHSPAEPDSSSGTGKAGQQPARKRLNFGQTGDAD |
| | SVPDPRPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1420 | MAPGKKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGDRVI |
| | TTSTRTWALPTCNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTYLQSGNTQAATSDVNTQGV |
| | LPGMVWQDRDVYLRGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1421 | MAPGKKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGGRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQTLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1422 | MAPGKKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1423 | MAPGKKRPVEHSPAEPDSSSGTGKAGQQPARKRLNFGQTGDAD |
| | SVPDPQPLGQPPAAPSGLGTNTMASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSGYQLPYVLGSAHQG |
| | CLPPFPADVFMVPQYGYLTLNNGSQAVGRSSFYCLEYFPSQMLRTGNNFTFSYTFEDVPF |
| | HSSYAHGQSLDRLMNPLIDQYLYYLSRTNTPSGTTTMSRLQFSQAGASDVRDQSRNWLPG |
| | PCYRQQRVSKTAADNNNSDYSWTGATKYHLNGRDSLVNPGPAMASHKDDEEKYFPQSGVL |
| | VFGKQDSGKTNVDIEKVMITDEEEIRTTNPAATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFVSFITQYSTGQVSVEIEWELQKENSKRWNPEIQYTSNYNKSVNVDFTVDINGVY |
| | SEPRPIGTRYLTRNL |
| 1424 | MAPGKKRPVEHSPVEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLV |
| | NNNRGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQTNSGTLQQSRLLFSQAGPTNMSLQAKNWLPG |
| | PCYRQQRLSKQANDNNNSNFPWTAATKYHLNGRDSLVNPGPAMASHKDDEEKFFPMHGTL |
| | IFGKQGTNANDADLENVMITDEEEIRATNPVATEQYGTVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1425 | MAPGKKRPVEHSPVEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQTNSGTLQQSRLLFSQAGPTNMSLQAKNWLPG |
| | PCYRQQRLSKQANDNNNSNFPWTAATKYHLNGRDSLVNPGPAMASRKDDEEKFFPMHGTL |
| | IFGKQGTNANDADLENVMITDEEEIRATNPVATEQYGTVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1426 | MAPGKKRPVEHSPVEPDSSSGTGKAGQRPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQTNSGTLQQSRLLFSQAGPTNMSLQAKNWLPG |
| | PCYRQQRLSKQANDNNNSNFPWTAATKYHLNGRDSLVNPGPAMASHKDDEEKFFPMHGTL |
| | IFGKQGTNANDADLENVMITDEEEIRATNPVATEQYGTVSNNLQNSNTGPTTGTVNRQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1427 | MAPGKKRPVEHSPVEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNGGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQTNSGTLQQSRLLFSQAGPTNMSLQAKNWLPG |
| | PCYRQQRLSKQANGNNNSNFPWTAATKYHLNGRDSLVNPGPAMASHKDDEEKFFPMHGTL |
| | IFGKQGTNANDADLENVMITDEEEIRATNPVATEQYGTVSNNLQNSNTGPTTGTVNHQGA |
| | LPGMVWQDRDVYLQGPIWAKIPHTDGHFHPSPLTGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1428 | MAPGKKRPVEHSPVEPDSSSGTGKAGQQPARKRLNFGQTGDA |
| | DSVPDPQPLGQPPAAPSGLGSTTMATGSGAPMADNNEGADGVGNSSGNWHCDSQWLDDRV |
| | IATSTRTWALPTYNNHLYKQISSQSGACNDNHYFGYSTPWGYFDFNRFHCHFSPRDWQRL |
| | INSNWGFRPKRLNFKLFNIQVKEVTQNDGTTTIANNLTSTVQVFTDSEYQLPYVPGSAHQ |
| | GCLPPFPADVFMVPQYGYLTLNNGSQAVGRSSFYCLEYFPSQMLRTGNNFQFSYTFEDVP |
| | FHSSYAHCQSLDRLMNPLIDQYLYYLNKTQTNSGTLQQSRLLFSQAGPTNMSLQAKNWLP |
| | GPCYRQQRLSKQANDNNNCNFPWTAATKYHLNGRDSLVNPGPAMASHKDDEEKFFPMHGT |
| | LIFGKQGTNANDADLENVMITDEEEIRPTNPVATEQYGTVSNNLQNSNTGPTTGTVNHQG |
| | ALPGMVWQDRDVYLQGPIWAKIPHTDGHFHPSPLMGGFGLKHPPPQIMIKSTPVPANPPT |
| | NFSSAKFASSITQYSTGQVSVEIEWELQKENSKRWNPEIQYTSNYNKSVNVDFTVDTNGV |
| | YSEPRPIGTRYLTRNL |
| 1429 | MAPGKKRPVEHSPAEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSPFYCLEYFPSQMLRTGNNFQFSYTFEDVPF |
| | HSSYAHSQSLDRLMNPLIDQYLYYLNKTQTNSGTLQQSRLLFSQAGPTNMSLQAKNWLPG |
| | PCYRQQRLSKQANDNNNSNFPWTAATKYHLNGRDSLVNPGPAMASHKDDEEKFFPMHGTL |
| | IFGKQGTNANDADLENVMITDEEEIRTTNPVATEQYGTVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDNNGVY |
| | SEPRPIGTRYLTRNL |
| 1430 | MAPGKKRPVEHSPVEPDSSSGTGKAGNQPARKRLNFGQTGDAD |
| | SVPDPQPLGQPPAAPSGLGSTTMATGSGAPVADNNEGADGVGNSSGNWHCDSQWLGDRVI |
| | TTSTRTWALPTYNNHLYKQISSQSGASNDNHYFGYSTPWGYFDFNRFHCHFSPRDRQRLI |
| | NNNWGFRPKRLNFKLFNIQVKEVTQNDGTTTIANNLTSTVQVFTDSGYQLPYVLGLAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKEDSKRWNPEIQYTSNYNKPVNVDFTVDINGVY |
| | SEPRPIGTRYLTRNL |
| 1431 | MAPGKKRPVEHSPVEPDSSSGTGKAGNQPARKRLNFGQTGDAD |
| | SVPDPQPLGQPPAAPSGLGSTTMATGSGAPVADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQDSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1432 | MAPGKKRPVEHSPVEPDSSSGTGKAGQQPARKRLNFGQTGDAD |
| | SVPDPQPLGQPPAAPSGLGSTTMASGSGAPVADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNSWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLLNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSSKFASFITQYSTGQVSVEIEWELQKENSKRWNPEIQYTSNYNKSVNVDFTVDTNGVY |
| | SEPRPIGTRYPTRNL |
| 1433 | MAPGKKRPVEHSPAEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1434 | MAPGKKRPVEHSPAEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1435 | MAPGKKRPVEHSPAEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWAQPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQSNSGALQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHRGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1436 | MAPGKKRPVEQSPQEPDSSAGIGKSGAQPAKKRLNFGQTGDTESVPDPQPIGEPPAAPSG |
| | VGSLTMASGGGAPVADNNEGADGVGSSSGNWHCDSQWLGDRVITTSTRTWALPTYNNHLY |
| | KQISNSTSGGSSNDNAYFGYSTPWGYFDFNRFHCHFSPRDWQRLINNNWGFRPKRLNFKL |
| | FNIQVKEVTDNNGVKTIANNLTSTVQVFTDSDYQLPYVLGSAHEGCLPPFPADVFMIPQY |
| | GYLTLNDGSQAVGRSSFYCLEYFPSQMLRTGNNFQFSYEFENVPFHSSYAHSQSLDRLMN |
| | PLIDQYLYYLSKTINGSGQNQQTLKFSVAGPSNMAVQGRNYIPGPSYRQQRVSTTVTQNN |
| | NSEFAWPGASSWALNGRNSLMNPGPAMASHKEGEDRFFPLSGSLIFGKQGTGRDNVDADK |
| | VMITNEEEIKTTNPVATESYGQVATNHQSNSRGDYNSLAQAQTGWVQNQGILPGMVWQDR |
| | DVYLQGPIWAKIPHTDGNFHPSPLMGGFGMKHPPPQILIKNTPVPADPPTAFNKDKLNSF |
| | ITQYSTGQVSVEIEWELQKENSKRWNPEIQYTSNYYKSNNVEFAVNTEGVYSEPRPIGTR |
| | YLTRNL |
| 1437 | MAPGKKRPVEQSPQEPDSSAGIGKSGAQPAKKRLNFGQTGDTESVPDPQPIGEPPAAPSGV |
| | GSLTMASGGGAPVADNNEGADGVGSSSGNWHCDSQWLGDRVITTSTRTWALPTYNNHLYK |
| | QISNSTSGGSSNDNAYFGYSTPWGYFDFNRFHCHFSPRDWQRLINNNWGFRPKRLNFKLF |
| | NIQVKEVTDNNGVKTIANNLTSTVQVFTDSDYQLPYVLGSAHEGCLPPFPADVFMIPQYG |
| | YLTLNDGSQAVGRSSFYCLEYFPSQMLRTGNNFQFSYEFENVPFHSSYAHSQSLDRLMNP |
| | LIDQYLYYLSKTINGSGQNQQTLKFSVAGPSNMAVQGRNYIPGPSYRQQRVSTTVTQNNN |
| | SEFAWPGASSWALNGRNSLMNPGPAMASHKEGEDRFFPLSGSLIFGKQGTGRDNVDADKV |
| | MITNEEEIKTTNPVATESYGQVATNHQSGQSGRGDAVGVAQAATGWVQNQGILPGMVWQD |
| | RDVYLQGPIWAKIPHTDGNFHPSPLMGGFGMKHPPPQILIKNTPVPADPPTAFNKDKLNS |
| | FITQYSTGQVSVEIEWELQKENSKRWNPEIQYTSNYYKSNNVEFAVNTEGVYSEPRPIGT |
| | RYLTRNL |
| 1438 | MAPGKKRPVEQSPQEPDSSAGIGKSGAQPAKKRLNFGQTGDTESVPDPQPIGEPPAAPSG |
| | VGSLTMASGGGAPVADNNEGADGVGSSSGNWHCDSQWLGDRVITTSTRTWALPTYNNHLY |
| | KQISNSTSGGSSNDNAYFGYSTPWGYFDFNRFHCHFSPRDWQRLINNNWGFRPKRLNFKL |
| | FNIQVKEVTDNNGVKTIANNLTSTVQVFTDSDYQLPYVLGSAHEGCLPPFPADVFMIPQY |
| | GYLTLNDGSQAVGRSSFYCLEYFPSQMLRTGNNFQFSYEFENVPFHSSYAHSQSLDRLMN |
| | PLIDQYLYYLSKTINGSGQNQQTLKFSVAGPSNMAVQGRNYIPGPSYRQQRVSTTVTQNN |
| | NSEFAWAAASSWALNGRNSLMNPGPAMASHKEGEDRFFPLSGSLIFGKQGTGRDNVDADK |
| | VMITNEEEIKTTNPVATESYGQVATNHQSGQSGRGDLGLSAQAATGWVQNQGILPGMVWQ |
| | DRDVYLQGPIWAKIPHTDGNFHPSPLMGGFGMKHPPPQILIKNTPVPADPPTAFNKDKLN |
| | SFITQYSTGQVSVEIEWELQKENSKRWNPEIQYTSNYYKSNNVEFAVNTEGVYSEPRPIG |
| | TRYLTRNL |
| 1439 | MAPGKKRPVEHSPVEPDSSSGTGKAGHQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLNRTQSNSGTLQQSRLLFSQAGPTSMSLQAKNWLPG |
| | PCYRQQRLSKQANDNNNSNFPWTAATKYRLNGRDSLVNPGPAMASHKDDEEKFFPMHGTL |
| | IFGKQGTNANDADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1440 | MAPGKKRPVEHSPVEPDSSSGTGKAGHQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRPSFYCLEYFPSQMLRTGNNFTFSYTFEDVPF |
| | HSSYAHSQSLDRLMNPLIDQYLYYLNRTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1441 | MAPGKKRPVEHSPVEPDSSSGTGKAGHQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRPSFYCLEYFPSQMLRTGNNFTFSYTFEDVPF |
| | HSSYAHSQSLDRLMNPLIDQYLYYLNRTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDINGVY |
| | SEPRPIGTRYLTRNL |
| 1442 | MAPGKKRPVEHSPVEPDSSSGTGKAGHQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFTVPQYGYLTLNNGSQAVGRSSFYCLEYFPSQMLRTGNNLTFSYTFEDVPF |
| | HSSYAHSQSLDRLMNPLIDQYLYYLNRTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKNTPVPANPPTN |
| | YSSAKFASFITQYSTGQVSVEIEWELRKENSKRWNPEIQYTSNYNKSVNVDFTVDINGVY |
| | SEPRPIGTRYLTRNL |
| 1443 | MAPGKKRPVEQSPQEPDSSSGIGKTGQQPAKKRLNFGQTGDSE |
| | SVPDPQPLGEPPATPAAVGPTTMASGGGAPMADNNEGADGVGNASGNWHCDSTWLGDRVI |
| | TTSTRTWALPTYNNHLYKQISSTSTGASNDNHYFGYGTPWGYFDFNRFHCHFSPRDWQRL |
| | INNNWGFRPKRLNFKLFNIQVEEVTTNDGVTTIANNLTSTVQVFSDSEYQLPYVLGSAHQ |
| | GCLPPFPADVFMIPQYGYLTLNNGSQAVGRSSFYCLEYFPSQMLRTGNNFTFSYTFEEVP |
| | FHSSYAHSQSLDRLMNPLIDQYLYYLNRTQNQSGSAQNKDLLFSRGSPAGMSVQPKNWLP |
| | GPCYRQQRVSKTKTDNNNSNFTWTGASKYNLNGRESIINPGTAVASHKDDEDKFFPMSGV |
| | MIFGKESAGASSTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIKNTPVPANPPA |
| | EFSATKFASFITQYSTGQVSVEIEWELQKENSKRWNPEVQYTSNYAKSANVDFTVDNNGL |
| | YTEPRPIGTRYLTRPL |
| 1444 | MAPGKKRPVEQSPQGPDSSSGIGKTGQQPAKKRLNFGQTGDSE |
| | SVPDPQPLGEPPATPAAVGPTTMASGGGAPMADNNEGADGVGNASGNWHCDSTWLGDRVI |
| | TTSTRTWALPTYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQLPYVLGSAHQ |
| | GCLPPFPADVFMIPQYGYLTLNNGSQAVGRSSFYCLEYFPSQMLRTGNNFTFSYTFEEVP |
| | FHSSYAHSQSLDRLMNPLIDQYLYYPNRTQNQSGSAQNKDLLFSRGSPAGMSVQPKNWLP |
| | GPCYRQQRVSKTKTDNNNSNFTWTGASKYNLNGRESIINPGTAMASHKDDEDKFFPMSGV |
| | MIFGKESAGASNTALDNVMITDEEEIKATNPVATERFGTVAVNFQSSSTDPATGDVHAMG |
| | ALPGMVWQGRDVYLQGPIWAKIPHTDGHFHPSPLMGGFGLKNPPPQILIKNTPVPANPPA |
| | EFSATKFASFITQYSTGQVSVEIEWELQKENSKRWNPEVQYTSNYAKSANVDFTVDNNGL |
| | YTEPRPIGTRYLTRPL |
| 1445 | MAPGKKRPVEQSPQEPDSPSGIGKTGQQPAKKRLNFGQTGDSE |
| | SVPDPQPLGEPPATPAAVGPTTMASGGGAPMADNNEGADGVGNASGNWHCDSTWLGDRVI |
| | TTSTRTWALPTYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQLPYVLGSAHQ |
| | GRLPPFPADVFMIPQYGYLTLNNGSQAVGRSSSYCLEYFPSQMLRTGNNFTFSYTFEEVP |
| | LHSSCAHSQSLDRLMNPLIDQYLYYLNRTQNQSGSAQNRDLLFSRGSPAGMSVQPKNWLP |
| | GPCYRQQRVSKTKTDNNNSNFTWTGASKYNLNGRESIINPGTAMASHKDDEDKFFPMSGV |
| | MIFGKESAGASNTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIKNTPVPANPPA |
| | EFSATKFASFITQYSAGQVSVEIEWELQKENSKRWNPEVQYTSNYAKSANVDFTVDNNGL |
| | YTEPRPIGTRYLTRPL |
| 1446 | MAPGKKRPVEPSPQRSPDSSTGIGKKGQQPAKKRLNFGQTGDS |
| | ESVPDPQPIGEPPAGPSGLGSGTMAAGGGAPMADNNEGADGVGNASGNWHCDSTWLGDRV |
| | ITTSTRTWALPTYNNHLYKQISSASTGASNDNHYFGYSTPWGYFDFNRFHCHFSPRDWQR |
| | LINNNWGFRPKRLNFKLFNIQVKEVTTNDGVTTIANNLTSTVQVFSDSEYQLPYVLGSAH |
| | QGCLPPFPADVFMIPQYGYLTLNNGSQAVGRSSFYCLEYFPSQMLRTGNNFTFSYTFEEV |
| | PLHSSYAHSQSLDRLMNPLIVQYLYYLNRTQNQSGSAQNKDLLFSRGSPAGMSVQPKNWL |
| | PGPCYRQQRVSKTKTDNNNSNFTWTGASKYNLNGRESIINPGTAMASHKDDEDKFFPMSG |
| | VMIFGKESAGASNTALDNVMITDEEEIKATNPVATERFGTVAVNFQSSSTDPATGDVHAM |
| | GALPGMVWQDRDVYLQGPIWAKIPHTDGHFHPSPLMGGFGLKNPPPQILIKNTPVPANPP |
| | AEFSATKFASFITQYSTGQVSVEIEWELQKENSKRWNPEVQYTSNYAKSASVDFTVDNNG |
| | LYTEPRPIGTRYLTRPL |
| 1447 | MAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNCHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1448 | MAPGKKRPVEQSPQEPDSSSGIGKTGQQPAKKRLNFGQTGDSE |
| | SVPDPQPLGEPPATPAAVGPTTMASGGGAPMADNNEGADGVGNASGNWHCDSTWLGDRVI |
| | TTSTRTWALPTYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQLPYVLGSAHQ |
| | GCLPPFPADVFMIPQYGYLTLNNGSQAVGRSSFYCLEYFPSQMLRTGNNFTFSYTFEEVP |
| | FHSSYAHSQSLDRLMNPLIDQYLYYLNRTQNQSGSAQNKDLLFSRGSPAGMSVQPKNWLP |
| | GPCYRQQRVSKTKTDNNNSNFTWTGASKYNLNGRESIINPGTAMASHKDDEDKFFPMSGV |
| | MIFGKESAGASNTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIKNTPVPANPPA |
| | EFSATKFASFITQYSTGQVSVEIEWELQKENSKRWNPEVQYTSNYAKSANVDFTVDNNGL |
| | YTEPRPIGTRYLTRPL |
| 1449 | MAPGKKRPVEQSPQEPDSSSGIGKKGQQPARKRLNFGQTGDSE |
| | SVPDPQPLGEPPAAPSGVGPNTMAAGGGAPMADNNEGADGVGSSSGNWHCDSTWLGDRVI |
| | TTSTRTWALPTYNNHLYKQISNGTSGGATNDNTYFGYSTPWGYFDFNRFHCHFSPRDWQR |
| | LINNNWGFRPKRLSFKLFNIQVKEVTQNEGTKTIANNLTSTIQVFTDSEYQLPYVLGSAH |
| | QGCLPPFPADVFMIPQYGYLTLNNGSQAVGRSSFYCLEYFPSQMLRTGNNFQFTYTFEDV |
| | PFHSSYAHSQSLDRLMNPLIDQYLYYLSRTQTTGGTANTQTLGFSQGGPNTMANQAKNWL |
| | PGPCYRQQRVSTTTGQNNNSNFAWTAGTKYHLNGRNSLANPGIAMATHKDDEERFFPVTG |
| | SCFWQQNAARDNADYSDVMLTSEEEIKTTNPVATEEYGIVADNLQQQNTAPQIGTVNSQG |
| | ALPGMVWQNRDVYLQGPIWAKIPHTDGNFHPSPLMGGFGLKHPPPQILIKNTPVPADPPT |
| | TFNQSKLNSFITQYSTGQVSVEIEWELQKENSKRWNPEIQYTSNYYKSTSVDFAVNTEGV |
| | YSEPRPIGTRYLTRNL |
| 1450 | MAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLGRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1451 | MAPGKKRPVEHSPVEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLVDQYLYYLNKTQTNSGTLQQSRLLFSQAGPTNMSLQAKNWLPG |
| | PCYRQQRLSKQANDNNNSNFPWTAATKYHLNGRDSLVNPGPAMASHKDDEEKFFPMHGTL |
| | IFGKQGTNANDADLENVMITDEEEIRTTNPVATEQYGTVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1452 | MAPGKKRPVEHPPVEPDSSSGTGKAGQQPARKRLNFGQTGDAD |
| | SVPDPQPLGQPPAAPSGLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNKTQTNSGTLQQSRLLFSQAGPTNMSLQAKNRLPG |
| | PCYRQQRLSKQANDNNNSNFPWTAATKYHLNGRDSLVNPGPAMASHKDDEEKFFPMHGTL |
| | IFGKQGTNANDADLENVMITDEEEIRTTNPVATEQYGTVSNNLQNSNTGPTTGTVNHQGA |
| | LPGMVWQDRDVYLQGPIWAKIPHTDGHFHPSPLVGGFGLKHPPPQIMIKNTPVPANPPTN |
| | FSSAKFASFITQYSTGQVSVEIEWELQKENSKRWNPEIQYTSNYNKSVNVDFTVDTNGVY |
| | SEPRPIGTRYLTRNL |
| 1453 | MAPGKKRPVEHSPAEPDSSSGTGKAGQQPARKRLNFGQTGDAD |
| | SVPDPQPLRQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLMNPLIDQYLYYLNRTQTASGTQQSRLLFSQAGPTSMSLQAKNWLPGP |
| | CYRQQRLSKQANDNNNSNFPWTGATKYYLNGRDSLVNPGPAMASHKDDEEKFFPMHGTLI |
| | FGKEGTNATNAELENVMITDEEEIRTTNPVATEQYGYVSNNLQNSNTAASTETVNHQGAL |
| | PGMVWQDRDVYLQGPIWAKIPHTDGHFHPSPLMGGFGLKHPPPQIMIKNTPVPANPPTNF |
| | SSAKFASFITQYSTGQVSVEIEWELQKENSKRWNPEIQYTSNYNKSVNVDFTVDINGVYS |
| | EPRPIGTRYLTRNL |
| 1454 | MAPGKKRPVEHSPAEPDSSSGTGKAGQQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLECFPSQMLRTGNNFQFSYTFEDVPF |
| | HSSYAHSQSLDRLMNPLIDQYLYYLNRTQTASGTQQSRLLFSQAGPTSMSLQAKNWLPGP |
| | CYRQQRLSKQANDNNNSNFPWTGATKYHLNGRDSLVNPGPAMASHKDDEEKFFPMHGTLI |
| | FGKEGTNATNAELENVMITDEEEIRTTNPVATEQYGYVSNNLQNSNTAASTETVNHQGAL |
| | PGMVWQDRDVYLQGPIWAKIPHTDGHFHPSPLMGGFGLKHPPPQIMIKNTPVPANPPTNF |
| | SSAKFASFITQYSTGQVSVEIEWELQKENSKRWNPEIQYTSNYNKSVNVDFTVDTNGVYS |
| | EPRPIGTRYLTRNL |
| 1455 | MAPGKKRPVEHSPAEPDSSSGTGKAGQQPARKRLNFGQTGDAD |
| | SVPDPQPLGQPPAAPTSLGSTTMATGSGAPMADNNEGADGVGNSSGNWHCDSQWLGDRVI |
| | TTSTRTWALPTYNNHLYKQISSQSGASNDNHYFGYSTPWGYFDFNRFHCRFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQGLDRLMNPLIDQYLYYLNRTQTASGTQQSRLLFSQAGPTSMSLQAKNWLPGP |
| | CYRQQRLSKQANDNNNSNFPWTGATKYHLNGGDSLVNPGPAMASHKDDEEKFFPMHGTLI |
| | FGKEGTNATNAELENVMITDEEEIRTTNPVATEQYGYVSNNLQNSNTAASTETVNHQGAL |
| | PGMVWQDRDVYLRGPIWAKIPHADGHFHPSPLMGGFGLKHPPPQIMIKNTPVPANPPTNF |
| | SSAKFASFITQYSTGQVSVEIEWELQKENSKRWNPEIQYTSNYNKSVNVDFTVDTNGVYS |
| | EPRPIGTRYLTRNL |
| 1456 | MAPGKKRPVEHSPVEPDSSSGTGKAGQQPARKRLNFGQTGDAD |
| | SVPDPQPLGQPPAAPSGLGTNTMATGSGAPMADNNEGADGVGNSSGSWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVPGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTAADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQSGNTQAATADVNTQGV |
| | LPGMVWQDRDVHLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1457 | MAPGKKRPVEHSPVEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSGYAHSQSLDRLMNPLIDQYLYYLSTTNTPSGTTTQSRLQFSQAGASDIRDQSRNWLPG |
| | PCYRQQRVSKTSADNNNSEYSWTGATKYHLNGRDSLVNPGPAMASHKDNEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1458 | MAPGKKRPVEHSPVEPDSSSGTGKAGQQPARKRLNFGQTGDAD |
| | SVPDPQPLGQPPAAPSGLGTNTMATGSGAPMADNNEGADGVGNSSGNRHCDSTWMGDRVI |
| | TTSTRTWALPTYNNHLYRQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLSNGSQAVGRSSFYCPEYFPSQMLRTGNNFTFSYTFEDVPF |
| | HSSYAHSQSLDRLMNPLIDQYLYYLSTTNTPSGTTTQSRLQFSQAGASDIRDQSRNWLPG |
| | PCYRQQRVSKTSADNNNSEYSWTGATKYHLNGRDSLVNPGPAMASHKDNEEKFFPQSGVL |
| | IFGKQGSEKTNVDIEKVMITDEEEIRTTNPVATEQYGSVSTNLQRGNRQAATADVNTQGV |
| | LPGMVWQDRDVYLQGPIWAKIPHTDGHFHPSPLMGGFGPKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1459 | MAPGKKRPVEHSPVEPDSSSGTGKAGNQPARKRLNFGQTGDAD |
| | SVPDPQPLGQPPASPSGLGTNTMATGSGAPMADNNEGADGVGNSSGNWHCDSTWMGDRVV |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDLEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTAADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1460 | MAPGKKRPVEHSPVEPDSSSGTGKAGNQPARKRLNFGQTGDADS |
| | VPDPQPLGQPPAAPSGLGTNTMATGSGAPMADNNEGADGVGNSSGDWHCDSTWMGDRVIT |
| | TSTRTWALPTYNNHLYKQISSQSGASNDNHYFGYSTPWGYFDFNRFHCHFSPRDWQRLIN |
| | NNWGFRPKRLNLKLFNIQVKEVTQNDGTTTIANNLTSTVQVFTDLEYQLPYVLGSAHQGC |
| | LPPFPADVFMVPQYGYLTLNNGSQAVGRSSFYCLEYFPSQMLRTGNNFTFSYTFEDVPFH |
| | SSYAHSQSLDRLMNPLIDQYLYYLSRTNTPSGTTTQSRLQFSQAGASDIRDQSRNWLPGP |
| | CYRQQRVSKTAADNNNGEYSWTGATKYHLNGRDSLVNPGPAMASHKDDEEKFFPQSGVLI |
| | FGKQGSEKTNVDIEKVMITDEEEIRTTNPVATEQYGSVSTNLQSGNTRAATSDVNTQGVL |
| | PGMVWQDRDVYLQGPIWAKIPHTDGHFHPSPLMGGFGLKHPPPQILIKNTPVPANPSTTF |
| | SAAKFASFITQYSTGQVSVEIEWELQKENSKRWNPEIQYTSNYNKSVNVDFTVDTNGVYS |
| | EPRPIGTRYLTRNL |
| 1461 | MAPGKKRPVEHSPVEPDSSSGTGKAGNQPARKRLNFGQTGDAD |
| | SVPDPQPLGQPPAAPSGLGTNTMATGSGAPMADNNDGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVREVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWIGATKYHLNGRDSLVNPGPAMASHKDDEEKFFPQSGVL |
| | IFGKQGSEKTNVDIEKVMITDEEEIRTTNPVATEQYGSVSTNLQSGNTQASTADVNTQGV |
| | LPGMVWQDRDVYLQGPIWAKIPHTDGHFHPSPLMGGFGLKHPPPQILIKNTPVPANPSTT |
| | FSAAKFASFITQYSTGRVSVEIEWELQKENSKRWNPEIQYTSNYNKSVNVDFTVDINGVY |
| | SEPRPIGTRYLTRNL |
| 1462 | MAPGKKRPVEHSPAEPDSSSGTGKAGQQPARKRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1463 | MAPGKKRPVEHSLAEPDSSSGTGKAGQQPARRRLNFGQTGDAD |
| | SVPDPQPLGQPPAAPSGLGTNTMATGSGAPMADNNEGADGVGNSSGNWHCDSTWMGDRVI |
| | TTSTRTWALPTYNNHLYRQISSQSGASNDNHYFGYSTPWGYFDFNRFHCHFSPRDWQRLI |
| | NNNWGSRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLVDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1464 | MAPGKKRPVEPSPQRSPDSSAGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTETIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSCAHSQSSDRLMNPLIDQYLYYLSRTRSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| 1465 | MAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSGYAHSQSLDRLMNPLIDQYLYYLSRTQSTGGTQGTQQLLFSQAGPANMSAQAKNW |
| | LPGPCYRQQRVSTTLSQNNNSNFAWTGATKYHLNGRDSLVNPGVAMATHKDDEERFFPSS |
| | GVLMFGKQGAGRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |

TABLE 7
VP3 capsid proteins

| SEQ | |
|---|---|
| ID | |
| NO.: | VP3 Capsid Protein Sequence |
| 1466 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNVDADKVMITN |
| | EEEIKTTNPVATESYGQVATNHQGQSGRGDLGLSAQAAQTGWVQNQGILPGMVWQDRDVY |
| | LQGPIWAKIPHTDGNFHPSPLMGGFGMKHPPPQILIKNTPVPADPPTAFNKDKLNSFITQ |
| | YSTGQVSVEIEWELQKENSKRWNPEIQYTSNYYKSNNVEFAVNTEGVYSEPRPIGTRYLT |
| | RNLEFAVNTEGVYSEPRPIGTRYLTRNL |
| 1467 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WAAASSWALNGRNSLMNPGPAMASHKEGEDRFFPLSGSLIFGKQGTGRDNVDADKVMITN |
| | EEEIKTTNPVATESYGQVATNHQGQSGRGDLGLSAQAAQTGWVQNQGILPGMVWQDRDVY |
| | LQGPIWAKIPHTDGNFHPSPLMGGFGMKHPPPQILIKNTPVPADPPTAFNKDKLNSFITQ |
| | YSTGQVSVEIEWELQKENSKRWNPEIQYTSNYYKSNNVEFAVNTEGVYSEPRPIGTRYLT |
| | RNL |
| 1468 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNVDADKVMITN |
| | EEEIKTTNPVATESYGQVATNHQSGQSGRGDLGLSAQAATGWVQNQGILPGMVWQDRDVY |
| | LQGPIWAKIPHTDGNFHPSPLMGGFGMKHPPPQILIKNTPVPADPPTAFNKDKLNSFITQ |
| | YSTGQVSVEIEWELQKENSKRWNPEIQYTSNYYKSNNVEFAVNTEGVYSEPRPIGTRYLT |
| | RNL |
| 1469 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNVDADKVMITN |
| | EEEIKTTNPVATESYGQVATNHQGQSGRGDAVGVAQAAQTGWVQNQGILPGMVWQDRDVY |
| | LQGPIWAKIPHTDGNFHPSPLMGGFGMKHPPPQILIKNTPVPADPPTAFNKDKLNSFITQ |
| | YSTGQVSVEIEWELQKENSKRWNPEIQYTSNYYKSNNVEFAVNTEGVYSEPRPIGTRYLT |
| | RNL |
| 1470 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNVDADKVMITN |
| | EEEIKTTNPVATESYGQVATNHQSGQSGRGDAVGVAQAATGWVQNQGILPGMVWQDRDVY |
| | LQGPIWAKIPHTDGNFHPSPLMGGFGMKHPPPQILIKNTPVPADPPTAFNKDKLNSFITQ |
| | YSTGQVSVEIEWELQKENSKRWNPEIQYTSNYYKSNNVEFAVNTEGVYSEPRPIGTRYLT |
| | RNL |
| 1471 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WAAASSWALNGRNSLMNPGPAMASHKEGEDRFFPLSGSLIFGKQGTGRDNVDADKVMITN |
| | EEEIKTTNPVATESYGQVATNHQSGQSGRGDLGLSAQAATGWVQNQGILPGMVWQDRDVY |
| | LQGPIWAKIPHTDGNFHPSPLMGGFGMKHPPPQILIKNTPVPADPPTAFNKDKLNSFITQ |
| | YSTGQVSVEIEWELQKENSKRWNPEIQYTSNYYKSNNVEFAVNTEGVYSEPRPIGTRYLT |
| | RNL |
| 1472 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WAAASSWALNGRNSLMNPGPAMASHKEGEDRFFPLSGSLIFGKQGTGRDNVDADKVMITN |
| | EEEIKTTNPVATESYGQVATNHQGQSGRGDAVGVAQAAQTGWVQNQGILPGMVWQDRDVY |
| | LQGPIWAKIPHTDGNFHPSPLMGGFGMKHPPPQILIKNTPVPADPPTAFNKDKLNSFITQ |
| | YSTGQVSVEIEWELQKENSKRWNPEIQYTSNYYKSNNVEFAVNTEGVYSEPRPIGTRYLT |
| | RNL |
| 1473 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WAAASSWALNGRNSLMNPGPAMASHKEGEDRFFPLSGSLIFGKQGTGRDNVDADKVMITN |
| | EEEIKTTNPVATESYGQVATNHQSGQSGRGDAVGVAQAATGWVQNQGILPGMVWQDRDVY |
| | LQGPIWAKIPHTDGNFHPSPLMGGFGMKHPPPQILIKNTPVPADPPTAFNKDKLNSFITQ |
| | YSTGQVSVEIEWELQKENSKRWNPEIQYTSNYYKSNNVEFAVNTEGVYSEPRPIGTRYLT |
| | RNL |
| 1474 | MASGGGAPVADNNEGADGVGNASGNWHCDSQWLGDRVITTSTRTWALPTYNNHLYKQISS |
| | ASTGATNDNTYFGYSTPWGYFDFNRFHCHFSPRDWQRLINNNWGFRPKRLNFKLFNIQVK |
| | EVTDNNGVKTIANNLTSTVQVFTDSDYQLPYVLGSAHEGCLPPFPADVFMIPQYGYLTLN |
| | DGSQAVGRSSFYCLEYFPSQMLRTGNNFQFSYEFENVPFHSSYAHSQSLDRLMNPLIDQY |
| | LYYLSKTINGSGQNQQTLKFSVAGPSNMAVQGRNYIPGPSYRQQRVSTTVTQNNNSEFAW |
| | PGASSWALNGRNSLMNPGPAMASHKEGEDRFFPLSGSLIFGKQGTGRDNVDADKVMITNE |
| | EEIKTTNPVATESYGQVATNHQGQSGRGDLGLSAQAAQTGWVQNQGILPGMVWQDRDVYL |
| | QGPIWAKIPHTDGNFHPSPLMGGFGMKHPPPQILIKNTPVPADPPTAFNKDKLNSFITQY |
| | STGQVSVEIEWELQKENSKRWNPEIQYTSNYYKSNNVEFAVNTEGVYSEPRPIGTRYLTR |
| | NL |
| 1475 | MASGGGAPMADNNEGADGVGNASGNWHCDSTWLGDRVITTSTRTWALPTYNNHLYKQISN |
| | GTSGGATNDNTYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNVDADKVMITN |
| | EEEIKTTNPVATESYGQVATNHQGQSGRGDLGLSAQAAQTGWVQNQGILPGMVWQDRDVY |
| | LQGPIWAKIPHTDGNFHPSPLMGGFGMKHPPPQILIKNTPVPADPPTAFNKDKLNSFITQ |
| | YSTGQVSVEIEWELQKENSKRWNPEIQYTSNYYKSNNVEFAVNTEGVYSEPRPIGTRYLT |
| | RNL |
| 1476 | MASGGGAPMADNNEGADGVGNASGNWHCDSTWLGDRVITTSTRTWALPTYNNHLYKQISN |
| | GTSGGATNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNVDADKVMITN |
| | EEEIKTTNPVATESYGQVATNHQGQSGRGDLGLSAQAAQTGWVQNQGILPGMVWQDRDVY |
| | LQGPIWAKIPHTDGNFHPSPLMGGFGMKHPPPQILIKNTPVPADPPTAFNKDKLNSFITQ |
| | YSTGQVSVEIEWELQKENSKRWNPEIQYTSNYYKSNNVEFAVNTEGVYSEPRPIGTRYLT |
| | RNL |
| 1477 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNVDADKVMITN |
| | EEEIKTTNPVATESYGQVATNHQGQSGRGDLGLSAQAAQTGWVQNQGILPGMVWQDRDVY |
| | LQGPIWAKIPHTDGNFHPSPLMGGFGLKHPPPQILIKNTPVPADPPAEFSATKFASFITQ |
| | YSTGQVSVEIEWELQKENSKRWNPEVQYTSNYAKSANVDFTVDNNGLYTEPRPIGTRYLT |
| | RPL |
| 1478 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNVDADKVMITN |
| | EEEIKTTNPVATESYGQVATNHQSNSRGDYNSLAQAQTGWVQNQGILPGMVWQDRDVYLQ |
| | GPIWAKIPHTDGNFHPSPLMGGFGMKHPPPQILIKNTPVPADPPTAFNKDKLNSFITQYS |
| | TGQVSVEIEWELQKENSKRWNPEIQYTSNYYKSNNVEFAVNTEGVYSEPRPIGTRYLTRN |
| | L |
| 1479 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNVDADKVMITN |
| | EEEIKTTNPVATESYGQVATNHQSNSRGDYNSLAQAQTGWVQNQGILPGMVWQDRDVYLQ |
| | GPIWAKIPHTDGNFHPSPLMGGFGMKHPPPQILIKNTPVPADPPTAFNKDKLNSFITQYS |
| | TGQVSVEIEWELQKENSKRWNPEIQYTSNYYKSNNVEFAVNTEGVYSEPRPIGTRYLTRN |
| | L |
| 1480 | MASGGGAPVADNNEGADGVGSSSGNWHCDSQWLGDRVITTSTRTWALPTYNNHLYKQISN |
| | STSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTL |
| | NDGSQAVGRSSFYCLEYFPSQMLRTGNNFQFSYEFENVPFHSSYAHSQSLDRLMNPLIDQ |
| | YLYYLSKTINGSGQNQQTLKFSVAGPSNMAVQGRNYIPGPSYRQQRVSTTVTQNNNSEFA |
| | WPGASSWALNGRNSLMNPGPAMASHKEGEDRFFPLSGSLIFGKQGTGRDNVDADKVMITN |
| | EEEIKTTNPVATESYGQVATNHQSAQAQAQTGWVQNQGILPGMVWQDRDVYLQGPIWAKI |
| | PHTDGNFHPSPLMGGFGMKHPPPQILIKNTPVPADPPTAFNKDKLNSFITQYSTGQVSVE |
| | IEWELQKENSKRWNPEIQYTSNYYKSNNVEFAVNTEGVYSEPRPIGTRYLTRNL |
| 1481 | MASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTQSRLQFSQAGASDIRDQSRNWLPG |
| | PCYRQQRVSKTSADNNNSEYSWTGATKYHLNGRDSLVNPGPAMASHKDDEEKFFPQSGVL |
| | IFGKQGSEKTNVDIEKVMITDEEEIRTTNPVATEQYGSVSTNLQGGNTQAATADVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1482 | MASGSGAPMADNNEGADGVGNSSGNWYCDSTWMGDRVITTSTRTWALPTYNNHLYKQISS |
| | QSGASNDNHYFGYSTPWGYFDFNRFHCHFSPRDWQRLINNNWGFRPKRLSFKLFNIQVKE |
| | VTQNDGTTTIANNLTSTVQVFTDSEYQLPYVLGSAHQGCLPPFPADVFMVPQYGYLTLNN |
| | GSQAVGRSSFYCLEYFPSQMLRTGNNFTFSYTFEDVPFHSSYAHSQSLDRLMNPLIDQYL |
| | YYLSRTNTPSGTTTMSRLQFSQAGASDIRDQSRNWLPGPCYRQQRVSKTAADNNNSDYSW |
| | TGATKYHLNGRDSLVNPGPAMASHKDDEEKYFPQSGVLIFGKQDSGKTNVDIEKVMITDE |
| | EEIRTTNPVATEQYGSVSTNLQSGNTQAATSDVNTQGVLPGMVWQDRDVYLQGPIWAKIP |
| | HTDGHFHPSPLVGGFGLKHPPPQILIKNTPVPANPSTTFSAAKFASFITQYSTGQVSVEI |
| | EWELQKENSKRWNPEIQYTSNYNKSVNVDFTVDTNGVYSEPRPIGTRYLTRNL |
| 1483 | MASGSGAPMADNNEGADGVGNSSGNWHCDSTWMGDRVITTSTRTWALPTYNNHLYKQISS |
| | QSGASNDNHYFGYSTPWGHFDFNRFHCHFSPRDWQRLINNNWGFRPKRLSFKLFNIQVKE |
| | VTQNDGTTTIANNLTSTVQVFTDSEYQLPYVLGSAHQGCLPPFPADVFMVPQYGYLTLNN |
| | GSQAVGRSSFYCLEYFPSQMLRTGNNFTFSYTFEDVPFHSSYAHSQSLDRLMNPLIDQYL |
| | YYLSRTNTPSGTTTMSRLQFSQAGASDIRDQSRNWLPGPCYRQQRVSKTAADNNNSDYSW |
| | TGATKYHLNGRDSLVNPGPAMASHKDDEEKYFPQSGVLIFGKQDSGKTNVDIEKVMITDE |
| | EEIRTTNPVATEQYGSVSTNLQSGNTQAATSDVNTQGVLPGMVWQDRDVYLQGPIWAKIP |
| | HTDGHFHPSPPMGGFGLKHPPPQILIKNTPVPANPSTTFSAAKFASFITQYSTGQVSVEI |
| | EWELQKENSKRWNPEIQYTSNYNKSVNVDFTVDTNGVYSEPRPIGARYLTRNL |
| 1484 | MASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1485 | MASGSGAPMADNNEGADGVGNSSGNWHCDSTWMGDRVITTSTRTWALPTYNNHLYKQISS |
| | QSGASNDNHYFGYSTPWGYFDFNRFHCHFSPRDWQRLINNNWGFRPKRLSFKLFNIQVKE |
| | VTQNDGTTTIANNLTSTVQVFTDSEYQLPYVLGSAHQGCLPPFPADVFMVPQYGYLTLNN |
| | GSQAVGRSSFYCLEYFPSQMLRTGNNFTFSYTFEDVPFHSSYAHSQSLDRLMNPLIDQYL |
| | YYLSRTNTPSGTTTMSRLQFSQAGASDIRDQSRNWLPGPCYRQQRVSKTAADNNNSDYSW |
| | TGATKYHLNGRDSLVNPGPAMASHKDDEEKYFPQSGVLIFGKQDSGKTNVDIEKVMITDE |
| | EEIRTTNPVATEQYGSVSTNLQSGNTQAATSDVNTQGVLPGMVWQDRDVYLQGPIWAKIP |
| | HTDGHFHPSPLMGGFGLKHPPPQILIKNTPVPANPSTTFSAAKFASFITQYSTGQVSVEI |
| | EWELQKENSKRWNPEIQYTSNYNKSVNVDFTVDTNGVYSEPRPIGTRYLTRNL |
| 1486 | MASGSGAPMADNNEGADGVGNSSGNWHCDSTWMGDRVI |
| | TTSTRTWALPTCNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTYLQSGNTQAATSDVNTQGV |
| | LPGMVWQDRDVYLRGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDINGVY |
| | SEPRPIGTRYLTRNL |
| 1487 | MASGSGAPMADNNEGADGVGNSSGNWHCDSTWMGDRVI |
| | TTSTRTWALPTCNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTYLQSGNTQAATSDVNTQGV |
| | LPGMVWQDRDVYLRGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1488 | MASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFTFSYTFEDVPF |
| | HSSYAHSQSLDRLMNPLIDQYLYYLSRTNTPSGTTTMSRLQFSQAGASDIRDQSRNWLPG |
| | PCYRQQRVSKTAADNNNSDYSWTGATKYHLNGRDSLVNPGPAMASHKDDEEKYFPQSGVL |
| | IFGKQDSGKTNVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVDTNGVY |
| | SEPRPIGTRYLTRNL |
| 1489 | MASGSGAPMADNNEGADGVGNSSGNWHCDSTWMGDRVI |
| | TTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSGYQLPYVLGSAHQG |
| | CLPPFPADVFMVPQYGYLTLNNGSQAVGRSSFYCLEYFPSQMLRTGNNFTFSYTFEDVPF |
| | HSSYAHGQSLDRLMNPLIDQYLYYLSRTNTPSGTTTMSRLQFSQAGASDVRDQSRNWLPG |
| | PCYRQQRVSKTAADNNNSDYSWTGATKYHLNGRDSLVNPGPAMASHKDDEEKYFPQSGVL |
| | VFGKQDSGKTNVDIEKVMITDEEEIRTTNPAATEQYGSVSTNLQSGNTQAATSDVNTQGV |
| | LPGMVWQDRDVYLQGPIWAKIPHTDGHFHPSPLMGGFGLKHPPPQILIKNTPVPANPSTT |
| | FSAAKFVSFITQYSTGQVSVEIEWELQKENSKRWNPEIQYTSNYNKSVNVDFTVDTNGVY |
| | SEPRPIGTRYLTRNL |
| 1490 | MASGSGAPVADNNEGADGVGNSSGNWHCDSQWLGDRVI |
| | TTSTRTWALPTYNNHLYKQISSQSGASNDNHYFGYSTPWGYFDFNRFHCHFSPRDWQRLI |
| | NNSWGFRPKRLNFKLFNIQVKEVTQNDGTTTIANNLTSTVQVFTDSEYQLPYVLGSAHQG |
| | CLPPFPADVFMVPQYGYLTLNNGSQAVGRSSFYCLEYFPSQMLRTGNNFQFSYTFEDVPF |
| | HSSYAHSQSLDRLLNPLIDQYLYYLNKTQSNSGTLQQSRLLFSQAGPTSMSLQAKNWLPG |
| | PCYRQQRLSKQANDNNNSNFPWTAATKYHLNGRDSLVNPGPAMASHKDDEEKFFPMHGTL |
| | IFGKQGTNANDADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKNTPVPANPPTN |
| | FSSSKFASFITQYSTGQVSVEIEWELQKENSKRWNPEIQYTSNYNKSVNVDFTVDINGVY |
| | SEPRPIGTRYPTRNL |
| 1491 | MASGGGAPMADNNEGADGVGNASGNWHCDSTWLGDRVI |
| | TTSTRTWALPTYNNHLYKQISSTSTGASNDNHYFGYGTPWGYFDFNRFHCHFSPRDWQRL |
| | INNNWGFRPKRLNFKLFNIQVEEVTTNDGVTTIANNLTSTVQVFSDSEYQLPYVLGSAHQ |
| | GCLPPFPADVFMIPQYGYLTLNNGSQAVGRSSFYCLEYFPSQMLRTGNNFTFSYTFEEVP |
| | FHSSYAHSQSLDRLMNPLIDQYLYYLNRTQNQSGSAQNKDLLFSRGSPAGMSVQPKNWLP |
| | GPCYRQQRVSKTKTDNNNSNFTWTGASKYNLNGRESIINPGTAVASHKDDEDKFFPMSGV |
| | MIFGKESAGASSTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIKNTPVPANPPA |
| | EFSATKFASFITQYSTGQVSVEIEWELQKENSKRWNPEVQYTSNYAKSANVDFTVDNNGL |
| | YTEPRPIGTRYLTRPL |
| 1492 | MASGGGAPMADNNEGADGVGNASGNWHCDSTWLGDRVI |
| | TTSTRTWALPTYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQLPYVLGSAHQ |
| | GCLPPFPADVFMIPQYGYLTLNNGSQAVGRSSFYCLEYFPSQMLRTGNNFTFSYTFEEVP |
| | FHSSYAHSQSLDRLMNPLIDQYLYYPNRTQNQSGSAQNKDLLFSRGSPAGMSVQPKNWLP |
| | GPCYRQQRVSKTKTDNNNSNFTWTGASKYNLNGRESIINPGTAMASHKDDEDKFFPMSGV |
| | MIFGKESAGASNTALDNVMITDEEEIKATNPVATERFGTVAVNFQSSSTDPATGDVHAMG |
| | ALPGMVWQGRDVYLQGPIWAKIPHTDGHFHPSPLMGGFGLKNPPPQILIKNTPVPANPPA |
| | EFSATKFASFITQYSTGQVSVEIEWELQKENSKRWNPEVQYTSNYAKSANVDFTVDNNGL |
| | YTEPRPIGTRYLTRPL |
| 1493 | MASGGGAPMADNNEGADGVGNASGNWHCDSTWLGDRVI |
| | TTSTRTWALPTYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQLPYVLGSAHQ |
| | GRLPPFPADVFMIPQYGYLTLNNGSQAVGRSSSYCLEYFPSQMLRTGNNFTFSYTFEEVP |
| | LHSSCAHSQSLDRLMNPLIDQYLYYLNRTQNQSGSAQNRDLLFSRGSPAGMSVQPKNWLP |
| | GPCYRQQRVSKTKTDNNNSNFTWTGASKYNLNGRESIINPGTAMASHKDDEDKFFPMSGV |
| | MIFGKESAGASNTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIKNTPVPANPPA |
| | EFSATKFASFITQYSAGQVSVEIEWELQKENSKRWNPEVQYTSNYAKSANVDFTVDNNGL |
| | YTEPRPIGTRYLTRPL |
| 1494 | MASGGGAPMADNNEGADGVGNASGNWHCDSTWLGDRVI |
| | TTSTRTWALPTYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQLPYVLGSAHQ |
| | GCLPPFPADVFMIPQYGYLTLNNGSQAVGRSSFYCLEYFPSQMLRTGNNFTFSYTFEEVP |
| | FHSSYAHSQSLDRLMNPLIDQYLYYLNRTQNQSGSAQNKDLLFSRGSPAGMSVQPKNWLP |
| | GPCYRQQRVSKTKTDNNNSNFTWTGASKYNLNGRESIINPGTAMASHKDDEDKFFPMSGV |
| | MIFGKESAGASNTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIKNTPVPANPPA |
| | EFSATKFASFITQYSTGQVSVEIEWELQKENSKRWNPEVQYTSNYAKSANVDFTVDNNGL |
| | YTEPRPIGTRYLTRPL |

### BRIEF DESCRIPTION OF THE DRAWINGS

FIG. 1A-C show results from KT-430 administration on survival and growth in Mtm1 KO Mice.

FIG. 2 shows results from KT-430 administration on muscle function in Mtm1 KO

### Mice.

FIG. 3 shows the biodistribution of KT-430 in Mtm1 KO Mice

FIG. 4 shows dose-dependent expression of hMTM1 mRNA following treatment of KT-430 in Mtm1 KO Mice.

FIG. 5 shows the dose-dependent expression of MTM1 Protein Following Treatment of KT-430 in Mtm1 KO Mice.

### DETAILED DESCRIPTION

The present invention provides a novel muscle-tropic viral vector that achieves MTM1 expression in skeletal muscle with reduced exposure of the vector in liver tissue. Advantageously, by increasing expression of MTM1 in skeletal muscle, the vector of the present allows for administration of a dose of the composition comprising the vector that substantially reduces the exposure of non-target tissues to the composition.

### XLMTM and AT132

XLMTM results from pathogenic variants in the myotubularin (MTM1) gene, which encodes the protein myotubularin, a ubiquitously expressed lipid phosphatase that regulates intracellular membrane trafficking and vesicular transport, and whose function is required for the normal development, maturation, and maintenance of skeletal muscleReductions in functional myotubularin are associated with the profound skeletal muscle weakness responsible for the most prominent clinical manifestations of this disease.

MTM1 is a member of a large evolutionarily conserved family of myotubularin phosphatases. It is a ubiquitously expressed lipid phosphatase that dephosphorylates the D3 phosphate of the inositol ring of two types of phosphoinositides: the phosphatidylinositol 3-phosphate (PtdIns3P) and the phosphatidylinositol 3,5-bisphophate (PtdIns(3,5) P2). MTM1 regulates numerous cellular processes, including vesicle sorting through endosomal compartments, excitation-contraction coupling and T-tubule organization in muscle. Loss of function mutations in the MTM1 gene cause defects in these processes, which are believed to underly the severe motor dysfunction and histological defects in XLMTM diseased muscle.

Loss of MTM1 gene function in the mouse and dog is associated with similar functional and histological defects as in human children with XLMTM, including reduced survival, impaired motor function, muscle atrophy, reductions in contractile strength and associated histological defects in organelle organization including central nuclei, mislocalized mitochondria and T-tubule disorganization. The human MTM1 coding sequence is described in SEQ ID NO: 3. Restoration of functional myotubularin via AAV8-mediated gene therapy to express full-length murine MTM1 under the control of the desmin promoter has been shown to improve survival, body weight gain, muscle contractile force, motor function and histological defects in organelle mislocalization when administered by intramuscular or intravenous (IV) injection into Mtm1 knockout (KO) mice.

Without being bound to a mechanism of action, it is thought that XLMTM produces an abnormal hepatic substrate which is itself non-severe, but susceptible to additional insult. Hepatic abnormalities including cholestasis are part of the natural history of XLMTM. Hepatic manifestations are less prominent than the skeletal and respiratory muscle weakness which is the predominant and most frequently fatal clinical manifestation; however, hepatic manifestations may be more clinically relevant and increasingly recognized after the emergence of hepatotoxicity in the AT132 program. Cholestatic liver failure has not been reported to be part of the natural history of the disease, but there are case reports of substantial elevations in bilirubin in the setting of stressors such as respiratory infections, consistent with the existence of a predisposition to cholestasis which can be mild or even subclinical in the absence of clinical stress. It is presently unknown whether cholestasis or a predisposition to cholestasis may associated with other variables such as the specific MTM1 mutation, neonatal jaundice, and other hepatic abnormalities such as peliosis.

Relevant to the presently proposed program, the clinical doses of AT132 used to date have been high (1 E14 and 3 E14 vg/kg), similar to those used in other vector-based gene therapy programs, presumably in order to compensate for suboptimal biodistribution and/or expression in tissue(s) of interest due to use of unselected naturally occurring capsids. Interim data from ASPIRO reported as of Jan. 29, 2021 indicated that severe treatment-emergent hepatobiliary events were more common in the high-dose group (5 of 17 participants in the high-dose group vs. 0 out of 6 in the low-dose group), though presumably at least one subsequent event occurred in the low-dose group in the study participant reported in September 2021. Recently published biopsy and autopsy data indicated that study participants who experienced fatal hepatic events after treatment with AT132 had very high vector copy numbers (VCN) in the liver, in both absolute terms and relative to heart and skeletal muscle. In contrast, little to no MTM1 or myotubularin was identified in liver tissue. Without being bound to a mechanism of action, these findings suggest that the liver toxicity may be driven by the capsid rather than the transgene, that dose is a factor, and that reduced hepatic exposure to capsid may reduce the risk of vector-based gene therapy for boys with XLMTM. For example, other types of adverse events associated with vector-based gene therapies (such as complement activation and its sequelae) have not been reported in the ASPIRO trial.

### Adeno Associated Virus Vector

AAVs are particularly appropriate viral vectors for delivery of genetic material into mammalian cells. AAVs are not known to cause disease in mammals and cause a very mild immune response. Additionally, AAVs are able to infect cells in multiple stages whether at rest or in a phase of the cell replication cycle. Advantageously, AAV DNA is not regularly inserted into the host's genome at random sites, reducing the oncogenic properties of this vector.

AAVs have been engineered to deliver a variety of treatments, especially for genetic disorders caused by single nucleotide polymorphisms (“SNP”). Genetic diseases that have been studied in conjunction with AAV vectors include Cystic fibrosis, hemophilia, arthritis, macular degeneration, muscular dystrophy, Parkinson's disease, congestive heart failure, and Alzheimer's disease. The AAV can be used as a vector to deliver engineered nucleic acid to a host and utilize the host's own ribosomes to transcribe that nucleic acid into the desired proteins. Sec, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

AAVs are small, replication-defective, nonenveloped viruses that infect humans and other primate species and have a linear single-stranded DNA genome. Naturally occurring AAV serotypes exhibit liver tropism. As a result, transfection of non-liver tissue with traditional AAV vectors is impeded by the virus's natural liver tropism. Moreover, because the liver acts to break down substances delivered to a subject, transfection of non-liver tissue with unmodified AAV vectors requires higher dosing to provide sufficient viral load to overcome the liver and reach non-liver tissue. More than 30 naturally occurring serotypes of AAV are available. Many natural variants in the AAV capsid exist. AAV serotypes include, but are not limited to, AAV serotypes AAV1, AAV2, AAV3, AAV3B, AAV4, AAV5, AAV6, AAV7, AAV8, AAV9, AAV10, AAV11, AAV 12, AAV13. AAVs may be engineered using conventional molecular biology techniques, making it possible to optimize these particles, for example, for cell specific delivery, for minimizing immunogenicity, for tuning stability and particle lifetime, for efficient degradation, for accurate delivery to the nucleus. AAV vectors can be specifically targeted to one or more types of cells by choosing the appropriate combination of AAV serotype, promoter, and delivery method.

Previous approaches to identify AAV sequences correlated with tropism have relied upon the comparison of highly related extant serotypes with distinct characteristics, random domain swaps between unrelated serotypes, or consideration of higher-order structure, to identify motifs that define liver tropism. For example, mapping determinants of AAV tropism have been carried out by comparing highly related serotypes. One such example is the single-amino acid change (E531K) between AAV1 and AAV6 that improves murine liver transduction in AAV1. See Wu et al. (2006) J. Virol., 80 (22): 11393-7, incorporated by reference herein. Another example is a reciprocal domain swap between AAV2 and AAV8 that alters tropism, but fails to define any robust specific tissue-targeting motifs. See Raupp et al. (201) J. Virol., 86 (17): 9396-408, incorporated by reference herein. Further, global consideration of structure has only highlighted gross differences between better- or worse-liver-transducers that are more observational than useful in practice. Nam et al (2007) J. Virol., 81 (22): 12260-71.

AAVs exhibiting modified tissue tropism that may be used with the present invention are described in U.S. Pat. Nos. 9,695,220, 9,719,070; 10,119,125; 10,526,584; U.S. Patent Application Publication No. 2018-0369414; U.S. Patent Application Publication No. 2020-0123504; U.S. Patent Application Publication No. 2020-0318082; PCT International Patent Application Publication No. WO 2015/054653; PCT International Patent Application Publication No. WO 2016/179496; PCT International Patent Application Publication No. WO 2017/100791; and PCT International Patent Application Publication No. WO 2019/217911, the entirety of the contents of each of which are incorporated by reference herein.

The AAV vector or system thereof may include one or more regulatory molecules, such as promoters, enhancers, repressors and the like. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the muscle specific promoter can drive expression of an engineered AAV capsid polynucleotide.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue-, and/or organ-specific tropism.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5, AAV-9 or any combination thereof. One can select the AAV of the AAV with regard to the cells to be targeted; e.g., one can select AAV serotypes 1, 2, 5, 9 or a hybrid capsid AAV-1, AAV-2, AAV-5, AAV-9 or any combination thereof for targeting brain and/or neuronal cells; and one can select AAV-4 for targeting cardiac tissue; and one can select AAV-8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

It will be appreciated that while the different serotypes can provide some level of cell, tissue, and/or organ specificity, each serotype still is multi-tropic and thus can result in tissue-toxicity if using that serotype to target a tissue that the serotype is less efficient in transducing. Thus, in addition to achieving some tissue targeting capacity via selecting an AAV of a particular serotype, it will be appreciated that the tropism of the AAV serotype can be modified by an engineered AAV capsid described herein. As described elsewhere herein, variants of wild-type AAV of any serotype can be generated via a method described herein and determined to have a particular cell-specific tropism, which can be the same or different as that of the reference wild-type AAV serotype. In some embodiments, the cell, tissue, and/or specificity of the wild-type serotype can be enhanced (e.g., made more selective or specific for a particular cell type that the serotype is already biased towards). For example, wild-type AAV-9 is biased towards muscle and brain in humans (see e.g., Srivastava. 2017. Curr. Opin. Virol. 21:75-80.) By including an engineered AAV capsid and/or capsid protein variant of wild-type AAV-9 as described herein, the tropism for nervous cells might be reduced or eliminated and/or the muscle specificity increased such that the nervous specificity appears reduced in comparison, thus enhancing the specificity for muscle as compared to the wild-type AAV-9. As previously mentioned, inclusion of an engineered capsid and/or capsid protein variant of a wild-type AAV serotype can have a different tropism than the wild-type reference AAV serotype. For example, an engineered AAV capsid and/or capsid protein variant of AAV-9 can have specificity for a tissue other than muscle or brain in humans.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same specificity issues as with the non-hybrid wild-type serotypes previously discussed.

Advantages achieved by the wild-type based hybrid AAV systems can be combined with the increased and customizable cell-specificity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV-9 serotype that is used to package a genome that contains components (e.g., rep elements) from an AAV-2 serotype. As with wild-type based hybrid AAVs previously discussed, the tropism of the resulting AAV particle will be that of the engineered AAV capsid.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered AAV capsid polynucleotide(s)).

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of a engineered AAV capsid system described herein are as used in the foregoing documents, such as International Patent Application Publications WO WO 2021/050974 and WO 2021/077000 and PCT International Application No. PCT/US2021/042812, the contents of which are incorporated by reference herein.

Additional AAV vectors are described in International Patent Application Publication WO 2019/2071632, the contents of which are incorporated by reference herein.

Further AAV vectors are described in International Patent Application Publications WO 2020/086881 and WO 2020/235543, the contents of each of which are incorporated by reference herein.

Further AAV vectors are described in International Patent Application Publications WO 2005/033321; WO 2006/110689; WO 2007/127264; WO 2008/027084; WO 2009/073103; WO 2009/073104; WO 2009/105084; WO 2009/134681; WO 2009/136977; WO 2010/051367; WO 2010/138675; WO 2001/038187; WO 2012/112832; WO 2015/054653; WO 2016/179496; WO 2017/100791; WO 2017/019994; WO 2018/209154; WO 2019/067982; WO 2019/195701; WO 2019/217911; WO 2020/041498; WO 2020/210839; U.S. Pat. Nos. 7,906,111; 9,737,618; 10,265,417; 10,485,883; 10,695,441; 10,722,598; 8,999,678; 10,301,648; 10,626,415; 9,198,984; 10,155,931; 8,524,219; 9,206,238; 8,685,387; 9,359,618; 8,231,880; 8,470,310; 9,597,363; 8,940,290; 9,593,346; 10,501,757; 10,786,568; 10,973,928; 10,519,198; 8,846,031; 9,617,561; 9,884,071; 10,406,173; 9,596,220; 9,719,010; 10,117,125; 10,526,584; 10,881,548; 10,738,087; U.S. Patent Publication No. 2011-023353; U.S. Patent Publication No. 2019-0015527; U.S. Patent Publication No. 2020-155704; U.S. Patent Publication No 2017-0191079; U.S. Patent Publication No. 2019-0218574; U.S. Patent Publication No. 2020-0208176; U.S. Patent Publication No. 2020-0325491; U.S. Patent Publication No. 2019-0055523; U.S. Patent Publication No. 2020-0385689; U.S. Patent Publication No. 2009-0317417; U.S. Patent Publication No. 2016-0051603; U.S. Patent Publication No. 2016-00244783; U.S. Patent Publication No. 2017-0183636; U.S. Patent Publication No. 2020-0263201; U.S. Patent Publication No. 2020-0101099; U.S. Patent Publication No. 2020-0318082; U.S. Patent Publication No. 2018-0369414; U.S. Patent Publication No. 2019-0330278; U.S. Patent Publication No. 2020-0231986, the contents of each of which are incorporated by reference herein.

### Capsid Protein

The capsid protein is the shell or coating of the virus that enables its delivery into the host. Without the protein, the nucleic acids would be destroyed by the host without entering into the host cells and beginning transcription and translation. The capsid protein may be in the natural conformation of a naturally occurring AAV, or it may be modified.

In certain example embodiments, the AAV capsid protein is an engineered AAV capsid protein having reduced or eliminated uptake in a non-muscle cell as compared to a corresponding wild-type AAV capsid polypeptide.

In some embodiments, the engineered AAV capsid encoding polynucleotide can be included in a polynucleotide that is configured to be an AAV genome donor in an AAV vector system that can be used to generate engineered AAV particles described elsewhere herein. In some embodiments, the engineered AAV capsid encoding polynucleotide can be operably coupled to a poly adenylation tail. In some embodiments, the poly adenylation tail can be an SV40 poly adenylation tail. In some embodiments, the AAV capsid encoding polynucleotide can be operably coupled to a promoter. In some embodiments, the promoter can be a tissue specific promoter. In some embodiments, the tissue specific promoter is specific for muscle (e.g., cardiac, skeletal, and/or smooth muscle), neurons and supporting cells (e.g., astrocytes, glial cells, Schwann cells, etc.), fat, spleen, liver, kidney, immune cells, spinal fluid cells, synovial fluid cells, skin cells, cartilage, tendons, connective tissue, bone, pancreas, adrenal gland, blood cell, bone marrow cells, placenta, endothelial cells, and combinations thereof. In some embodiments, the promoter can be a constitutive promoter. Suitable tissue specific promoters and constitutive promoters are discussed elsewhere herein and are generally known in the art and can be commercially available. Suitable muscle specific promoters include, but are not limited to CK8, MHCK7, Myoglobin promoter (Mb), Desmin promoter, muscle creatine kinase promoter (MCK) and variants thereof, and SPc5-12 synthetic promoter.

Described herein are various embodiments of engineered viral capsids, such as adeno-associated virus (AAV) capsids, that can be engineered to confer cell-specific tropism, such as muscle specific tropism, to an engineered viral particle. Engineered viral capsids can be lentiviral, retroviral, adenoviral, or AAV capsids. The engineered capsids can be included in an engineered virus particle (e.g., an engineered lentiviral, retroviral, adenoviral, or AAV virus particle), and can confer cell-specific tropism, reduced immunogenicity, or both to the engineered viral particle. The engineered viral capsids described herein can include one or more engineered viral capsid proteins described herein. The engineered viral capsids described herein can include one or more engineered viral capsid proteins described herein that can contain a muscle-specific targeting moiety containing or composed of an n-mer motif described elsewhere herein.

The engineered viral capsid and/or capsid proteins can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

The engineered viral capsids can be variants of wild-type viral capsid. For example, in some embodiments, the engineered AAV capsids can be variants of wild-type AAV capsids. In some embodiments, the wild-type AAV capsids can be composed of VP1, VP2, VP3 capsid proteins or a combination thereof. In other words, the engineered AAV capsids can include one or more variants of a wild-type VP1, wild-type VP2, and/or wild-type VP3 capsid proteins. In some embodiments, the serotype of the reference wild-type AAV capsid can be AAV-1, AAV-2, AAV-3, AAV-4, AAV-5, AAV-6, AAV-8, AAV-9 or any combination thereof. In some embodiments, the serotype of the wild-type AAV capsid can be AAV-9. The engineered AAV capsids can have a different tropism than that of the reference wild-type AAV capsid.

The engineered viral capsid can contain 1-60 engineered capsid proteins. In some embodiments, the engineered viral capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered viral capsid can contain 0-59 wild-type viral capsid proteins. In some embodiments, the engineered viral capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type viral capsid proteins.

In some embodiments, the engineered AAV capsid can contain 1-60 engineered capsid proteins. In some embodiments, the engineered AAV capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered AAV capsid can contain 0-59 wild-type AAV capsid proteins. In some embodiments, the engineered AAV capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type AAV capsid proteins.

In some embodiments, the engineered viral capsid protein can have an n-mer amino acid motif, where n can be at least 3 amino acids. In some embodiments, n can be 3, 4, 5, 6, 7, 8, 9, 10, 11, 12, 13, 14, or 15 amino acids. In some embodiments, an engineered AAV capsid can have a 6-mer or 7-mer amino acid motif. In some embodiments, the n-mer amino acid motif can be inserted between two amino acids in the wild-type viral protein (VP) (or capsid protein). In some embodiments, the n-mer motif can be inserted between two amino acids in a variable amino acid region in a viral capsid protein.

In some embodiments, the n-mer motif can be inserted between two amino acids in a variable amino acid region in an AAV capsid protein. The core of each wild-type AAV viral protein contains an eight-stranded beta-barrel motif (betaB to betaI) and an alpha-helix (alphaA) that are conserved in autonomous parvovirus capsids (see e.g., DiMattia et al. 2012. J. Virol. 86 (12): 6947-6958). Structural variable regions (VRs) occur in the surface loops that connect the beta-strands, which cluster to produce local variations in the capsid surface. AAVs have 12 variable regions (also referred to as hypervariable regions) (see e.g., Weitzman and Linden. 2011. “Adeno-Associated Virus Biology.” In Snyder, R. O., Moullier, P. (eds.) Totowa, NJ: Humana Press). In some embodiments, one or more n-mer motifs can be inserted between two amino acids in one or more of the 12 variable regions in the wild-type AVV capsid proteins. In some embodiments, the one or more n-mer motifs can be each be inserted between two amino acids in VR-I, VR-II, VR-III, VR-IV, VR-V, VR-VI, VR-VII, VR-III, VR-IX, VR-X, VR-XI, VR-XII, or a combination thereof. In some embodiments, the n-mer can be inserted between two amino acids in the VR-III of a capsid protein. In some embodiments, the engineered capsid can have an n-mer inserted between any two contiguous amino acids between amino acids 262 and 269, between any two contiguous amino acids between amino acids 327 and 332, between any two contiguous amino acids between amino acids 382 and 386, between any two contiguous amino acids between amino acids 452 and 460, between any two contiguous amino acids between amino acids 488 and 505, between any two contiguous amino acids between amino acids 545 and 558, between any two contiguous amino acids between amino acids 581 and 593, between any two contiguous amino acids between amino acids 704 and 714 of an AAV9 viral protein. In some embodiments, the engineered capsid can have an n-mer inserted between amino acids 588 and 589 of an AAV9 viral protein. In some embodiments, the engineered capsid can have a 7-mer motif inserted between amino acids 588 and 589 of an AAV9 viral protein. In other embodiments, the motif inserted is a 10-mer motif, with replacement of amino acids 586-88 and an insertion before 589. SEQ ID NO. 1 is a reference AAV9 capsid sequence for at least referencing the insertion sites discussed above. It will be appreciated that n-mers can be inserted in analogous positions in AAV viral proteins of other serotypes. In some embodiments as previously discussed, the n-mer(s) can be inserted between any two contiguous amino acids within the AAV viral protein and in some embodiments the insertion is made in a variable region.

In some embodiments, the first 1, 2, 3, or 4 amino acids of an n-mer motif can replace 1, 2, 3, or 4 amino acids of a polypeptide into which it is inserted and preceding the insertion site. In some embodiments, the amino acids of the n-mer motif that replace 1 or more amino acids of the polypeptide into which the n-mer motif is inserted come before or immediately before an “RGD” in an n-mer motif. For example, in one or more of the 10-mer inserts shown in e.g., Tables 2-3, the first three amino acids shown can replace 1-3 amino acids into a polypeptide to which they may be inserted. Using an AAV as another non-limiting example, one or more of the n-mer motifs can be inserted into e.g., and AAV9 capsid prolylpeptide between amino acids 588 and 589 and the insert can replace amino acids 586, 587, and 588 such that the amino acid immediately preceding the n-mer motif after insertion is residue 585. It will be appreciated that this principle can apply in any other insertion context and is not necessarily limited to insertion between residues 588 and 589 of an AAV9 capsid or equivalent position in another AAV capsid. It will further be appreciated that in some embodiments, no amino acids in the polypeptide into which the n-mer motif is inserted are replaced by the n-mer motif.

In some embodiments, the AAV capsids or other viral capsids or compositions can be muscle-specific. In some embodiments, muscle-specificity of the engineered AAV or other viral capsid or other composition is conferred by a muscle specific n-mer motif incorporated in the engineered AAV or other viral capsid or other composition described herein. While not intending to be bound by theory, it is believed that the n-mer motif confers a 3D structure to or within a domain or region of the engineered AAV capsid or other viral capsid or other composition such that the interaction of the viral particle or other composition containing the engineered AAV capsid or other viral capsid or other composition described herein has increased or improved interactions (e.g., increased affinity) with a cell surface receptor and/or other molecule on the surface of a muscle cell. In some embodiments, the cell surface receptor is AAV receptor (AAVR). In some embodiments, the cell surface receptor is a muscle cell specific AAV receptor. In some embodiments, the cell surface receptor or other molecule is a cell surface receptor or other molecule selectively expressed on the surface of a muscle cell. In some embodiments, the cell surface receptor or molecule is an integrin or dimer thereof. In some embodiments, the cell surface receptor or molecule is an Vb6 integrin heterodimer.

In some embodiments, a muscle specific engineered viral particle or other composition described herein containing the muscle-specific capsid, n-mer motif, or muscle-specific targeting moiety described herein can have an increased uptake, delivery rate, transduction rate, efficiency, amount, or a combination thereof in a muscle cell as compared to other cells types and/or other virus particles (including but not limited to AAVs) and other compositions that do not contain the muscle-specific n-mer motif of the present invention.

First- and second-generation muscle specific AAV capsids were developed using a muscle specific promoter and the resulting capsid libraries were screened in mice and non-human primates as described elsewhere herein and/or in e.g., U.S. Provisional Application Ser. Nos. 62/899,453, 62/916,207, 63/018,454, and 63/242,008. First and second generation myoAAV capsids were further optimized in mice and non-human primates as previously described to generate enhanced myoAAV capsids.

Tables 1 and 2 show the top hits of enhanced muscle specific n-mer motifs and their encoding sequence in rank order within each table. Enhanced MyoAAV (cMyoAAV) capsid variants can transduce mouse muscle more effectively as compared to the first generation MyoAAV after systemic delivery. First and second generation myoAAV capsid variants are dependent on the aVb6 integrin heterodimer for transduction of human primary myotubes.

Tables 3 and 4 show top-ranking capsid variants produced in rounds of directed evolution of capsid variants for skeletal muscle specificity. As shown in the Tables above with respect to those variant n-mer inserts containing P-motifs, the first three amino acids of the variant sequences shown are amino acids that replaced amino acids corresponding to positions 596, 597, and 598 of an AAV9 capsid polypeptide. Thus, the P-motif, for example, was inserted between amino acids at positions 598 and 599 of an AAV9 vector.

An AAV may further comprise may comprise vp1, vp2, and vp3 capsid proteins. The amino acid sequence of the vp1 capsid protein of vectors of the present invention may be selected from the sequences in Table 5, the amino acid sequence vp2 capsid protein may be selected from the sequences in Table 6, and/or the amino acid sequence vp3 capsid protein is selected from the sequences in Table 7.

### Promoter

The invention may contain a muscle specific promoter or another promoter. The promoter may be linked to the nucleic acid sequence so that the transcription preferably occurs within myocytes. Promoter regions enable the host cells to transcribe the transgene only in those cell types and tissues or organs in which the desired protein should be created. Here, the muscle specific promoter is included because it is principally desired that the proteins only be translated in myocytes. Specificity of the cell type into which the nucleic acid is delivered and thus the proteins translated is desired because of the adverse effects that may ensue from delivering the nucleic acid and having it translated in cells in which that nucleic acid and thus protein is not needed.

In some embodiments, the muscle specific promoter yields increased muscle cell potency, muscle cell specificity, reduced immunogenicity, or any combination thereof. As used herein the terms “muscle-specific”, “muscle cell specificity”, “muscle cell potency,” “myocyte specific” and the like, refer to the increased specificity, selectivity, or potency, of the muscle-specific targeting moieties and compositions incorporating said muscle-specific targeting moieties of the present invention for myocytes relative to non-muscle cells. In some embodiments, the cell specificity, or selectivity, or potency, or a combination thereof of a muscle-specific targeting moiety or composition incorporating a muscle-specific targeting moiety described herein is at least 2 to at least 500 times more specific, selective, and/or potent for/in a muscle cell relative to a non-muscle cell.

In some embodiments, the myocyte-selective promoter utilized is MHCK7. MHCK7 is a 771 base pair length promoter that is small enough to be included in an AAV vector. MHCK7 directs expression in fast and slow skeletal and cardiac muscle, with low expression in the liver, lung, and spleen. The MHCK7 promoter is associated with high levels of expression in skeletal muscles, including the diaphragm, and includes an enhancer to especially drive expression in the heart and skeletal muscle, whereas expression in off-target tissues is minimal. For example, the promoter may be an MHCK7 promoter with the nucleic acid sequence of SEQ ID NO: 2.

In some embodiments, the promoters described herein are inserted into an AAV protein (e.g., an AAV capsid protein) that has reduced specificity (or no detectable, measurable, or clinically relevant interaction) for one or more non-muscle cell types. Exemplary non-muscle cell types include, but are not limited to, liver, kidney, lung, spleen, central or peripheral nervous system cells, bone, immune, stomach, intestine, eye, skin cells and the like. In some embodiments, the non-muscle cells are liver cells.

The term “operably linked” refers to the association of two or more nucleic acid molecules on a single nucleic acid fragment so that the function of one is affected by the other.

Further exemplary tissue specific promoters include U6 promoter sequence, MHCK7 promoter sequence, CK6 promoter sequence, tMCK promoter sequence, CK5 promoter sequence, MCK promoter sequence, HAS promoter sequence, MPZ promoter sequence, desmin promoter sequence, APOA2 promoter sequence, hAAT promoter sequence, INS promoter sequence, IRS2 promoter sequence, MYH6 promoter sequence, MYL2 promoter sequence, TNNI3 promoter sequence, SYN1 promoter sequence, GFAP promoter sequence, NES promoter sequence, MBP promoter sequence, or TH promoter sequence.

Muscle specific promoters are described in International Patent Application Publications WO 2020/006458 and WO 2021/126880, the contents of each of which are incorporated by reference herein.

Further muscle specific promoters are described in U.S. Pat. Nos. 9,133,482; 10,105,453; 10,301,367; U.S. Patent Publication No. 2020-0360534; PCT International Patent Publication Nos. WO 2020/006458; WO 2021/035120; WO 2021/053124; and WO 2021/077000, the contents of each of which are incorporated by reference herein.

RNA polymerase II promoters that are inducible and/or tissue-specific have been previously described. RNA polymerase promoters are known in the art and further described in U.S. Patent Publication 11,149,288, the contents of which is incorporated by reference herein.

### Alternatively-Spliced Exons

Aspects of the invention comprise alternatively-spliced exons that may be used in the context of viral vectors to effectively regulate the expression of a coding region of the MTM1 gene. In certain embodiments, the alternatively-spliced exons regulate a coding region of interest in a condition-sensitive manner. A condition-sensitive manner means that the alternatively-spliced exon regulates the expression of a coding region of interest in a manner that is controlled or influenced by one or more conditions, including, but not limited to, environmental conditions, intracellular conditions, extracellular conditions, type of cell (e.g., liver cells versus muscle cells), gene expression pattern, or disease state. Accordingly, aspects of the invention comprise regulating expression of the coding region of the MTM1 gene in a condition-sensitive manner, by coupling the expression of a coding region of interest with an alternatively-spliced exon cassette. Alternatively spliced exons are described in PCT International Application No. PCT/US2022/017015, the entirety of the contents of which are incorporated by reference herein.

In some embodiments, the alternatively-spliced exon cassette comprises 1, 2, 3, or 4 alternatively-spliced exons. In some other embodiments, the alternatively-spliced exon cassette comprises 1, 2, 3, 4, 5, 6, 7, 8, 9, 10, 11, 12, 13, 14, 15, 16, 17, 18, 19, 20, 21, 22, 23, 24, or 25 alternatively-spliced exons. In some embodiments, wherein the alternatively-spliced exon cassette comprises more than one alternatively-spliced exon, the alternatively-spliced exons are adjacent. In some embodiments, wherein the alternatively-spliced exon cassette comprises more than one alternatively-spliced exon, the alternatively-spliced exons are not adjacent.

In some embodiments, the alternatively-spliced exon is synthetic or recombinant. In some embodiments, the alternatively-spliced exon is considered to be synthetic or recombinant because it undergoes one or more nucleic acid modifications, relative to the wild-type alternatively-spliced exon. A nucleic acid modification may be a substitution or deletion of one or more nucleotides that form the nucleic acid sequence of the alternatively-spliced exon.

In some embodiments, an alternative exon comprises an ATG start codon at its 3′ end. As will be understood, in some embodiments a wild-type or naturally occurring alternative exon may comprise an ATG start codon at its 3′ end. In such embodiments, the alternative exon may comprise nucleic acid modifications unrelated to the insertion of a heterologous start codon at the 3′ end of the alternative exon. However, it will be further understood that in some embodiments a wild-type or naturally occurring alternative exon may not comprise an ATG start codon at its 3′ end. In such embodiments, modifications are made to the 3′ end of the alternative exon to introduce a heterologous start codon, such that when the alternative exon is spliced-in or retained in the spliced transcript, the downstream coding sequence is translated as a full-length protein. As will be understood, in some embodiments 1, 2, or 3 nucleic acid substitutions may be necessary in order to introduce the heterologous ATG start codon to the 3′ end of the alternative exon, depending on the sequence which is present at the 3′ end of the wild-type or naturally occurring alternative exon. In such embodiments, the 3′ end of the alternatively-spliced exon comprises 1 nucleotide substitution, relative to the wild-type alternatively-spliced exon, to form the ATG start codon. In such embodiments, the 3′ end of the alternatively-spliced exon comprises 2 nucleotide substitutions, relative to the wild-type alternatively-spliced exon, to form the ATG start codon. In such embodiments, the 3′ end of the alternatively-spliced exon comprises 3 nucleotide substitutions, relative to the wild-type alternatively-spliced exon, to form the ATG start codon.

In some embodiments, the modification comprises the insertion of a heterologous start codon or part of a heterologous start codon at the 3′ end of the alternatively-spliced exon (e.g., 1-3 nucleic acids are added to the 3′ end of the alternatively-spliced exon, rather than substituted, to form an ATG start codon).

In some embodiments, wherein the alternative exon comprises 1, 2, or 3 nucleic acid substitutions at the 3′ end to result in a heterologous ATG start codon (e.g., if the wild-type alternatively-spliced exon does not comprise an ATG start codon at its 3′ end), the strength of the 5′ splice site of the alternative exon may be diminished, relative to the strength of the 5′ splice site strength of the wild-type or naturally occurring alternative exon. In such embodiments, one or more additional modifications made be made to the intronic sequence located immediately downstream of the sequence comprising the 3′ end of the alternative exon. In some embodiments, the first 10 nucleotides of the intronic sequence located immediately downstream of the alternatively-spliced exon comprise 1-5 nucleotide substitutions, relative to the naturally occurring or wild-type intronic sequence located immediately downstream of naturally occurring or wild-type alternative exon. In some embodiments, the first 10 nucleotides of the intronic sequence located immediately downstream of the alternatively-spliced exon comprise 1 nucleotide substitution, relative to the naturally occurring or wild-type intronic sequence located immediately downstream of naturally occurring or wild-type alternative exon. In some embodiments, the first 10 nucleotides of the intronic sequence located immediately downstream of the alternatively-spliced exon comprise 2 nucleotide substitutions, relative to the naturally occurring or wild-type intronic sequence located immediately downstream of naturally occurring or wild-type alternative exon. In some embodiments, the first 10 nucleotides of the intronic sequence located immediately downstream of the alternatively-spliced exon comprise 3 nucleotide substitutions, relative to the naturally occurring or wild-type intronic sequence located immediately downstream of naturally occurring or wild-type alternative exon. In some embodiments, the first 10 nucleotides of the intronic sequence located immediately downstream of the alternatively-spliced exon comprise 4 nucleotide substitutions, relative to the naturally occurring or wild-type intronic sequence located immediately downstream of naturally occurring or wild-type alternative exon. In some embodiments, the first 10 nucleotides of the intronic sequence located immediately downstream of the alternatively-spliced exon comprise 5 nucleotide substitutions, relative to the naturally occurring or wild-type intronic sequence located immediately downstream of naturally occurring or wild-type alternative exon. In some embodiments, the 1-5 nucleotide substitutions restore or partially restore the strength of the 5′ splice site of the alternative exon, relative to the strength of the 5′ splice site of the naturally occurring or wild-type alternative exon.

Additionally or alternatively, in some embodiments the modification comprises disrupting or deleting all native start codons located 5′ to the heterologous start codon. In some embodiments, wherein the alternatively-spliced exon cassette comprises more than one alternatively-spliced exon, all native start codons located 5′ to the heterologous start codon of the 5′-most alternatively-spliced exon are disrupted or deleted. Additionally or alternatively, in some embodiments the modification comprises introducing into the alternatively-spliced exon a heterologous, in-frame stop codon at least 50 nucleotides upstream of the next 5′ splice junction. In some embodiments, the alternatively-spliced exon is a nonsense-mediated decay (NMD) exon. In some embodiments, the NMD exon comprises an in-frame stop codon that is at least 50 nucleotides upstream of the next 5′ splice junction.

In some embodiments, the alternatively-spliced exon is considered to be synthetic when it is situated non-naturally (e.g., is linked to a coding sequence to which it would not be linked in wild-type or naturally-occurring conditions), relative to the wild-type alternatively-spliced exon (e.g., is heterologous). In some embodiments, the alternatively-spliced exon is considered to be synthetic when it (i) undergoes one or more nucleic acid modifications, and (ii) is situated non-naturally, relative to the wild-type alternatively-spliced exon.

In some embodiments, the alternatively-spliced exon is a regulatory exon. In some embodiments, the regulatory exon is an alternatively regulated exon (e.g., an exon known to be subject to alternative splicing mechanisms). It will be appreciated that alternative splicing is a process by which exons or portions of exons or noncoding regions within a pre-mRNA transcript are differentially joined or skipped, resulting in multiple protein isoforms being encoded by a single gene.

### Pharmaceutical Composition

Some embodiments of the invention may include any acceptable form of providing the AAV vector to a subject. For example, the AAV vector may be provided to the subject in the form of a composition or formulation comprising the AAV vector. The expression vector of this invention can be formulated and administered to treat a variety of disease states by any means that produces contact of the active ingredient with the agent's site of action in the body of the subject. The compositions, polynucleotides, polypeptides, particles, cells, vector systems and combinations thereof described herein can be contained in a formulation, such as a pharmaceutical formulation. In some embodiments, the formulations can be used to generate polypeptides and other particles that include one or more muscle-specific targeting moieties described herein. In some embodiments, the formulations can be delivered to a subject in need thereof. In some embodiments, component(s) of the engineered AAV capsid system, engineered cells, engineered AAV capsid particles, and/or combinations thereof described herein can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 μg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 μg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, μL, mL, or L.

In embodiments, were engineered AAV capsid particles are included in the formulation, the formulation can contain 1 to 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles. In some embodiments, the formulation can be 0.1 to 100 mL in volume and can contain 1 to 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles.

### Pharmaceutically Acceptable Carriers and Auxiliary Ingredients and Agents

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT (as sold by Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, mannitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g. intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

### EXAMPLES

### Example 1: Efficacy of KT-430 in the MTM1 Knockout Mouse (Study KTS1020)

A novel composition, named KT-430, was tested as a therapeutic candidate for the treatment of XLMTM. KT-430 comprises a novel recombinant muscle-tropic capsid, termed MyoAAV3.8., for delivery of a nucleic acid encoding the MTM1 protein. The MyoAAV3.8 capsid delivers a transgene described in SEQ ID NO: 4.

Recently, MTM1 gene replacement using a first-generation MyoAAV capsid was shown to improve survival, body weight gain and motor function when expressing the human MTM1 coding sequence in the Mtm1 KO mouse, demonstrating that the human protein is efficacious in the mouse. See Tabebordbar, 2021, Cell 184:4919-4938, the entirety of the contents of which are incorporated by reference herein. This is consistent with the high degree of homology of MTM1 across eukaryotes, for example the human protein is 92 and 96% identical to the mouse and canine MTM1 proteins, respectively.

Restoration of functional myotubularin via AAV8-mediated gene therapy to express full-length canine MTM1 under the control of the desmin promoter was efficacious in the MTM1 p.N155K dog model. This AAV8-based vector improved survival and produced durable improvements in contractile muscle force, motor and respiratory function, and histological abnormalities when administered IV at high doses ≥2 E14 vg/kg. These preclinical studies using an AAV8-based gene therapy and desmin promoter predicted the clinical efficacy of AT132 (NCT03199469; ASPIRO), a related AAV8-based gene therapy (AAV8-desmin-hMTM1), thus validating the predictivity of these disease models and their pharmacological relevance.

By the present invention, KT-430, by virtue of its design and particularly its novel muscle-tropic capsid, MyoAAV3.8, was tested for its ability to achieve efficacious levels of transgene expression in skeletal muscle at a dose an order of magnitude lower than the lowest dose of AT132 used in ASPIRO. This dose would substantially reduce the exposure of non-target tissues to KT-430, potentially enabling clinical efficacy (improvements in muscle strength and reduced need for mechanical ventilation) with improved safety and in particular, reduced risk of hepatotoxicity in this population with great unmet medical need.

### Study Objective

The objective of the study was to evaluate the efficacy and biodistribution of KT-430 (MyoAAV3.8-MHCK7-hMTM1) in male MTM/KO mice across a dose-range (3 E11, 1 E12, and 3 E12 vg/kg) 10-weeks after a single IV injection.

### Methods

The Mtm1 KO mouse (B6; 129S-Mtm1^{tm1(Gt(OST290577)Lex}) was obtained and bred by Taconic Biosciences (Model #TF0892). The mouse model does not express MTM1 and exhibits reduced survival, muscle pathology and motor defects consistent with a previously published Mtm1 KO strain. Mice (n=6 hemizygous males/group) were administered a single IV (retro-orbital) injection (10 mL/kg) of vehicle (PBS+35 mM NaCl+0.001% Pluronic F68) or increasing doses of KT-430 at 4 weeks of age. A group of WT male littermates were treated in parallel with vehicle to permit comparisons relative to healthy mice. Mice were dosed at 4 weeks of age, since previous studies have established that AAV-based gene replacement is efficacious when administered shortly following weaning, but before the mice become moribund due to disease progression.

An interim efficacy assessment was conducted 4-weeks post-dose (8-weeks of age) at a time when most of the vehicle treated KO mice are still viable and able to perform functional tests, which included grip strength (Grip Strength Meter, Columbus Instruments), open field activity (Panlab Harvard Apparatus Open-Field Arena/LE800SC) and spontaneous running wheel activity (Med Associates Low Profile Wireless Running Wheel). Shortly thereafter the majority of the vehicle treated mice require humane euthanasia due to disease progression. At the end of the study, 10-weeks post-dose, another functional assessment was performed to assess efficacy of surviving mice by comparison to WT littermates. Mice were euthanized and tibialis, bicep and quadricep were weighed in pairs to assess muscle growth. Select muscle tissues were also evaluated microscopically for reversal of pathological abnormalities, including quantitation of central nuclei and fiber diameter, and staining for nicotinamide adenine dinucleotide (NADH) to assess organelle mislocalization. Heart and liver were also evaluated by light microscopy for potential toxicity. Tissues were assessed for biodistribution, including vector copy number (VCN), hMTM1 transgene mRNA and protein expression.

TABLE 5
Study Design for KTS1020

| | Test | | Formulated | Dose | No. |
|---|---|---|---|---|---|
| Group | Article | Dose | Concentration | Volume | Animals |
| 1 | Vehicle | — | — | 10 | 6 males (wild-type) |
| 2 | Vehicle | — | — | mL/kg | 6 males (knockout) |
| 3 | KT-430 | 3E11 vg/kg | 3E10 vg/mL | | 6 males (knockout) |
| 4 | KT-430 | 1E12 vg/kg | 1E11 vg/mL | | 6 males (knockout) |
| 5 | KT-430 | 3E12 vg/kg | 3E11 vg/mL | | 6 males (knockout) |

Biodistribution of vector genomes was evaluated by digital droplet PCR (ddPCR) using Taqman primers/probes directed towards the 3′ end of the hMTM1 coding sequence. The number of vector genomes was normalized to the number of diploid genomes using the murine telomerase (Tert) reference gene. To measure transgene mRNA, TaqMan assays were developed using the same primers directed towards the 3′ end of the hMTM1 coding sequence. Copy numbers of mRNA were quantitated relative to a standard curve and normalized to the levels of murine Gapdh mRNA as a reference gene. Additionally, the levels of the hMTM1 transgene mRNA were compared to endogenous levels of mouse Mtm1 determined from vehicle treated WT littermates in Group 1. Protein levels of Mtm1 were determined by western blot using an anti-Mtm1 antibody (Abnova).

### Results

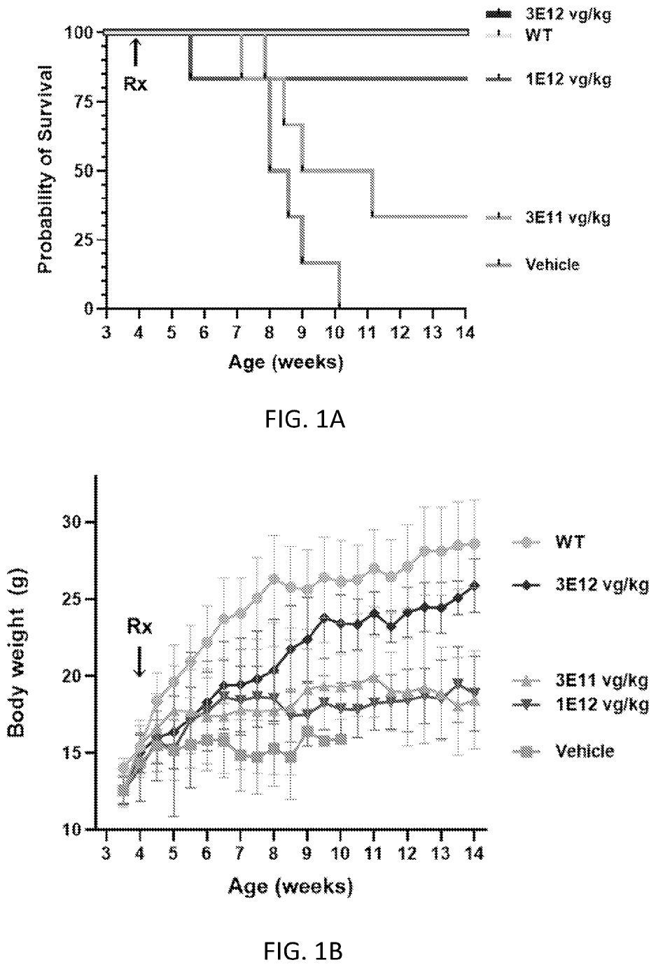

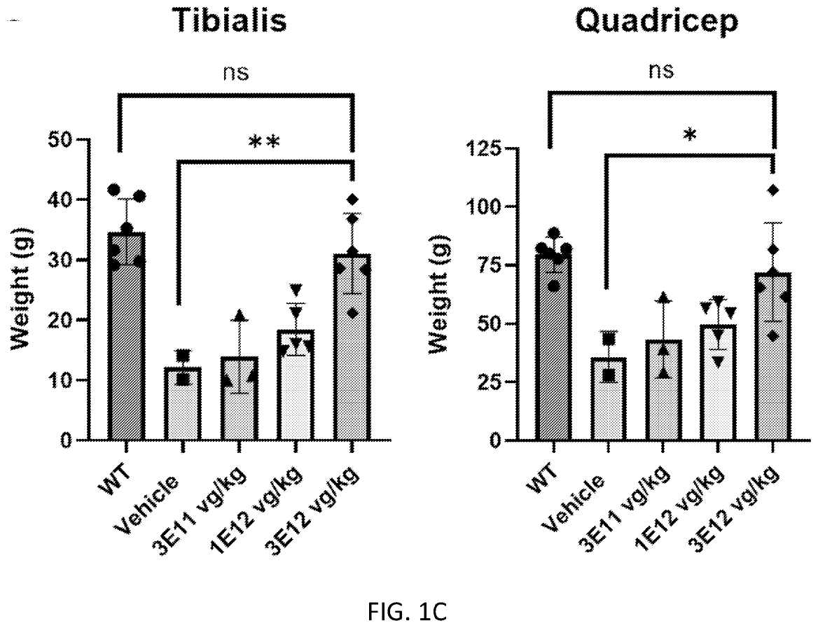

FIG. 1A-C show results from KT-430 administration on survival and growth in Mtm1 KO Mice. Mtm1 KO mice were evaluated for survival, body weight and terminal muscle weight. FIG. 1A, Survival; FIG. 1B, Body weight, and FIG. 1C muscle weight of tibilias anterior and quadricep. Asterisks indicate statistical difference from vehicle treated KO mice (*p<0.05; **p<0.01).

Treatment with KT-430 led to a dose-dependent increase in survival. All vehicle treated KO mice had to be euthanized moribund between 8 and 10 weeks of age due to disease progression, consistent with the reduced survival reported elsewhere in Mtm1 KO mice. By contrast, all 6 KO mice in the high dose group (3 E12 vg/kg) survived to scheduled necropsy at week 10. In the low (3 E11 vg/kg) and mid (1 E12 vg/kg) dose group, 2/6 and 5/6 animals survived to week 10, respectively. There was also a dose-dependent increase in muscle weight (quadricep, tibialis) and body weight in KT-430 treated KO mice. Muscle and body weight in the high dose treated KO mice were not significantly different from WT animals at necropsy at week 10.

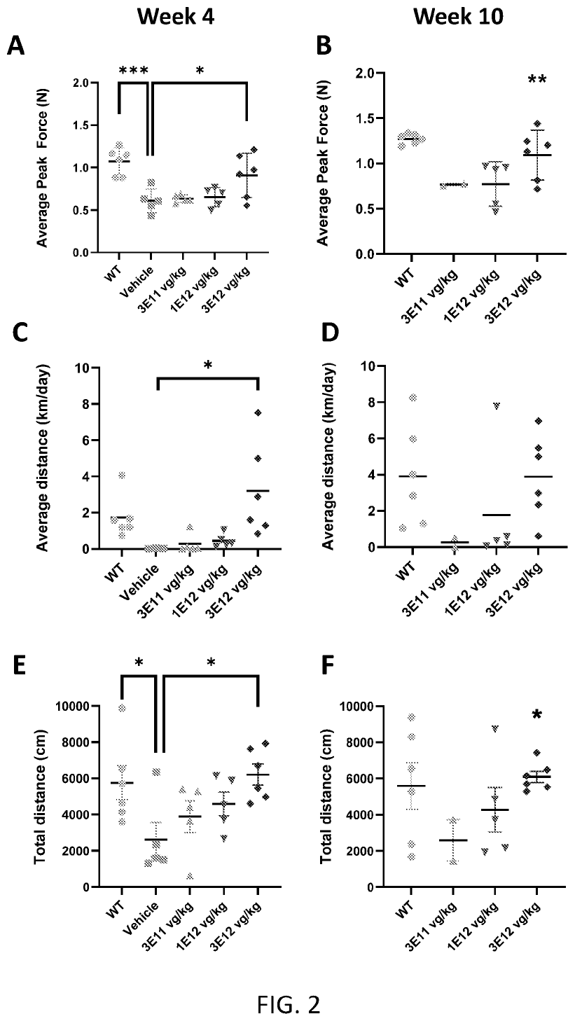

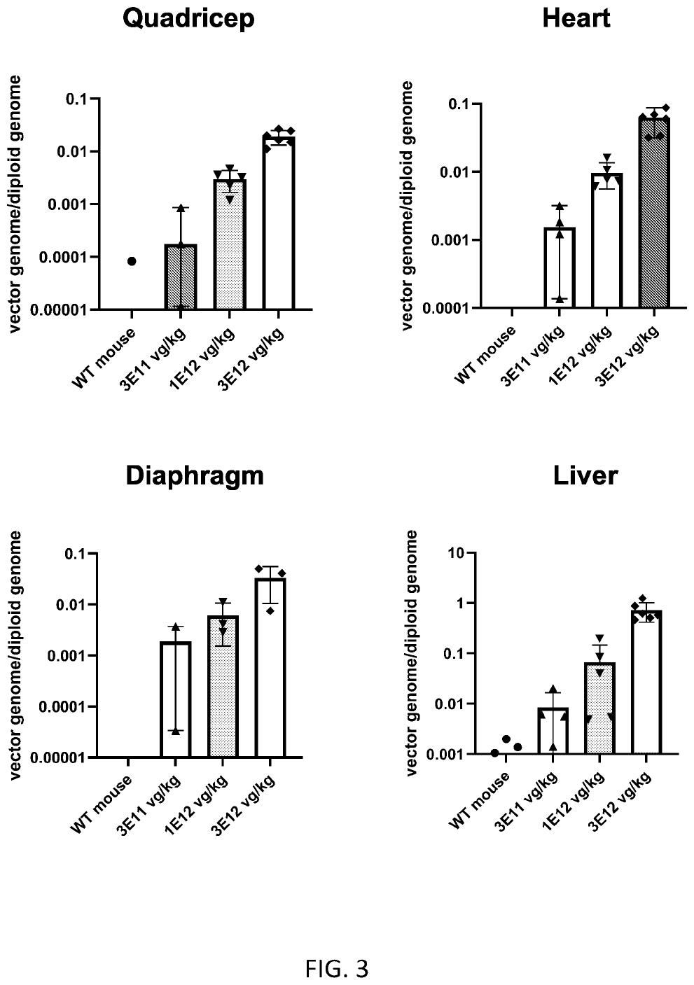

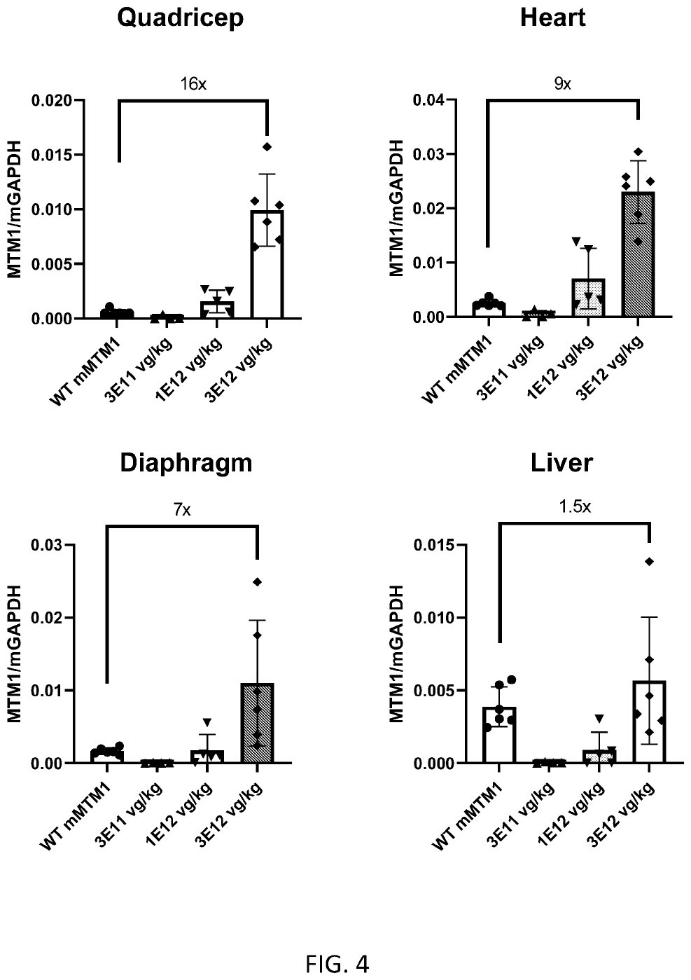

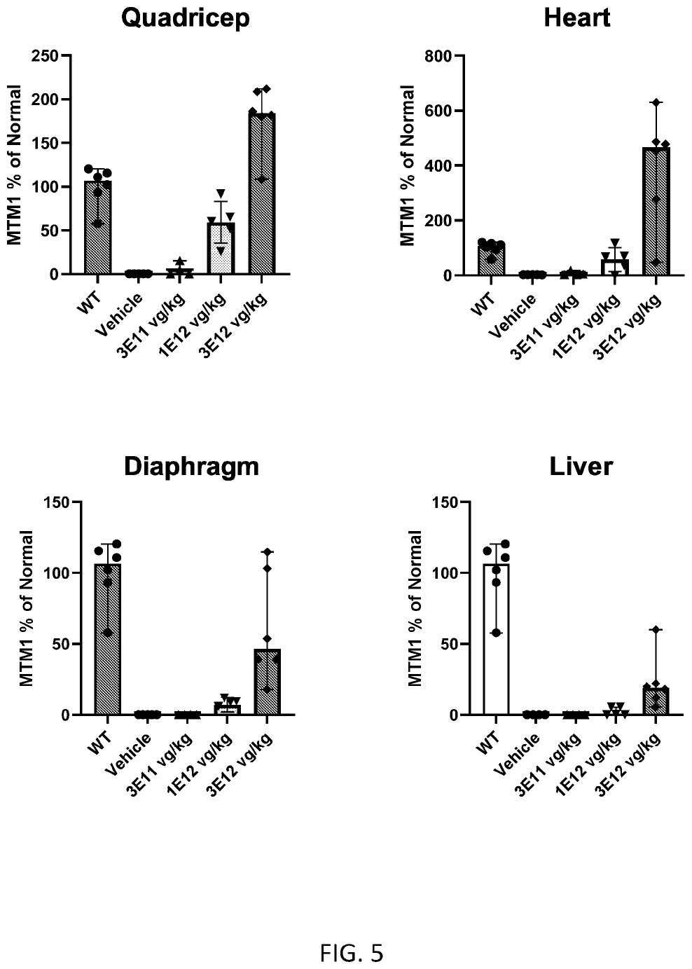

FIG. 2 shows results from KT-430 administration on muscle function in Mtm1 KO Mice. Mtm1 KO mice were treated at 4 weeks of age and evaluated for motor function at week 4 post-dose when vehicle treated KO mice are still viable. The mice were then evaluated a second time prior to necropsy at week 10 post-dose. A-B) Average peak force (newtons) of grip strength based on 5 repeat determinations per mouse. C-D) Spontaneous running wheel activity (average distance per day in kilometers) generated over 7-day (week 4) or 8-day period (week 10). E-F) Open field activity (total distance in centimeters) measured over 30 min period in open field arena. Asterisks indicate statistical difference from vehicle treated KO mice (*p<0.05; **p<0.01; ***p<0.001).

Vehicle treated Mtm1 KO mice exhibit a dramatic reduction in grip strength, open field activity (distance traveled) and spontaneous running wheel activity (average daily distance run over 7- to 8-day period) compared to WT littermates when measured at 8 weeks of age. Treatment with KT-430 resulted in a dose-dependent improvement in these measures of motor function, however due to high inter-animal variability the results only achieved statistical significance at the high dose.

Vehicle treated Mtm1 KO mice exhibit the expected pathological features of XLMTM, including reduced myofiber size, internal/central nucleation and abnormal localization of organelles in quadricep and bicep. Mtm1 KO mice treated with KT-430 showed a dose-dependent reversal of these histological features in quadricep and bicep at doses of 1 E12 and 3 E12 vg/kg, but no clear improvement at the low dose of 3 E11 vg/kg. This is consistent with the minimal improvements in motor function at this dose level. Liver and heart were histologically normal all doses tested of KT-430, further supporting the safety of KT-430. There were also no relevant changes in serum chemistry, including alanine transaminase, aspartate transaminase, alkaline phosphatase, bilirubin or creatine kinase in KT-430 treated mice.

### Vector Copy Number (VCN)

Biodistribution of vector genome DNA was evaluated in a subset of tissues in surviving KT-430-treated mice at week 10.

FIG. 3 shows the biodistribution of KT-430 in Mtm1 KO Mice. Abbreviations used in FIG. 3 include: KO=knockout; Mtm1=mouse myotubularin gene; Tert=telomerase; WT=wild type. Vector copy numbers per diploid genome in mice dosed with increasing doses of KT-430 (mean±Standard Deviation). Vector copy numbers are normalization to the mouse Tert gene (n=3-6/group).

Treatment with KT-430 results in a dose-dependent increase in vector genomes per diploid genome in all tissues evaluated. Levels in the liver at the efficacious dose of 3 E12 vg/kg was approximately 1 vg/dg. Levels in muscle and heart were less than 0.1 vg/dg.

### Transgene Expression

FIG. 4 shows dose-dependent expression of hMTM1 mRNA following treatment of KT-430 in Mtm1 KO Mice. Transgene mRNA levels (mean±Standard Deviation) of the hMTM1 transgene from mice treated with increasing doses of KT-430 (n=3-5/group) relative to endogenous murine Mtm1 mRNA was measured in vehicle treated WT mice. All data are normalized to the levels of murine Gapdh.

KT-430 treatment resulted in a dose-dependent increase in hMTM1 transgene mRNA in muscle and heart at the mid and high dose of 1 E12 and 3 E12 vg/kg. By comparison to endogenous murine Mtm1 mRNA levels, a dose of 1 E12 vg/kg results in hMTM1 mRNA levels that were approximately similar to physiological levels in muscle and heart. The high dose of 3 E12 vg/kg resulted in supra-physiological levels of transgene mRNA in muscle and heart (7 to 16-fold normal). The low dose of 3 E11 vg/kg produced detectable, but very low levels of transgene mRNA that were sub-physiological. Interestingly in liver the high dose of 3 E12 vg/kg produced physiological levels of transgene mRNA indicating that the MHCK7 promoter has weak activity in the liver.

FIG. 5 shows the dose-dependent expression of MTM1 Protein Following Treatment of KT-430 in Mtm1 KO Mice. hMTM1 protein levels (mean±Standard Deviation) from mice treated with increasing doses of KT-430 (n=3-5/group) relative to endogenous murine MTM1 in WT animals.

As expected, based on mRNA levels, KT-430 produced a dose-dependent in increase in hMTM1 protein expression in muscle, heart and liver. There was no appreciable MTM1 expression at 3 E11 vg/kg in any tissue, however sub-physiological MTM1 expression (<100% of normal) was observed at 1 E12 vg/kg in muscle and heart, and supra-physiological expression (>100% of normal) at 3 E12 vg/kg.

### CONCLUSION

In summary, these results demonstrate that KT-430 treatment results in a dose-dependent improvement in survival, body weight growth, and motor function. KT-430 is fully efficacious at a dose of 3 E12 vg/kg, consistent with achieving at least 100% of normal MTM1 expression in muscle. The mid dose of 1 E12 vg/kg showed some evidence of efficacy based on survival and trend towards improvements in body weight, muscle weight and motor function. This is consistent with the lower sub-physiological expression of hMTM1 mRNA and protein. Based on the efficacy and safety data, and consistent MTM1 protein expression in muscle at 10 weeks post-dose, treatment of KT-430 does not appear to be associated with an anti-transgene immune response or hepatotoxicity. These results support the biological activity of KT-430 and demonstrate that MTM1 gene replacement can reverse disease symptoms when expressed at physiological levels. The results also support the safety of KT-430.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### Additional Sequences

We claim:

1. An adeno-associated virus (AAV) vector comprising:
a capsid protein comprising at least one modification that results in preferential targeting of the AAV vector to muscle tissue; and
a nucleic acid encoding a full-length MTM1 protein.

2. The AAV vector of claim 1, wherein the capsid protein comprises at least one modification that is an insertion between any two contiguous amino acids between amino acids 262-269, 327-332, 382-386, 452-460, 488-505, 527-539, 545-558, 581-593, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

3. The AAV vector of claim 1, wherein the capsid protein comprises at least one modification that is a replacement of amino acids 586-88 and an insertion between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

4. The AAV vector of claim 1, wherein the capsid protein comprises at least one modification that is a replacement of amino acids 586-588 and an insertion between amino acids 588 and 589 in an AAV9 capsid polypeptide and wherein the insertion is selected from the sequences in Table 1-4.

5. The AAV vector of claim 1, wherein the vector comprises an AAV vp1 capsid protein, a vp2, capsid protein, and a vp3 capsid protein, wherein;
the amino acid sequence of the vp1 capsid protein is selected from the sequences in Table 5;
the amino acid sequence of the vp2 capsid protein is selected from the sequences in Table 6; and/or
the amino acid sequence of the vp3 capsid protein is selected from the sequences in Table 7.

6. The method of claim 1, wherein the capsid protein further comprises at least one modification that results in reduced liver-tropism of the AAV vector.

7. The AAV vector of claim 1, wherein the nucleic acid encoding the full-length MTM1 protein is operably linked to a muscle specific promoter.

8. The AAV vector of claim 6, wherein the muscle specific promoter is an MHCK7 promoter.

9. The AAV vector of claim 8, wherein the nucleic acid encoding a full-length MTM1 protein comprises an alternatively-spliced exon cassette downstream of the muscle specific promoter, wherein the alternatively-spliced exon cassette comprises an ATG start codon at the 3′ end of the cassette.

10. The AAV vector of claim 10, wherein the alternatively-spliced exon cassette comprises a skeletal muscle-specific exon.

11. The AAV vector of claim 11, wherein the alternatively spliced exon cassette promotes skeletal muscle expression of the nucleic acid.

12. A method of treating X-linked myotubular myopathy (XLMTM), the method comprising administering to a subject afflicted with XLMTM a composition comprising:
an adeno-associated virus (AAV) vector comprising:
a capsid protein comprising at least one modification that results in preferential targeting of the AAV vector to muscle tissue; and
a nucleic acid encoding a full-length MTM1 protein.

13. The method claim 12, wherein the capsid protein comprises at least one modification that is an insertion between any two contiguous amino acids between amino acids 262-269, 327-332, 382-386, 452-460, 488-505, 527-539, 545-558, 581-593, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

14. The method of claim 12, wherein the capsid protein comprises at least one modification that is a replacement of amino acids 586-88 and an insertion between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

15. The method of claim 12, wherein the capsid protein comprises at least one modification that is a replacement of amino acids 586-588 and an insertion between amino acids 588 and 589 in an AAV9 capsid polypeptide and wherein the insertion is selected from the sequences in Table 1-4.

16. The method of claim 12, wherein the vector comprises an AAV vp1 capsid protein, a vp2, capsid protein, and a vp3 capsid protein, wherein;
the amino acid sequence of the vp1 capsid protein is selected from the sequences in Table 5;
the amino acid sequence of the vp2 capsid protein is selected from the sequences in Table 6; and/or
the amino acid sequence of the vp3 capsid protein is selected from the sequences in Table 7.

17. The method of claim 1, wherein the capsid protein further comprises at least one modification that results in reduced liver-tropism of the AAV vector.

18. The method of claim 1, wherein the nucleic acid encoding the full-length MTM1 protein is operably linked to a muscle specific promoter.

19. The method of claim 8, wherein the muscle specific promoter is an MHCK7 promoter.

20. The method of claim 9, wherein the nucleic acid encoding a full-length MTM1 protein comprises an alternatively-spliced exon cassette downstream of the muscle specific promoter, wherein the alternatively-spliced exon cassette comprises an ATG start codon at the 3′ end of the cassette.

21. The method of claim 20, wherein the alternatively-spliced exon cassette comprises a skeletal muscle-specific exon.

22. The method of claim 21, wherein the alternatively spliced exon cassette promotes skeletal muscle expression of the nucleic acid.
